(19) United States
(12) Patent Application Publication
(10) Pub. No.: US 2022/0079931 A1
(43) Pub. Date: Mar. 17, 2022

(54) ESTROGEN RECEPTOR PROTEIN DEGRADERS

(71) Applicant: THE REGENTS OF THE UNIVERSITY OF MICHIGAN, Ann Arbor, MI (US)

(72) Inventors: Shaomeng Wang, Superior Township, MI (US); Jiantao Hu, Ann Arbor, MI (US); Biao Hu, Ann Arbor, MI (US); Mingliang Wang, Ann Arbor, MI (US); Fuming Xu, Ann Arbor, MI (US); Bukeyan Miao, Ann Arbor, MI (US)

(21) Appl. No.: 17/420,417

(22) PCT Filed: Dec. 19, 2019
(86) PCT No.: PCT/US19/67311

§ 371 Date: Jul. 2, 2021

Related U.S. Application Data
(60) Provisional application No. 62/787,996, filed on Jan. 3, 2019.

(51) Int. Cl. A61K 31/454; A61K 31/381; A61K 47/54; A61K 31/427; A61K 31/40; A61P 35/00; A61K 31/337; A61K 31/566; A61K 31/7068; A61K 31/704; A61K 31/513; A61K 31/475; A61K 31/138; A61K 31/519; A61K 31/565

(52) U.S. Cl. CPC A61K 31/454; A61K 31/565; A61K 47/54; A61K 47/545; A61K 31/427; A61K 31/40; A61P 35/00; A61K 31/337; A61K 31/566; A61K 31/7068; A61K 31/704; A61K 31/513; A61K 31/475; A61K 31/138; A61K 31/519; A61K 31/381

(57) ABSTRACT

The present disclosure provides compounds represented by Formula (I): A-L-B and the salts or solvates thereof, wherein A, L, and B are as defined in the specification. Compounds having Formula I are estrogen receptor degraders useful for the treatment of cancer.

### BACKGROUND OF THE INVENTION

### Field of the Invention

The present disclosure provides heterobifunctional small molecules as estrogen receptor (ER) protein degraders. ER degraders useful for the treatment of a variety of diseases including breast cancer.

### Background

Breast cancer (BC) is one of the most common malignancies in women, worldwide. Based on the status of the tumor receptor, breast cancer can be further subdivided into estrogen receptor-positive (ER+), human epidermal growth factor receptor 2 (HER2)-positive (HER2+) and triple-negative subtypes.¹ ER+ breast cancer occurs in approximately 80% of newly diagnosed breast cancer cases.² As members of the nuclear receptor family, estrogen receptors ERα and ERβ are transcription factors regulating gene expression and mediating the biological effects of the estrogens. Both ERα and ERβ are widely expressed in different tissues and ERα is considered to be the major medium which transduces the estrogen signaling in the female reproductive tract and mammary glands.³ ERα has therefore been pursued as a promising therapeutic target in multiple pathological settings, particularly in cancer and osteoporosis, and this is highlighted by the clinical success of tamoxifen for the treatment of ER+BC and raloxifene for the prevention and treatment of osteoporosis in postmenopausal women.^{4, 5}

Although inhibition of estrogen synthesis by aromatase inhibitors and inhibition of ER pathway signaling by selective estrogen receptor modulators (SERM) (FIG. 1) have demonstrated considerable clinical benefit in the treatment of ER+BC, the development of intrinsic and acquired resistance to those chug classes presents an impediment for patients with advanced and metastatic breast cancer.^{6, 7} While there are clearly multiple resistance mechanisms to aromatase inhibitors and SERMs, recent studies have demonstrated that in the most of the cases of resistance, continued dependence on ERα signaling for tumor growth and disease progression is retained and the ER protein remains a principal driver in ER+ metastatic breast cancer.^{8, 9}

Selective estrogen receptor degraders (SERD) are small molecules that target ERα for proteasome-dependent degradation. Currently, fulvestrant (5, FIG. 1) is the only SERD that has been approved for the treatment of postmenopausal women with advanced ER+ breast cancer with standard endocrine therapies.^{10, 11} The clinical success enjoyed by fulvestrant suggests that degradation of the ER protein is beneficial to patients with ER+ breast cancer, particularly those whose disease continues to progress after standard endocrine therapies. Because fulvestrant has poor solubility and is not orally bioavailable, it is administered clinically by means of a monthly intramuscular injection.^{12, 13} To address the shortcomings of fulvestrant, orally bioavailable SERD molecules have been developed and a number of them are currently being evaluated in clinical trials as new therapies for the treatment of ER+, metastatic BC.¹⁴⁻¹⁹

The proposed mechanism of action for traditional SERDs such as fulvestrant is induction of misfolding of the ER protein, which ultimately leads to proteasome-dependent ERα protein degradation.²⁰ The SERD molecules are typically potent and effective in inducing degradation of ER protein in ER+ breast cancer cells, but they are only able to achieve partial degradation of the ER protein.^{21, 22} Consequently, novel therapeutic agents, which can achieve more complete degradation of ER, could be more efficacious than the traditional SERD molecules for the treatment of ER+ metastatic breast cancer.

The proteolysis targeting chimera (PROTAC) concept was first introduced in 2001,²³ with the objective of induction of selective target protein degradation by hijacking the cellular E3 ubiquitination ligase systems.²⁴⁻²⁸ PROTACs are heterobifunctional small-molecules containing a ligand, which binds to the target protein of interest, and another ligand for an E3 ligase system. These two ligands are tethered together by a chemical linker. The PROTAC strategy has recently gained momentum due in part to the availability of potent and druglike small-molecule ligands for a number of E3 ligase systems, and it has been employed for the design of small-molecule degraders for a number of proteins.²⁹⁻⁴³ Recently, Naito et al. reported several PROTAC-like ERα degraders, which were named Specific and Nongenetic IAP-dependent Protein Erasers (SNIPERs).^{44, 45} They designed ERα SNIPER molecules using an ERα antagonist and a ligand for inhibitors of apoptosis protein (IAPs), which are E3 ligases. However, while SNIPER ER degraders effectively induce partial degradation of the ER protein, they also induce auto-ubiquitylation and proteasomal degradation of the E3 ligase, the cIAP1 protein, potentially limiting their therapeutic efficacy.

There is a need in the art for additional ER degraders to treat breast cancer and other diseases.

### BRIEF SUMMARY OF THE INVENTION

In one aspect, the present disclosure provides heterobifunctional small molecules represented by any one or more of Formulae I-V, below, and the pharmaceutically acceptable salts and solvates, e.g., hydrates, thereof, collectively referred to herein as “Compounds of the Disclosure.” Compounds of the Disclosure are estrogen receptor degraders and are thus useful in treating diseases or conditions wherein degradation of the estrogen receptor provides a therapeutic benefit to a patient.

In another aspect, the present disclosure provides methods of treating a condition or disease by administering a therapeutically effective amount of a Compound of the Disclosure to a patient, e.g., a human, in need thereof. The disease or condition is treatable by degradation of the estrogen receptor, for example, a cancer, e.g., breast cancer.

In another aspect, the present disclosure provides a method of degrading of the estrogen receptor in an individual, comprising administering to the individual an effective amount of at least one Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating diseases or conditions wherein degradation of the estrogen receptor provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in treatment of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In another aspect, the present disclosure provides methods of preparing Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims. It is to be understood that both the foregoing summary and the following detailed description are exemplary and explanatory only, and are not restrictive of the invention as claimed.

### BRIEF DESCRIPTION OF DRAWINGS

FIG. 1 is an image showing the Western blotting analysis of ER protein in MCF-7 cells treated with Compounds of the Disclosure and control compounds. Cells were treated with different compounds for 4 h and whole cell lysates were then analyzed by Western blotting to examine the level of ER protein. GADPH protein was used for the loading control. The numbers below the panel represent the ERα/GADPH ratio normalized with the DMSO control at 100.

FIG. 2 is an image showing the Western blotting analysis of ER protein in the MCF-7 cells treated with indicated compounds at 1 nM, 10 nM and 100 nM. MCF-7 cells were treated with different compounds for 4 h and whole cell lysates were analyzed by Western blotting to examine the level of ER protein. GADPH protein was used for the loading control. The numbers below the panel represent the ERα/GADPH ratio normalized with the DMSO control at 100.

FIG. 3 is an image showing the Western blotting analysis of ER protein in the MCF-7 cells treated with indicated compounds at 1 nM, 10 nM and 100 nM. MCF-7 cells were treated with different compounds for 4 h and whole cell lysates were analyzed by Western blotting to examine the level of ER protein. GADPH protein was used for the loading control. The numbers below the panel represent the ERα/GADPH ratio normalized with the DMSO control at 100.

FIG. 4 is an image showing the Western blotting analysis of ER protein in the MCF-7 cells treated with indicated compounds at 1 nM, 10 nM and 100 nM. MCF-7 cells were treated with different compounds for 4 h and whole cell lysates were analyzed by Western blotting to examine the level of ER protein. GADPH protein was used for the loading control. The numbers below the panel represent the ERα/GADPH ratio normalized with the DMSO control at 100.

FIG. 5 is an image showing the Western blotting analysis of ER protein in the MCF-7 cells treated with indicated compounds at 1 nM, 10 nM and 100 nM. MCF-7 cells were treated with different compounds for 4 h and whole cell lysates were analyzed by Western blotting to examine the level of ER protein. GADPH protein was used for the loading control. The numbers below the panel represent the ERα/GADPH ratio normalized with the DMSO control at 100.

FIG. 6 is an image showing the Western blotting analysis of ER protein in the MCF-7 cells treated with indicated compounds at 1 nM, 10 nM and 100 nM. MCF-7 cells were treated with different compounds for 4 h and whole cell lysates were analyzed by Western blotting to examine the level of ER protein. GADPH protein was used for the loading control. The numbers below the panel represent the ERα/GADPH ratio normalized with the DMSO control at 100.

FIG. 7 is an image showing the ERα degradation dose-response Western blotting for compound 32 at 4 h in MCF-7 cells.

FIG. 8 is an image showing the ERα degradation dose-response Western blotting for compound 32 at 4 h in T47D cells.

FIG. 9 is an image showing the time course of ERα degradation by Western blotting by compound 32 (30 nM) and fulvestrant (30 nM) in the MCF-7 cells.

FIG. 10 is an image showing the time course of ERα degradation by Western blotting by compound 32 (30 nM) and fulvestrant (30 nM) in the T47D cells.

FIG. 11 is an image showing that ERα degradation is dependent on VHL, ER and proteasome by Western blotting analysis. MCF-7 cells were pretreated with VHL ligand 11 (1 μM), or ER ligand raloxifene (1) (1 μm), or the proteasome inhibitor carfilzomib (1 μM) for 2 h, followed by treatment with DMSO or compound 32 (30 nM) for 4 h. Then whole-cell lysates were analyzed by Western blotting.

FIG. 12 is an image showing that ERα degradation is dependent on VHL, ER and proteasome by Western blotting analysis. MCF-7 cells were pretreated with VHL ligand 11 (+, 0.5 μM; ++, 1 μM; +++, 5 μM; ++++, 10 μM) for 2 h, followed by treatment with DMSO or compound 32 (30 nM) for 4 h. Then whole-cell lysates were analyzed by Western blotting.

### DETAILED DESCRIPTION OF THE INVENTION

### I. Compounds of the Disclosure

Compounds of the Disclosure are heterobifunctional ER receptor degraders. In one embodiment, Compounds of the Disclosure are compounds represented by Formula I:

A-L-B I,

wherein:

A is a radical of an estrogen receptor modulator selected from the group consisting of:

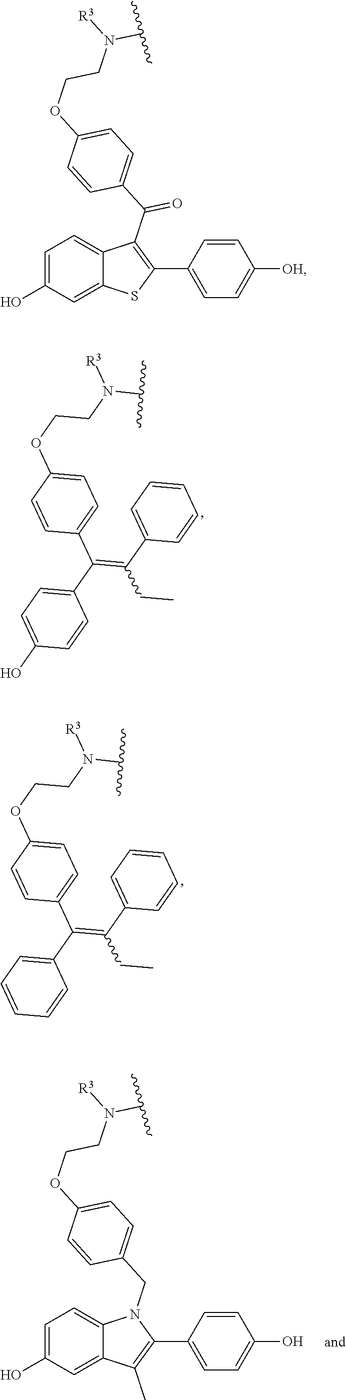

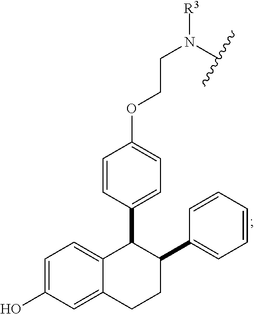

R³ is selected from the group consisting of C₁-C₆ alkyl, C₃-C₈ cycloalkyl, and (C₃-C₈ cycloalkyl)C₁-C₄ alkyl;

L is a linker; and

B is a radical of an E3 ligase ligand selected from the group consisting of:

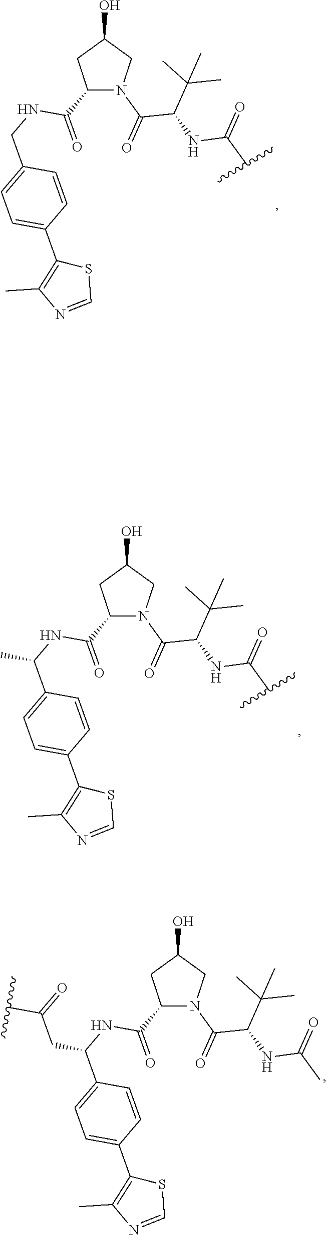

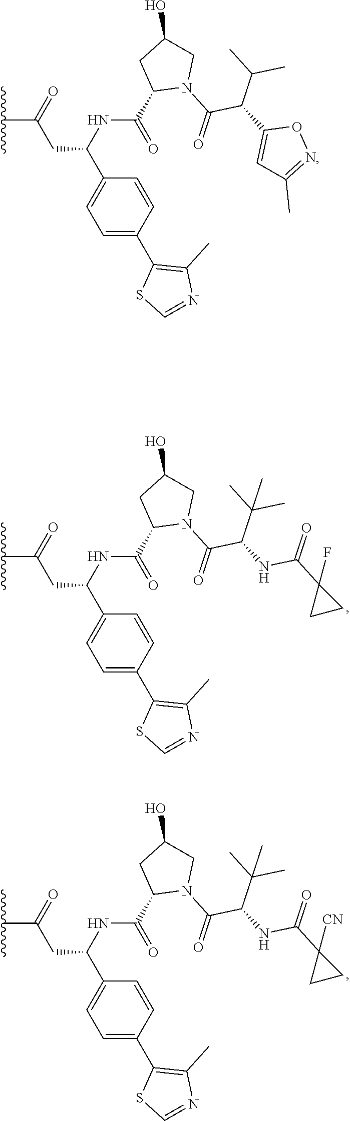

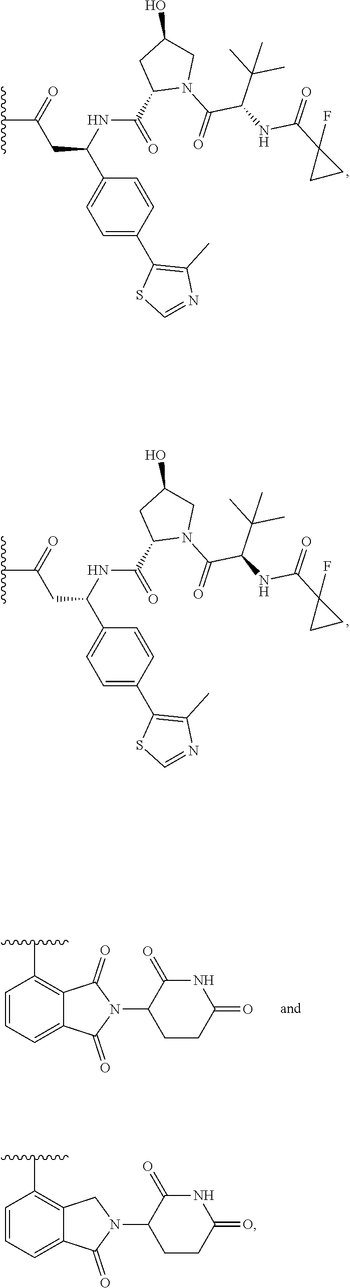

or a pharmaceutically acceptable salt or solvate thereof.

In another embodiment, Compounds of the Disclosure are compounds represented by Formula I, wherein A is selected from the group consisting of:

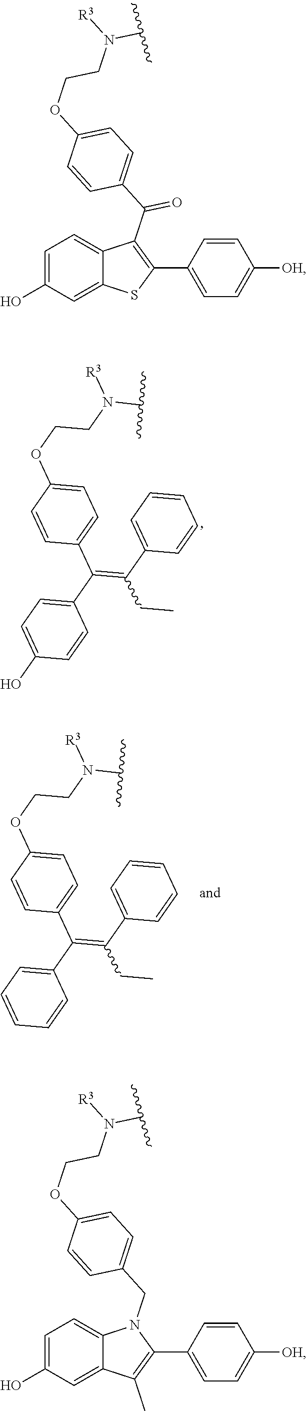

or a pharmaceutically acceptable salt or solvate thereof.

In another embodiment, Compounds of the Disclosure are compounds represented by Formula I, wherein B is selected from the group consisting of:

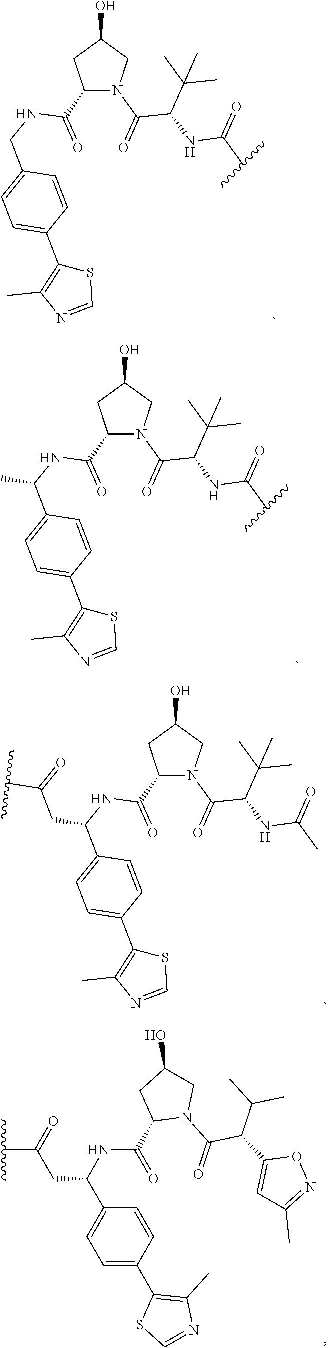

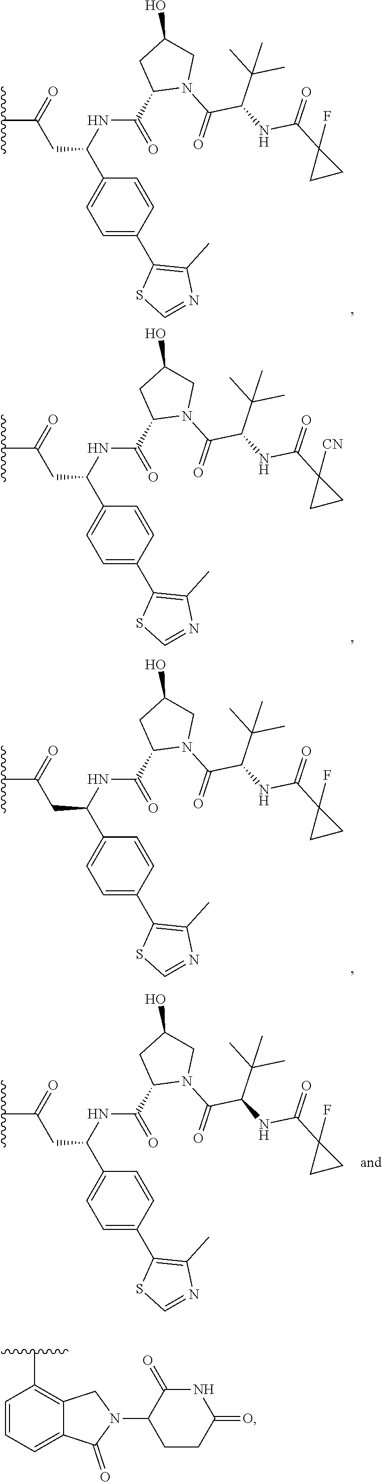

or a pharmaceutically acceptable salt or solvate thereof.

In another embodiment, Compounds of the Disclosure are compounds represented by Formula II:

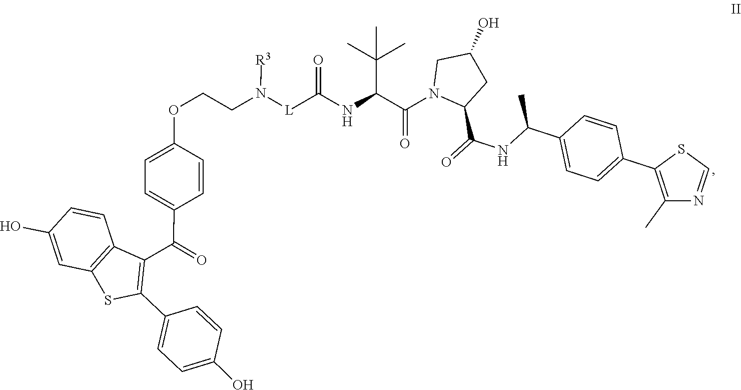

wherein R³ and L are as defined in connection with Formula I, or a pharmaceutically acceptable salt or solvate thereof.

In another embodiment, Compounds of the Disclosure are compounds represented by Formula III:

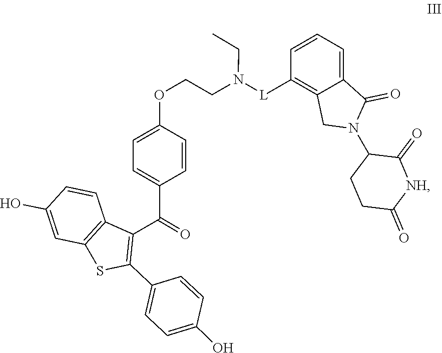

wherein L is as defined in connection with Formula I, or a pharmaceutically acceptable salt or solvate thereof.

In another embodiment, Compounds of the Disclosure are compounds represented by any one of Formulae I-III, wherein:

L is —X-L¹-Z—;

X is selected from the group consisting of —C≡C—, —O—, —C(═O)N(R^{1a})—, and —N(R^{3a})—; or

X is absent;

Z is selected from the group consisting of —C≡C—, —O—, —C(═O)N(R^{2a})—, and —N(R^{4a})—; or

Z is absent;

L¹ is selected from the group consisting of alkylenyl, heteroalkylenyl, and —W¹—(CH₂)ₘ—W²—(CH₂)ₙ—

W¹ is absent; or

W¹ is selected from the group consisting of phenylenyl, heteroarylenyl, heterocyclenyl, and cycloalkylenyl;

W² is selected from the group consisting of phenylenyl, heteroarylenyl, heterocyclenyl, and cycloalkylenyl;

m is 0, 1, 2, 3, 4, 5, 6, or 7;

n is 0, 1, 2, 3, 4, 5, 6, 7, or 8; and

R^{1a} is selected from the group consisting of hydrogen and C₁₋₄ alkyl;

R^{2a} is selected from the group consisting of hydrogen and C₁₋₄ alkyl;

R^{3a} is selected from the group consisting of hydrogen and C₁₋₄ alkyl; and

R^{4a} is selected from the group consisting of hydrogen and C₁₋₄ alkyl, or a pharmaceutically acceptable salt or solvate thereof.

In another embodiment, Compounds of the Disclosure are compounds represented by any one of Formulae I-III, wherein L is selected from the group consisting of:

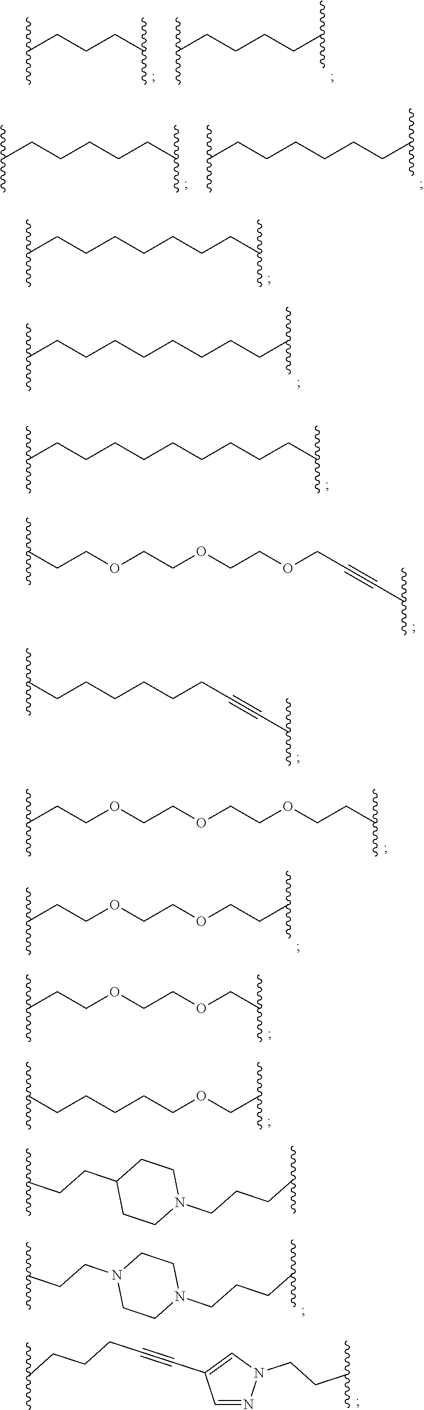

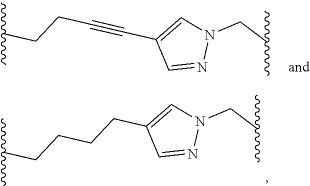

or a pharmaceutically acceptable salt or solvate thereof.

In another embodiment, Compounds of the Disclosure are compounds represented by Formula IV:

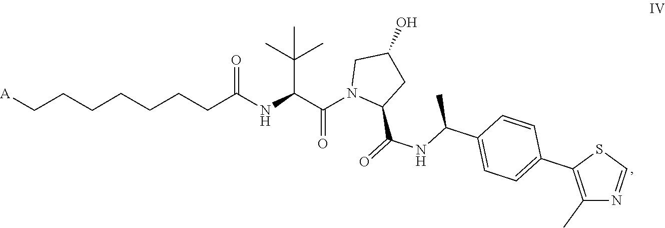

wherein A is as defined in connection with Formula I, or a pharmaceutically acceptable salt or solvate thereof.

In another embodiment, Compounds of the Disclosure are compounds represented by Formula V:

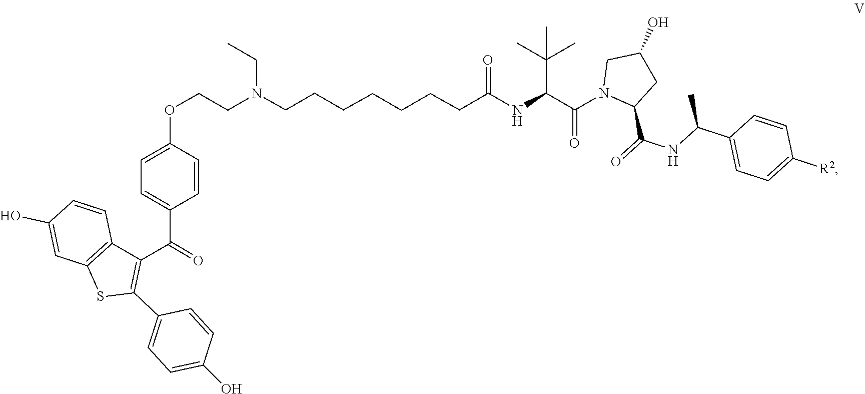

wherein:

R¹ is selected from the group consisting of hydrogen and C₁-C₃ alkyl; and

R² is selected from the group halo, cyano, C₂-C₄ alkynyl, C₁-C₆ alkyl, and C₃-C₆ cycloalkyl or a pharmaceutically acceptable salt or solvate thereof.

In another embodiment, Compounds of the Disclosure are compounds having Formulae I-III, and the salts or solvates thereof, wherein X is —C≡C—.

In another embodiment, Compounds of the Disclosure are compounds having Formulae I-III, and the salts or solvates thereof, wherein X is —N(H)—.

In another embodiment, Compounds of the Disclosure are compounds having Formulae I-III, and the salts or solvates thereof, wherein W¹ is

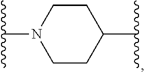 [SMILES: *C1CCN(*)CC1]

and the carbon atom of

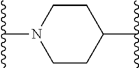 [SMILES: *C1CCN(*)CC1]

is attached to L¹.

In another embodiment, Compounds of the Disclosure are compounds having Formulae I-III, and the salts or solvates thereof, wherein L is C₁₋₁₂ alkylenyl.

In another embodiment, Compounds of the Disclosure are compounds having Formulae I-III, and the salts or solvates thereof, wherein L is selected from the group consisting of —CH₂—, —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, and —CH₂(CH₂)₆CH₂—.

In another embodiment, Compounds of the Disclosure are compounds having Formula I, and the salts or solvates thereof, wherein L is 3- to 12-membered heteroalkylenyl.

In another embodiment, Compounds of the Disclosure are compounds having Formulae I-III, and the salts or solvates thereof, wherein L is -A-(CH₂)ₘ—W—(CH₂)ₙ— and A is absent.

In another embodiment, Compounds of the Disclosure are compounds having Formulae I-III, and the salts or solvates thereof, wherein L is selected from the group consisting of:

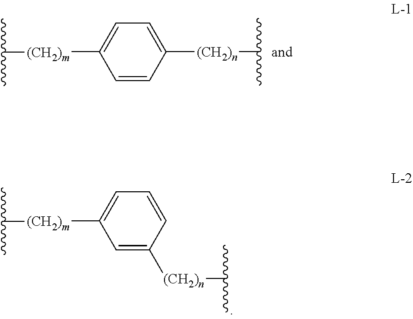

In another embodiment, Compounds of the Disclosure are compounds having Formulae I-III, and the salts or solvates thereof, wherein L is -A-(CH₂)ₘ—W—(CH₂)ₙ—, A is absent, and W is 5-membered heteroarylenyl. In another embodiment, m is 0.

In another embodiment, Compounds of the Disclosure are compounds having Formulae I-III, and the salts or solvates thereof, wherein:

L is selected from the group consisting of:

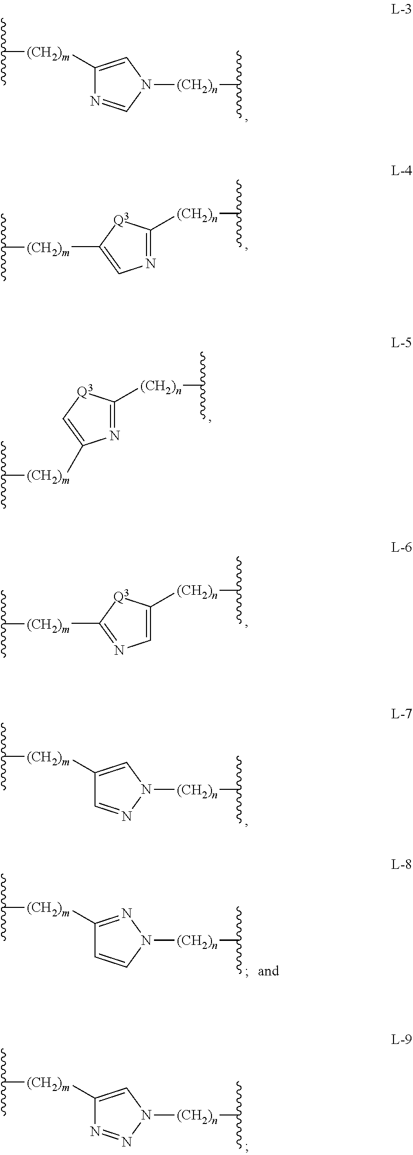

Q³ is selected from the group consisting of —O—, —S—, and —N(R⁶)—; and

R⁶ is selected from the group consisting of hydrogen and C₁₋₄ alkyl.

In another embodiment, Compounds of the Disclosure are compounds having Formulae I-III, and the salts or solvates thereof, wherein L is -A-(CH₂)ₘ—W—(CH₂)ₙ—, A is absent, and W is 6-membered heteroarylenyl. In another embodiment, m is 0.

In another embodiment, Compounds of the Disclosure are compounds having Formulae I-III, and the salts or solvates thereof, wherein L is selected from the group consisting of:

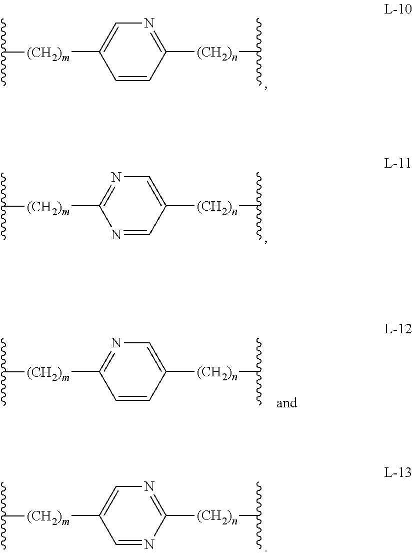

In another embodiment, Compounds of the Disclosure are compounds having Formulae I-III, and the salts or solvates thereof, wherein L is -A-(CH₂)ₘ—W—(CH₂)ₙ—, A is absent, and W is heterocyclenyl. In another embodiment, m is 0.

In another embodiment, Compounds of the Disclosure are compounds having Formulae I-III, and the salts or solvates thereof, wherein L is selected from the group consisting of:

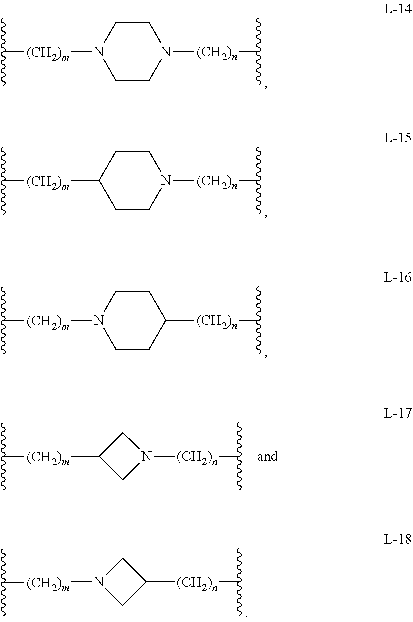

In another embodiment, Compounds of the Disclosure are compounds having Formulae I-III, and the salts or solvates thereof, wherein L is selected from the group consisting of:

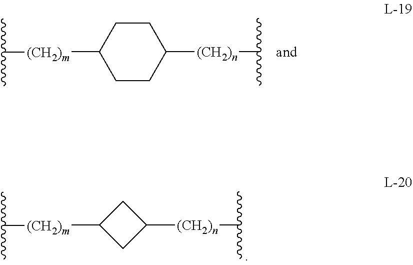

In another embodiment, Compounds of the Disclosure are compounds having Formulae I-III, and the salts or solvates thereof, wherein L is selected from the group consisting of:

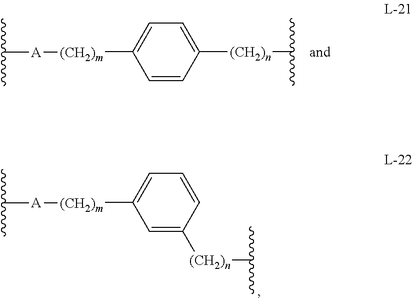

In another embodiment, Compounds of the Disclosure are compounds having Formulae I-III, and the salts or solvates thereof, wherein L is selected from the group consisting of:

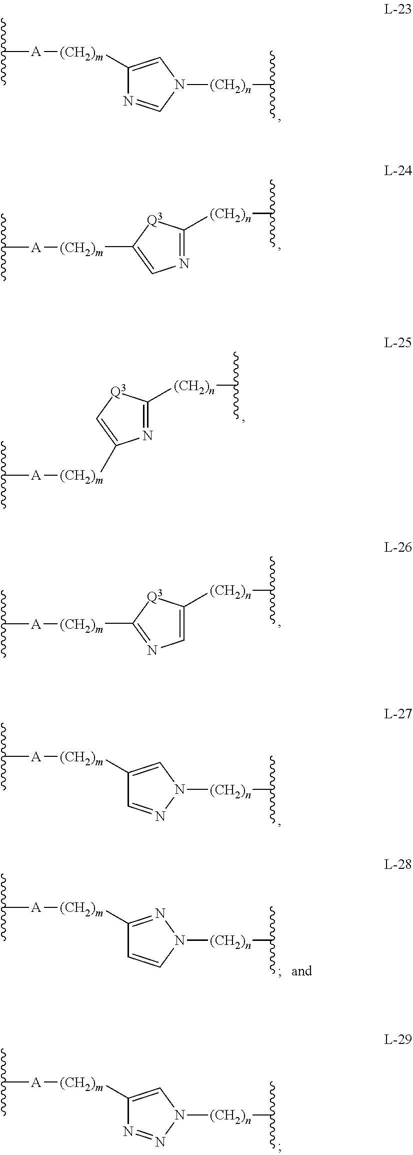

Q³ is selected from the group consisting of —O—, —S—, and —N(R⁶)—; and

R⁶ is selected from the group consisting of hydrogen and C₁₋₄ alkyl.

In another embodiment, Compounds of the Disclosure are compounds having Formulae I-III, and the salts or solvates thereof, wherein L is selected from the group consisting of:

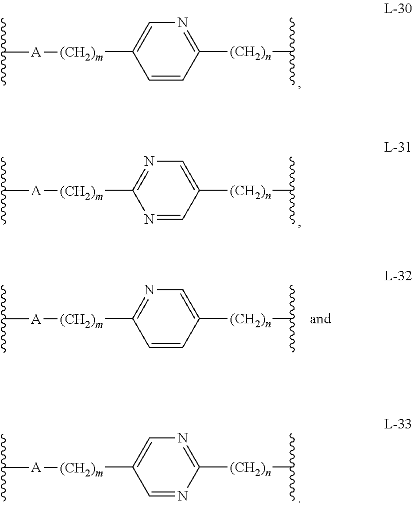

In another embodiment, Compounds of the Disclosure are compounds having Formulae I-III, and the salts or solvates thereof, wherein L is selected from the group consisting of:

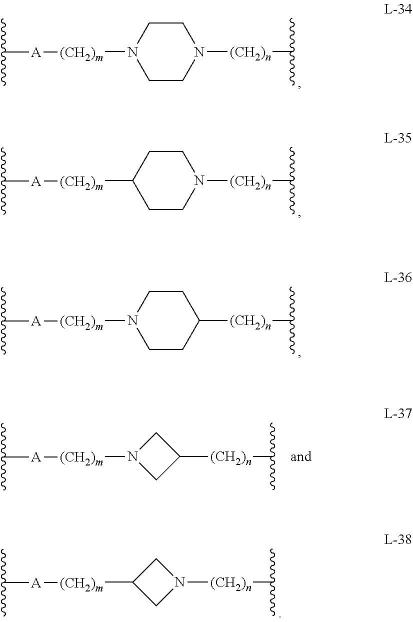

In another embodiment, Compounds of the Disclosure are compounds having Formulae I-III, and the salts or solvates thereof, wherein L is selected from the group consisting of:

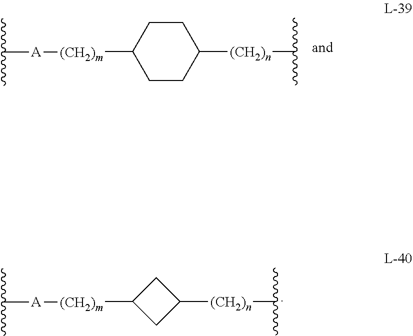

Salts, hydrates, and solvates of the Compounds of the Disclosure can also be used in the methods disclosed herein. The present disclosure further includes all possible stereoisomers and geometric isomers of Compounds of the Disclosure to include both racemic compounds and optically active isomers. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure and the heterobifunctional target protein degraders prepared from Compounds of the Disclosure, including pharmaceutically acceptable salts. As used herein, the pharmaceutical “pharmaceutically acceptable salt” refers to salts or zwitterionic forms of Compounds of the Disclosure and the heterobifunctional target protein degraders prepared from Compounds of the Disclosure. Salts of Compounds of the Disclosure and the heterobifunctional target protein degraders prepared from Compounds of the Disclosure can be prepared during the final isolation and purification of the compounds or separately by reacting the compound with an acid having a suitable cation. The pharmaceutically acceptable salts of Compounds of the Disclosure and the heterobifunctional target protein degraders prepared from Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include compounds of Compounds of the Disclosure as well as pharmaceutically acceptable salts, hydrates, or solvates thereof.

The present disclosure encompasses the preparation and use of solvates of Compounds of the Disclosure and the heterobifunctional target protein degraders prepared from Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure and the heterobifunctional target protein degraders prepared from Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Therapeutic Methods of the Disclosure

Compounds of the Disclosure degrade ER protein and are useful in the treatment of a variety of diseases and conditions. In particular, Compounds of the Disclosure are useful in methods of treating a disease or condition wherein degradation ER proteins provides a benefit, for example, cancers and proliferative diseases. The therapeutic methods of the disclosure comprise administering a therapeutically effective amount of a Compound of the Disclosure to an individual in need thereof. The present methods also encompass administering a second therapeutic agent to the individual in addition to the Compound of the Disclosure. The second therapeutic agent is selected from drugs known as useful in treating the disease or condition afflicting the individual in need thereof, e.g., a chemotherapeutic agent and/or radiation known as useful in treating a particular cancer.

The present disclosure provides Compounds of the Disclosure as ER protein degraders for the treatment of a variety of diseases and conditions wherein degradation of ER proteins has a beneficial effect. Compounds of the Disclosure typically have a binding affinity (IC₅₀) to ER of less than 100 μM, e.g., less than 50 μM, less than 25 μM, and less than 5 μM, less than about 1 μM, less than about 0.5 μM, or less than about 0.1 μM. In one embodiment, the present disclosure relates to a method of treating an individual suffering from a disease or condition wherein degradation of ER proteins provides a benefit comprising administering a therapeutically effective amount of a Compound of the Disclosure to an individual in need thereof.

Since Compounds of the Disclosure are degraders of ER protein, a number of diseases and conditions mediated by ER can be treated by employing these compounds. The present disclosure is thus directed generally to a method for treating a condition or disorder responsive to degradation of ER in an animal, e.g., a human, suffering from, or at risk of suffering from, the condition or disorder, the method comprising administering to the animal an effective amount of one or more Compounds of the Disclosure.

The present disclosure is further directed to a method of degrading ER protein in an animal in need thereof, said method comprising administering to the animal an effective amount of at least one Compound of the Disclosure.

The methods of the present disclosure can be accomplished by administering a Compound of the Disclosure as the neat compound or as a pharmaceutical composition. Administration of a pharmaceutical composition, or neat compound of a Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered. Further provided are kits comprising a Compound of the Disclosure and, optionally, a second therapeutic agent useful in the treatment of diseases and conditions wherein degradation of ER protein provides a benefit, packaged separately or together, and an insert having instructions for using these active agents.

In one embodiment, a Compound of the Disclosure is administered in conjunction with a second therapeutic agent useful in the treatment of a disease or condition wherein degradation of ER protein provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered from a single composition or two separate compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more dose of the second therapeutic agent can be administered. The Compound of the Disclosure therefore can be used in conjunction with one or more second therapeutic agents, for example, but not limited to, anticancer agents.

Diseases and conditions treatable by the methods of the present disclosure include, but are not limited to, cancer and other proliferative disorders. In one embodiment, a human patient is treated with a Compound of the Disclosure, or a pharmaceutical composition comprising a Compound of the Disclosure, wherein the compound is administered in an amount sufficient to degrade ER protein in the patient.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by degrading ER protein. In one embodiment, the cancer is breast cancer.

In methods of the present disclosure, a therapeutically effective amount of a Compound of the Disclosure, typically formulated in accordance with pharmaceutical practice, is administered to a human being in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the ER protein degrader that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate. Buffers and pH modifiers can also be added to stabilize the pharmaceutical composition.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

### III. Definitions

The term “estrogen receptor modulator” as used herein refers to a class of drugs that act on the estrogen receptor, including both SERMs and SERDs. Representative estrogen receptor modulators include, but are not limited to:

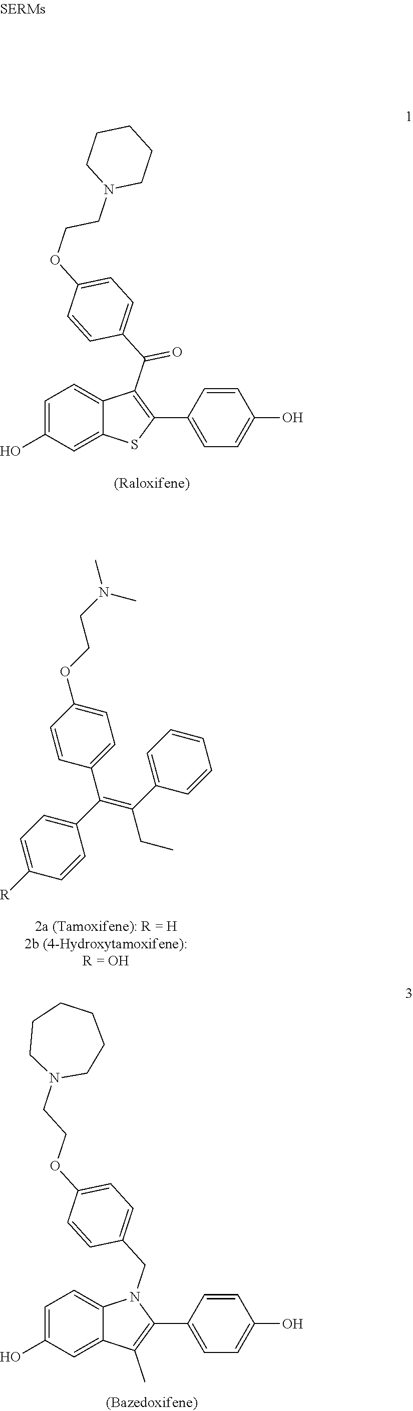

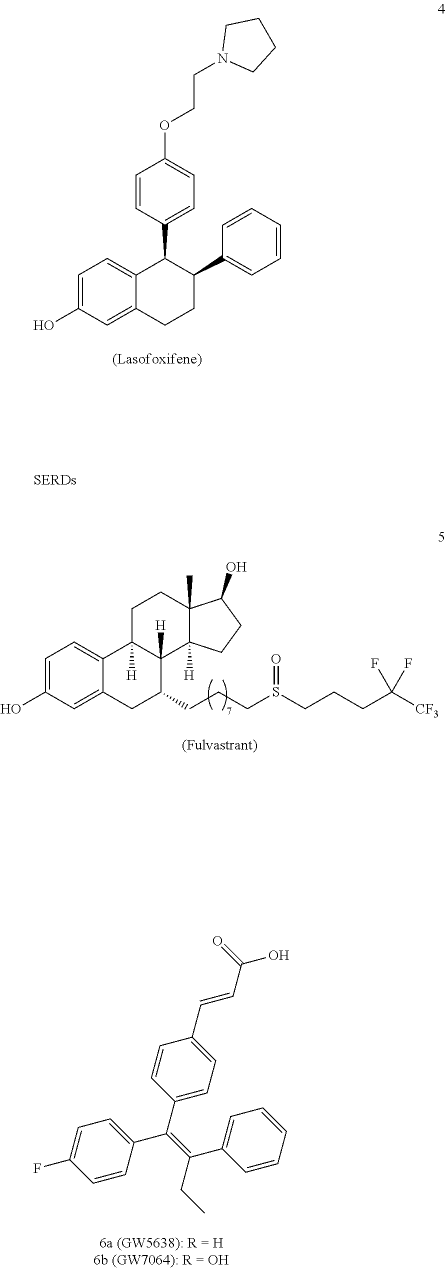

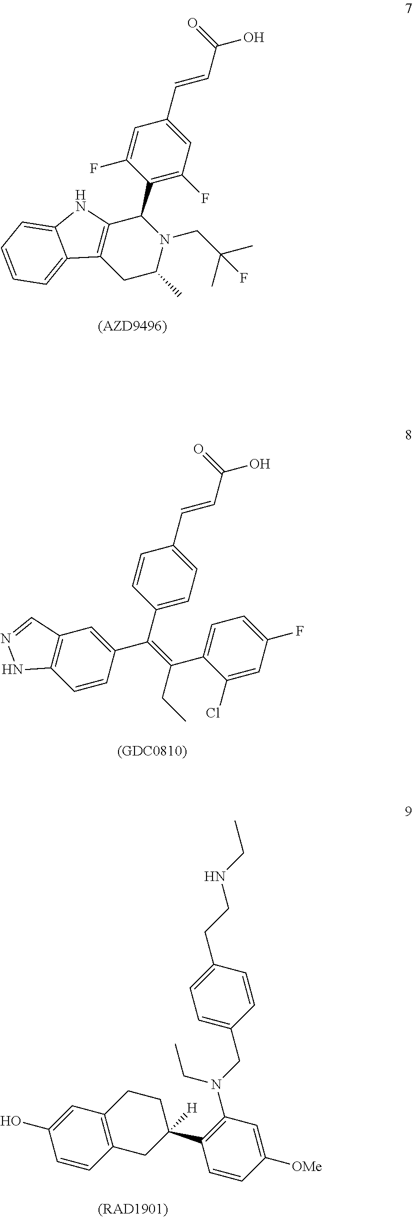 [SMILES: C[C@@H]1Cc2c([nH]c3ccccc23)[C@@H](c2c(F)cc(/C=C/C(=O)O)cc2F)N1CC(C)(C)F ; CC/C(=C(/c1ccc(/C=C/C(=O)O)cc1)c1ccc2[nH]ncc2c1)c1ccc(F)cc1Cl ; CCNCCc1ccc(CN(CC)c2cc(OC)ccc2[C@@H]2CCc3cc(O)ccc3C2)cc1]

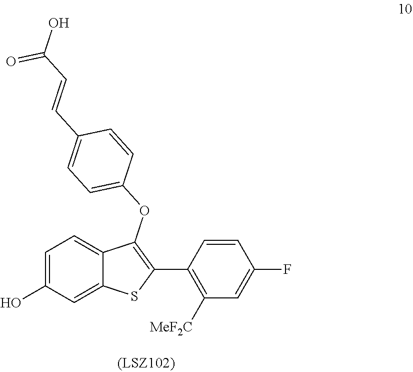

The term “radical of an estrogen receptor modulator” as used herein refers to the chemical species lacking an atom, e.g., hydrogen, or group of atoms, e.g., —CH₃, from a parent estrogen receptor modulator. For example, the absence of —CH₃ from tamoxifene (2a) provides the following radical of an estrogen receptor modulator:

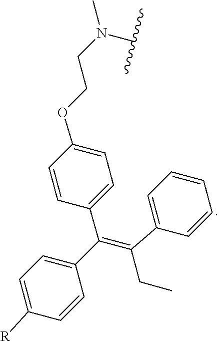

The absence of a hydrogen atom or group of atoms allows for the linkage of the parent estrogen receptor modulator to an E3 ubiquitin ligase protein ligand to give a heterobifunctional compound having Formula I as defined above.

The term “E3 ligase ligand” as herein refers to a compound that binds, e.g., inhibits, an E3 ubiquitin ligase protein, including the von Hippel-Lindau protein (VHL). Ligands for E3 ubiquitin ligase proteins are known to those of ordinary skill in the art. Exemplary non-limiting ligands for an E3 ubiquitin ligase protein include phthalimide-based chugs such as thalidomide or a VHL ligand including, but not limited to, the VHL ligands of Chart 1.

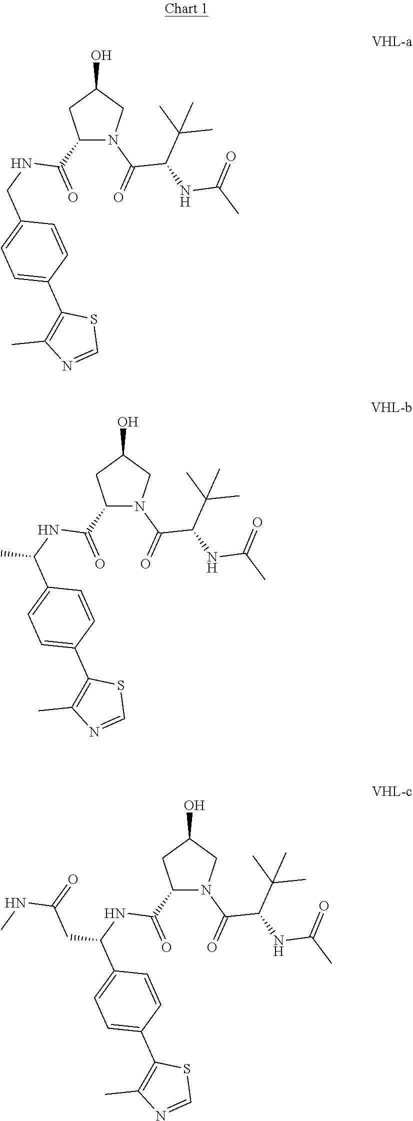

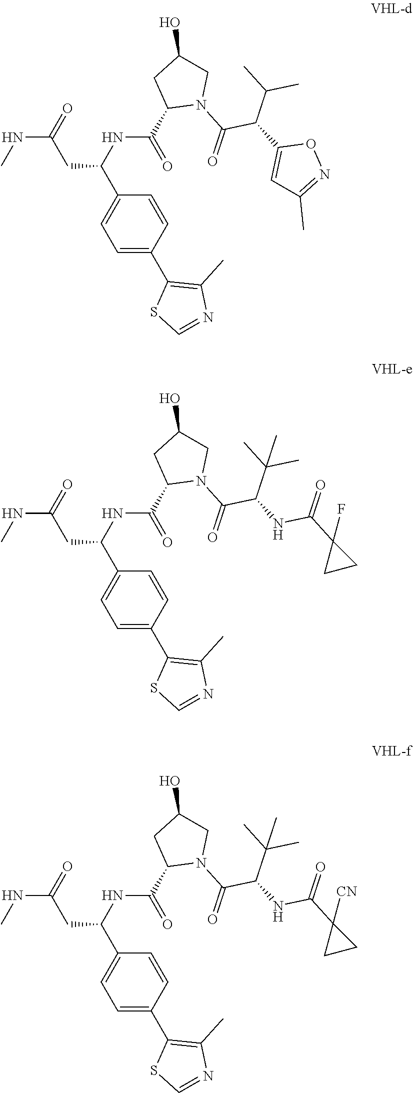 [SMILES: CNC(=O)C[C@H](NC(=O)[C@@H]1C[C@@H](O)CN1C(=O)[C@@H](c1cc(C)no1)C(C)C)c1ccc(-c2scnc2C)cc1 ; CNC(=O)C[C@H](NC(=O)[C@@H]1C[C@@H](O)CN1C(=O)[C@@H](NC(=O)C1(F)CC1)C(C)(C)C)c1ccc(-c2scnc2C)cc1 ; CNC(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1]

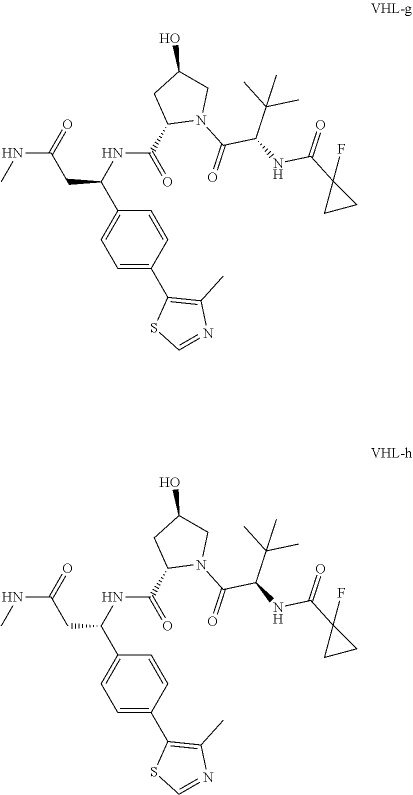 [SMILES: CNC(=O)C[C@@H](NC(=O)[C@@H]1C[C@@H](O)CN1C(=O)[C@@H](NC(=O)C1(F)CC1)C(C)(C)C)c1ccc(-c2scnc2C)cc1 ; CNC(=O)C[C@H](NC(=O)[C@@H]1C[C@@H](O)CN1C(=O)[C@H](NC(=O)C1(F)CC1)C(C)(C)C)c1ccc(-c2scnc2C)cc1]

The phrase “radical of an E3 ligase ligand” refers to chemical species lacking an atom, e.g., hydrogen, or group of atoms, e.g., —CH₃, from a parent E3 ligase ligand. For example, the absence of —CH₃ from VHL-a, see above, provides the following radical of an E3 ligase ligand:

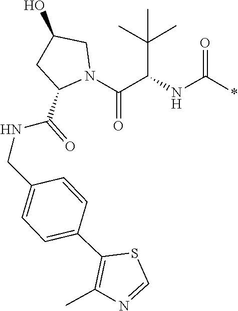

The absence of hydrogen of thalidomide provides the following radical of an E3 ligase ligand:

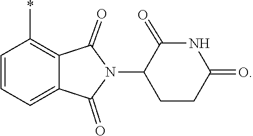

The absence of a hydrogen atom or group of atoms allows for the linkage of the parent E3 ligase ligand to an estrogen receptor modulator to give a heterobifunctional compound having Formula I as defined above

The term “linker” as used herein refers to a divalent chemical moiety capable of tethering a radical of an estrogen receptor antagonist to a radical of an E3 ligase ligand.

The term “about,” as used herein, includes the recited number ±10%. Thus, “about 10” means 9 to 11.

In the present disclosure, the term “halo” as used by itself or as part of another group refers to —Cl, —F, —Br, or —I.

In the present disclosure, the term “nitro” as used by itself or as part of another group refers to —NO₂.

In the present disclosure, the term “cyano” as used by itself or as part of another group refers to —CN.

In the present disclosure, the term “hydroxy” as used by itself or as part of another group refers to —OH.

In the present disclosure, the term “alkyl” as used by itself or as part of another group refers to unsubstituted straight- or branched-chain aliphatic hydrocarbons containing from one to twelve carbon atoms, i.e., C₁₋₂₀ alkyl, or the number of carbon atoms designated, e.g., a C₁ alkyl such as methyl, a C₂ alkyl such as ethyl, a C₃ alkyl such as propyl or isopropyl, a C₁₋₃ alkyl such as methyl, ethyl, propyl, or isopropyl, and so on. In one embodiment, the alkyl is a C₁₋₁₀ alkyl. In another embodiment, the alkyl is a C₁₋₆ alkyl. In another embodiment, the alkyl is a C₁₋₄ alkyl. In another embodiment, the alkyl is a straight chain C₁₋₁₀ alkyl. In another embodiment, the alkyl is a branched chain C₃₋₁₀ alkyl. In another embodiment, the alkyl is a straight chain C₁₋₆ alkyl. In another embodiment, the alkyl is a branched chain C₃₋₆ alkyl. In another embodiment, the alkyl is a straight chain C₁₋₄ alkyl. In another embodiment, the alkyl is a branched chain C₁₋₄ alkyl. In another embodiment, the alkyl is a straight or branched chain C₁₋₄ alkyl. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

In the present disclosure, the term “heteroalkyl” as used by itself or part of another group refers to unsubstituted straight- or branched-chain aliphatic hydrocarbons containing from three to thirty chain atoms, i.e., 3- to 30-membered heteroalkyl, or the number of chain atoms designated, wherein at least one —CH₂— is replaced with at least one —O—, —N(H)—, or —S—. The —O—, N(H)—, or —S— can independently be placed at any interior position of the aliphatic hydrocarbon chain so long as each —O—, N(H)—, or —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. In another embodiment, four —CH₂— groups are replaced with four —O— groups. Non-limiting exemplary heteroalkyl groups include:

—CH₂OCH₃;

—CH₂OCH₂CH₂CH₃;

—CH₂CH₂CH₂OCH₃;

—CH₂OCH₂CH₂OCH₃; and

—CH₂OCH₂CH₂OCH₂CH₂OCH₃.

In the present disclosure, the term “alkylenyl” as used herein by itself or part of another group refers to a divalent form of an alkyl group. In one embodiment, the alkylenyl is a divalent form of a C₁₋₁₂ alkyl. In one embodiment, the alkylenyl is a divalent form of a C₁₋₁₀ alkyl. In one embodiment, the alkylenyl is a divalent form of a C₁₋₈ alkyl. In one embodiment, the alkylenyl is a divalent form of a C₁₋₆ alkyl. In another embodiment, the alkylenyl is a divalent form of a C₁₋₄ alkyl. Non-limiting exemplary alkylenyl groups include:

—CH₂—,

—CH₂CH₂—,

—CH₂CH₂CH₂—,

—CH₂(CH₂)₂CH₂—,

—CH(CH₂)₃CH₂—,

—CH₂(CH₂)₄CH₂—,

—CH₂(CH₂)₅CH₂—,

—CH₂CH(CH₃)CH₂—, and

—CH₂C(CH₃)₂CH₂—.

In the present disclosure, the term “heteroalkylenyl” as used herein by itself or part of another group refers to a divalent form of a heteroalkyl group. In one embodiment, the heteroalkylenyl is a divalent form of a 3- to 12-membered heteroalkyl. In another embodiment, the heteroalkylenyl is a divalent form of a 3- to 10-membered heteroalkyl. In another embodiment, the heteroalkylenyl is a divalent form of a 3- to 8-membered heteroalkyl. In another embodiment, the heteroalkylenyl is a divalent form of a 3- to 6-membered heteroalkyl. In another embodiment, the heteroalkylenyl is a divalent form of a 3- to 4-membered heteroalkyl. In another embodiment, the heteroalkylenyl is a radical of the formula: —(CH₂)ₒO—(CH₂CH₂O)ₚ—(CH₂)_{q}—, wherein o is 2 or 3; p is 0, 1, 2, 3, 4, 5, 6, or 7; and q is 2 or 3. In another embodiment, the heteroalkylenyl is a radical of the formula: —(CH₂)ᵣO—(CH₂)ₛ—O(CH₂)ₜ—, wherein r is 2, 3, or 4; s is 3, 4, or 5; and t is 2 or 3. Non-limiting exemplary heteroalkylenyl groups include:

—CH₂OCH₂—;

—CH₂CH₂OCH₂CH₂—;

—CH₂OCH₂CH₂CH₂—;

—CH₂CH₂OCH₂CH₂CH₂—;

—CH₂CH₂OCH₂CH₂OCH₂CH₂—; and

—CH₂CH₂OCH₂CH₂OCH₂CH₂O—.

In the present disclosure, the term “optionally substituted alkyl” as used by itself or as part of another group means that the alkyl as defined above is either unsubstituted or substituted with one, two, or three substituents independently chosen from nitro, haloalkoxy, aryloxy, aralkyloxy, alkylthio, sulfonamido, alkylcarbonyl, arylcarbonyl, alkylsulfonyl, arylsulfonyl, carboxy, carboxyalkyl, cycloalkyl, and the like. In one embodiment, the optionally substituted alkyl is substituted with two substituents. In another embodiment, the optionally substituted alkyl is substituted with one substituent. Non-limiting exemplary optionally substituted alkyl groups include —CH₂CH₂NO₂, —CH₂SO₂CH₃CH₂CH₂CO₂H, —CH₂CH₂SO₂CH₃, —CH₂CH₂COPh, and —CH₂C₆H₁₁.

In the present disclosure, the term “cycloalkyl” as used by itself or as part of another group refers to saturated and partially unsaturated (containing one or two double bonds) cyclic aliphatic hydrocarbons containing one to three rings having from three to twelve carbon atoms (i.e., C₃₋₁₂ cycloalkyl) or the number of carbons designated. In one embodiment, the cycloalkyl group has two rings. In one embodiment, the cycloalkyl group has one ring. In another embodiment, the cycloalkyl group is chosen from a C₃₋₈ cycloalkyl group. In another embodiment, the cycloalkyl group is chosen from a C₃₋₆ cycloalkyl group. Non-limiting exemplary cycloalkyl groups include cyclopropyl, cyclobutyl, cyclopentyl, cyclohexyl, cycloheptyl, cyclooctyl, norbomyl, decalin, adamantyl, cyclohexenyl, and cyclopentenyl, cyclohexenyl.

In the present disclosure, the term “optionally substituted cycloalkyl” as used by itself or as part of another group means that the cycloalkyl as defined above is either unsubstituted or substituted with one, two, or three substituents independently chosen from halo, nitro, cyano, hydroxy, amino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, and (heterocyclo)alkyl. In one embodiment, the optionally substituted cycloalkyl is substituted with two substituents. In another embodiment, the optionally substituted cycloalkyl is substituted with one substituent.

In the present disclosure, the term “cycloalkylenyl” as used herein by itself or part of another group refers to a divalent form of an optionally substituted cycloalkyl group. Non-limiting examples of a 5 cycloalkylenyl include:

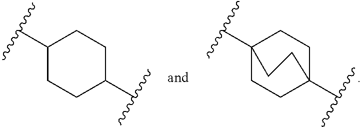 [SMILES: *C1CCC(*)CC1 ; *C12CCC(*)(CC1)CC2]

In the present disclosure, the term “alkenyl” as used by itself or as part of another group refers to an alkyl group as defined above containing one, two or three carbon-to-carbon double bonds. In one embodiment, the alkenyl group is chosen from a C₂₋₆ alkenyl group. In another embodiment, the alkenyl group is chosen from a C₂₋₄ alkenyl group. Non-limiting exemplary alkenyl groups include ethenyl, propenyl, isopropenyl, butenyl, sec-butenyl, pentenyl, and hexenyl.

In the present disclosure, the term “optionally substituted alkenyl” as used herein by itself or as part of another group means the alkenyl as defined above is either unsubstituted or substituted with one, two or three substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, aryl, heteroaryl, or heterocyclo.

In the present disclosure, the term “alkynyl” as used by itself or as part of another group refers to an alkyl group as defined above containing one to three carbon-to-carbon triple bonds. In one embodiment, the alkynyl has one carbon-to-carbon triple bond. In one embodiment, the alkynyl group is chosen from a C₂₋₆ alkynyl group. In another embodiment, the alkynyl group is chosen from a C₂₋₄ alkynyl group. Non-limiting exemplary alkynyl groups include ethynyl, propynyl, butynyl, 2-butynyl, pentynyl, and hexynyl groups.

In the present disclosure, the term “optionally substituted alkynyl” as used herein by itself or as part of another group means the alkynyl as defined above is either unsubstituted or substituted with one, two or three substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, aryl, heteroaryl, or heterocyclo.

In the present disclosure, the term “haloalkyl” as used by itself or as part of another group refers to an alkyl group substituted by one or more fluorine, chlorine, bromine and/or iodine atoms. In one embodiment, the alkyl group is substituted by one, two, or three fluorine and/or chlorine atoms. In another embodiment, the haloalkyl group is chosen from a C₁₋₄ haloalkyl group. Non-limiting exemplary haloalkyl groups include fluoromethyl, 2-fluoroethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

In the present disclosure, the term “hydroxyalkyl” as used by itself or as part of another group refers to an alkyl group substituted with one or more, e.g., one, two, or three, hydroxy groups. In one embodiment, the hydroxyalkyl group is a monohydroxyalkyl group, i.e., substituted with one hydroxy group. In another embodiment, the hydroxyalkyl group is a dihydroxyalkyl group, i.e., substituted with two hydroxy groups, e.g.,

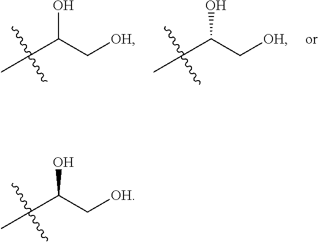 [SMILES: *C(O)CO ; *[C@H](O)CO ; "or" ; *[C@@H](O)CO]

In another embodiment, the hydroxyalkyl group is chosen from a C₁₋₄ hydroxyalkyl group. Non-limiting exemplary hydroxyalkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

In the present disclosure, the term “alkoxy” as used by itself or as part of another group refers to an optionally substituted alkyl, optionally substituted cycloalkyl, optionally substituted alkenyl or optionally substituted alkynyl attached to a terminal oxygen atom. In one embodiment, the alkoxy group is chosen from a C₁₋₄ alkoxy group. In another embodiment, the alkoxy group is chosen from a C₁₋₄ alkyl attached to a terminal oxygen atom, e.g., methoxy, ethoxy, and tert-butoxy.

In the present disclosure, the term “alkylthio” as used by itself or as part of another group refers to a sulfur atom substituted by an optionally substituted alkyl group. In one embodiment, the alkylthio group is chosen from a C₁₋₄ alkylthio group. Non-limiting exemplary alkylthio groups include —SCH₃, and —SCH₂CH₃.

In the present disclosure, the term “alkoxyalkyl” as used by itself or as part of another group refers to an alkyl group substituted with an alkoxy group. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

In the present disclosure, the term “haloalkoxy” as used by itself or as part of another group refers to a haloalkyl attached to a terminal oxygen atom. Non-limiting exemplary haloalkoxy groups include fluoromethoxy, difluoromethoxy, trifluoromethoxy, and 2,2,2-trifluoroethoxy.

In the present disclosure, the term “aryl” as used by itself or as part of another group refers to a monocyclic or bicyclic aromatic ring system having from six to fourteen carbon atoms (i.e., C₆-C₁₄ aryl). Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is chosen from phenyl or naphthyl.

In the present disclosure, the term “optionally substituted aryl” as used herein by itself or as part of another group means that the aryl as defined above is either unsubstituted or substituted with one to five substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl.

In one embodiment, the optionally substituted aryl is an optionally substituted phenyl. In one embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, and 3-chloro-4-fluorophenyl. The term optionally substituted aryl is meant to include groups having fused optionally substituted cycloalkyl and fused optionally substituted heterocyclo rings. Non-limiting examples include:

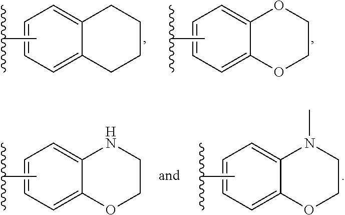

In the present disclosure, the term “phenylenyl” as used herein by itself or part of another group refers to a divalent form of an optionally substituted phenyl group. Non-limiting examples include:

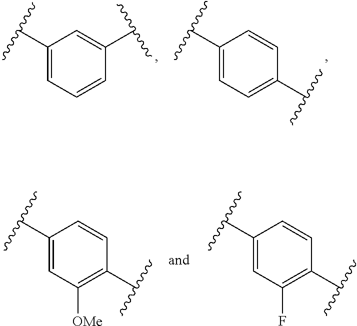 [SMILES: *c1cccc(*)c1 ; *c1ccc(*)cc1 ; *c1ccc(*)c(OC)c1 ; *c1ccc(*)c(F)c1]

In the present disclosure, the term “aryloxy” as used by itself or as part of another group refers to an optionally substituted aryl attached to a terminal oxygen atom. A non-limiting exemplary aryloxy group is PhO—.

In the present disclosure, the term “aralkyloxy” as used by itself or as part of another group refers to an aralkyl group attached to a terminal oxygen atom. A non-limiting exemplary aralkyloxy group is PhCH₂O—.

In the present disclosure, the term “heteroaryl” or “heteroaromatic” refers to monocyclic and bicyclic aromatic ring systems having 5 to 14 ring atoms (i.e., C₅-C₁₄ heteroaryl), wherein at least one carbon atom of one of the rings is replaced with a heteroatom independently selected from the group consisting of oxygen, nitrogen and sulfur. In one embodiment, the heteroaryl contains 1, 2, 3, or 4 heteroatoms independently selected from the group consisting of oxygen, nitrogen and sulfur. In one embodiment, the heteroaryl has three heteroatoms. In another embodiment, the heteroaryl has two heteroatoms. In another embodiment, the heteroaryl has one heteroatom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl), isoxazolyl (e.g., isoxazol-3-yl, isoxazol-4-yl, and isoxazol-5-yl), and indazolyl (e.g., 1H-indazol-3-yl). The term “heteroaryl” is also meant to include possible N-oxides. A non-limiting exemplary N-oxide is pyridyl N-oxide.

In one embodiment, the heteroaryl is a 5- or 6-membered heteroaryl. In one embodiment, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Non-limiting exemplary 5-membered heteroaryl groups include thienyl, furyl, pyrrolyl, oxazolyl, pyrazolyl, imidazolyl, thiazolyl, isothiazolyl, and isoxazolyl.

In another embodiment, the heteroaryl is a 6-membered heteroaryl, e.g., the heteroaryl is a monocyclic aromatic ring system having 6 ring atoms wherein at least one carbon atom of the ring is replaced with a nitrogen atom. Non-limiting exemplary 6-membered heteroaryl groups include pyridyl, pyrazinyl, pyrimidinyl, and pyridazinyl.

In the present disclosure, the term “optionally substituted heteroaryl” as used by itself or as part of another group means that the heteroaryl as defined above is either unsubstituted or substituted with one to four substituents, e.g., one or two substituents, independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. In one embodiment, the optionally substituted heteroaryl has one substituent. Any available carbon or nitrogen atom can be substituted. Non-limiting exemplary optionally substituted 5-membered heteroaryl groups include, but are not limited to

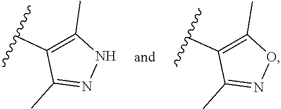

The term optionally substituted heteroaryl is also meant to include groups having fused optionally substituted cycloalkyl and fused optionally substituted heterocyclo rings. Non-limiting examples include:

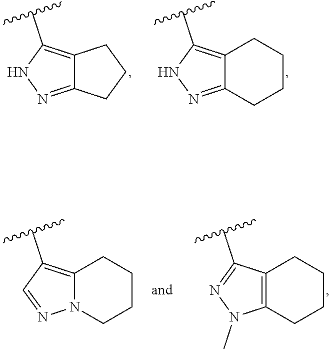 [SMILES: *c1[nH]nc2c1CCC2 ; *c1[nH]nc2c1CCCC2 ; *c1cnn2c1CCCC2 ; *c1nn(C)c2c1CCCC2]

In the present disclosure, the term “heteroarylenyl” as used herein by itself or part of another group refers to a divalent form of an optionally substituted heteroaryl group. In one embodiment, the heteroarylenyl is a 5-membered heteroarylenyl. Non-limiting examples of a 5-membered heteroarylenyl include:

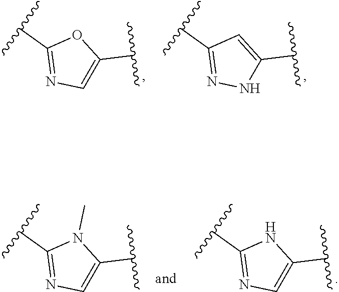

In one embodiment, the heteroarylenyl is a 6-membered heteroarylenyl. Non-limiting examples of a 6-membered heteroarylenyl include:

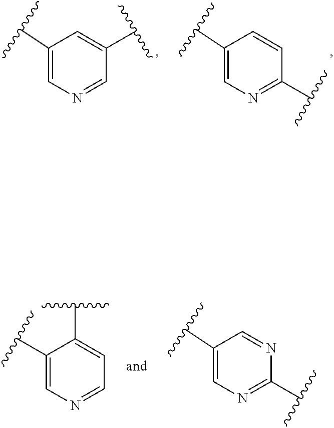 [SMILES: *c1cncc(*)c1 ; *c1ccc(*)nc1 ; *c1ccncc1* ; *c1cnc(*)nc1]

In the present disclosure, the term “heterocycle” or “heterocyclo” as used by itself or as part of another group refers to saturated and partially unsaturated (e.g., containing one or two double bonds) cyclic groups containing one, two, or three rings having from three to fourteen ring members (i.e., a 3- to 14-membered heterocyclo) wherein at least one carbon atom of one of the rings is replaced with a heteroatom. Each heteroatom is independently selected from the group consisting of oxygen, sulfur, including sulfoxide and sulfone, and/or nitrogen atoms, which can be oxidized or quaternized. The term “heterocyclo” is meant to include groups wherein a ring —CH₂— is replaced with a —C(═O)—, for example, cyclic ureido groups such as 2-imidazolidinone and cyclic amide groups such as β-lactam, γ-lactam, δ-lactam, ε-lactam, and piperazin-2-one. The term “heterocyclo” is also meant to include groups having fused optionally substituted aryl groups, e.g., indolinyl, chroman-4-yl. In one embodiment, the heterocyclo group is chosen from a 5- or 6-membered cyclic group containing one ring and one or two oxygen and/or nitrogen atoms. The heterocyclo can be optionally linked to the rest of the molecule through any available carbon or nitrogen atom. Non-limiting exemplary heterocyclo groups include dioxanyl, tetrahydropyranyl, 2-oxopyrrolidin-3-yl, piperazin-2-one, piperazine-2,6-dione, 2-imidazolidinone, piperidinyl, morpholinyl, piperazinyl, pyrrolidinyl, and indolinyl.

In the present disclosure, the term “optionally substituted heterocyclo” as used herein by itself or part of another group means the heterocyclo as defined above is either unsubstituted or substituted with one to four substituents independently selected from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, alkoxycarbonyl, CF₃C(═O)—, arylcarbonyl, alkylsulfonyl, arylsulfonyl, carboxy, carboxyalkyl, alkyl, optionally substituted cycloalkyl, alkenyl, alkynyl, optionally substituted aryl, optionally substituted heteroaryl, optionally substituted heterocyclo, alkoxyalkyl, (amino)alkyl, (carboxamido)alkyl, mercaptoalkyl, or (heterocyclo)alkyl. Substitution may occur on any available carbon or nitrogen atom, or both. Non-limiting exemplary optionally substituted heterocyclo groups include:

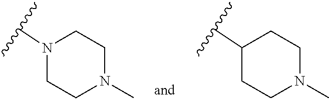

In the present disclosure, the term “amino” as used by itself or as part of another group refers to —NR^{10a}R^{10b}, wherein R^{10a} and R^{10b} are each independently hydrogen, alkyl, hydroxyalkyl, optionally substituted cycloalkyl, optionally substituted aryl, optionally substituted heterocyclo, or optionally substituted heteroaryl, or R^{10a} and R^{10b} are taken together to form a 3- to 8-membered optionally substituted heterocyclo. Non-limiting exemplary amino groups include —NH₂ and —N(H)(CH₃).

In the present disclosure, the term “(amino)alkyl” as used by itself or as part of another group refers to an alkyl group substituted with an amino group. Non-limiting exemplary amino alkyl groups include —CH₂CH₂NH₂, and —CH₂CH₂N(H)CH₃, —CH₂CH₂N(CH₃)₂, and —CH₂N(H)cyclopropyl.

In the present disclosure, the term “carboxamido” as used by itself or as part of another group refers to a radical of formula —C(═O)NR^{9a}R^{9b}, wherein R^{9a} and R^{9b} are each independently hydrogen, optionally substituted alkyl, hydroxyalkyl, optionally substituted cycloalkyl, optionally substituted aryl, optionally substituted heterocyclo, or optionally substituted heteroaryl, or R^{9a} and R^{9b} taken together with the nitrogen to which they are attached form a 3- to 8-membered optionally substituted heterocyclo group. In one embodiment, R^{9a} and R^{9b} are each independently hydrogen or optionally substituted alkyl. In one embodiment, R^{9a} and R^{9b} are taken together to taken together with the nitrogen to which they are attached form a 3- to 8-membered optionally substituted heterocyclo group. Non-limiting exemplary carboxamido groups include, but are not limited to, —CONH₂, —CON(H)CH₃, —CON(CH₃)₂, —CON(H)Ph,

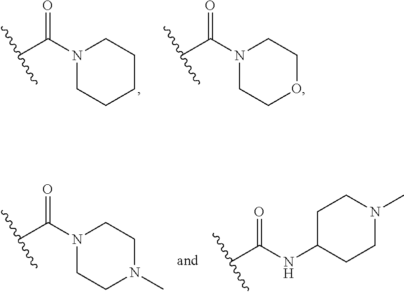

In the present disclosure, the term “sulfonamido” as used by itself or as part of another group refers to a radical of the formula —SO₂NR^{8a}R^{8b}, wherein R^{8a} and R^{8b} are each independently hydrogen, optionally substituted alkyl, or optionally substituted aryl, or R^{8a} and R^{8b} taken together with the nitrogen to which they are attached from a 3- to 8-membered heterocyclo group. Non-limiting exemplary sulfonamido groups include —SO₂NH₂, —SO₂N(H)CH₃, and —SO₂N(H)Ph.

In the present disclosure, the term “alkylcarbonyl” as used by itself or as part of another group refers to a carbonyl group, i.e., —C(═O)—, substituted by an alkyl group. A non-limiting exemplary alkylcarbonyl group is —COCH₃.

In the present disclosure, the term “arylcarbonyl” as used by itself or as part of another group refers to a carbonyl group, i.e., —C(═O)—, substituted by an optionally substituted aryl group. A non-limiting exemplary arylcarbonyl group is —COPh.

In the present disclosure, the term “alkoxycarbonyl” as used by itself or as part of another group refers to a carbonyl group, i.e., —C(═O)—, substituted by an alkoxy group. Non-limiting exemplary alkoxycarbonyl groups include —C(═O)OMe, —C(═O)OEt, and —C(═O)OtBu.

In the present disclosure, the term “alkylsulfonyl” as used by itself or as part of another group refers to a sulfonyl group, i.e., —SO₂—, substituted by any of the above-mentioned optionally substituted alkyl groups. A non-limiting exemplary alkylsulfonyl group is —SO₂CH₃.

In the present disclosure, the term “arylsulfonyl” as used by itself or as part of another group refers to a sulfonyl group, i.e., —SO₂—, substituted by any of the above-mentioned optionally substituted aryl groups. A non-limiting exemplary arylsulfonyl group is —SO₂Ph.

In the present disclosure, the term “mercaptoalkyl” as used by itself or as part of another group refers to any of the above-mentioned alkyl groups substituted by a —SH group.

In the present disclosure, the term “carboxy” as used by itself or as part of another group refers to a radical of the formula —COOH.

In the present disclosure, the term “carboxyalkyl” as used by itself or as part of another group refers to any of the above-mentioned alkyl groups substituted with a —COOH. A non-limiting exemplary carboxyalkyl group is —CH₂CO₂H.

In the present disclosure, the terms “aralkyl” or “arylalkyl” as used by themselves or as part of another group refers to an alkyl group substituted with one, two, or three optionally substituted aryl groups. In one embodiment, the optionally substituted aralkyl group is a C₁₋₄ alkyl substituted with one optionally substituted aryl group. In one embodiment, the optionally substituted aralkyl group is a Q or C₂ alkyl substituted with one optionally substituted aryl group. In one embodiment, the optionally substituted aralkyl group is a C₁ or C₂ alkyl substituted with one optionally substituted phenyl group. Non-limiting exemplary optionally substituted aralkyl groups include benzyl, phenethyl, —CHPh₂, —CH₂(4-F-Ph), —CH₂(4-Me-Ph), —CH₂(4-CF₃-Ph), and —CH(4-F-Ph)₂.

In the present disclosure, the terms “(heterocyclo)alkyl” as used by itself or part of another group refers to an alkyl group substituted with an optionally substituted heterocyclo group. In one embodiment, the (heterocyclo)alkyl is a C₁₋₄ alkyl substituted with one optionally substituted heterocyclo group. Non-limiting exemplary (heterocyclo)alkyl groups include:

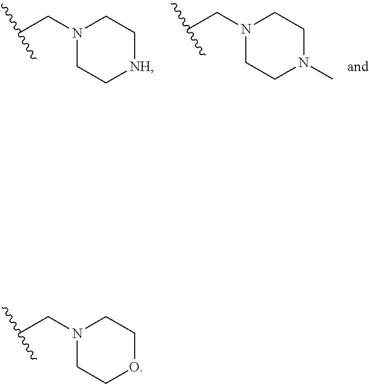

The present disclosure encompasses any of the Compounds of the Disclosure being isotopically-labelled, i.e., radiolabeled, by having one or more atoms replaced by an atom having a different atomic mass or mass number. Examples of isotopes that can be incorporated into Compounds of the Disclosure include isotopes of hydrogen, carbon, nitrogen, sulfur, oxygen, fluorine, and chlorine, such as ²H (or deuterium (D)), ³H, ¹¹C, ¹³C, ¹⁴C, ¹⁵N, ¹⁸O, ¹⁷O, ³⁵S, ¹⁸F, and ³⁶Cl, e.g., ²H, ³H, and ¹³C. In one embodiment, a portion of the atoms at a position within a Compound of the Disclosure are replaced, i.e., the Compound of the Disclosure is enriched at a position with an atom having a different atomic mass or mass number. In one embodiment, at least about 1% of the atoms are replaced with an atom having a different atomic mass or mass number. In another embodiment, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the atoms are replaced with an atom having a different atomic mass or mass number. Isotopically-labeled Compounds of the Disclosure can be prepared by methods known in the art.

### EXAMPLES

### Example 1

### Compound Synthesis and Characterization

### General Chemical Methods.

Unless otherwise noted, all purchased reagents were used as received without further purification. ¹H NMR and ¹³C NMR spectra were recorded on a Bruker Advance 400 MHz spectrometer. ¹H NMR spectra are reported in parts per million (ppm) downfield from tetramethylsilane (TMS). All ¹³C NMR spectra are reported in ppm and obtained with ¹H decoupling. In the spectral data reported, the format (δ) chemical shift (multiplicity, J values in Hz, integration) was used with the following abbreviations: s=singlet, d=doublet, t=triplet, q=quartet, m=multiplet. MS analyses were carried out with a Waters UPLC-mass spectrometer. The final compounds were all purified by C18 reverse phase preparative HPLC column with solvent A (0.1% TFA in H₂O) and solvent B (0.1% TFA in MeCN) as eluents. The purity of all the final compounds was determined to be >95% by UPLC-MS

The syntheses of the final compounds are outlined in Schemes 3-5. First, two key common intermediates 53 and 58 were synthesized as shown in Schemes 1 and 2, respectively. The commercial 4-acetoxybenzoic acid (49) was converted to the acyl chloride which, after Friedel-Crafts acylation of commercial 6-methoxy-2-(4-methoxyphenyl)benzo-[b]thiophene furnished compound 50. Deacetylation of 50 under aqueous basic conditions gave compound 51. This was converted to the alkyl bromide, which was substituted with excess ethylamine to afford the secondary amine (52). Cleavage of both aryl methoxy ethers in 52 with boron tribromide furnished the dihydroxy intermediate 53. Following a published procedure,⁴⁷ the synthesis of compound 58 commenced with the tert-butyloxycarbonyl protection of commercial (S)-1-(4-bromophenyl)ethan-1-amine (54). Subsequent Suzuki coupling of 54 with 4-methylthiazole afforded compound 55, and this was followed by deprotection under acidic conditions and amide coupling with commercially available (2S,4R)-1-(tert-butoxycarbonyl)-4-hydroxypyrrolidine-2-carboxylic acid to give 56, which was deprotected under the same conditions, then subjected to amide coupling with commercially available (S)-1-(tert-butoxycarbonyl)piperidine-2-carboxylic acid to afford compound 57, which after acidic deprotection afforded compound 58.

As shown in Scheme 3, the synthesis of compound 12 commenced with the mesylation of commercial 2-(2-(2-(prop-2-yn-1-yloxy)ethoxy)ethoxy)ethan-1-ol (59a) to compound 60a using methanesulfonyl chloride with trimethylamine as base. Nucleophilic substitution of 60a with compound 53 under mild basic conditions afforded the N-substituted compound (61a). Sonogashira coupling of compound 61a with the previously published compound 3-(4-iodo-1-oxoisoindolin-2-yl)piperidine-2,6-dione⁵⁰ afforded compound 12 in high yield. Compound 13 was synthesized using the procedure described for the synthesis of compound 12 starting from oct-7-yn-1-ol (59b).

As shown in Scheme 4, study of diverse linkers commenced with the preparation of compounds 63 or 65, which are commercially available and can be prepared from 62 or 64, respectively The substitution reaction of compound 63 or 65 with compound 53 furnished compound 66, which upon acidic deprotection gave the acid (67). Amide coupling of compounds 67 and 58 afforded the final compounds 14-21 and 30-37 in high yields.

As shown in Scheme 5, the intermediate 51 was used for the SAR studies of the N-substituent groups. Compound 51 was first converted to the corresponding alkyl bromide which, subjected nucleophilic attack with excess of the primary amine furnished compound 68. The substitution reaction of compound 68 with tert-butyl 8-bromooctanoate (65) furnished the linker-attached intermediate, which underwent boron tribromide-mediated demethylation and deprotection to afford the acid (69). Amide coupling between compounds 69 and 58 afforded the final compounds 22-29 in high yields. Compounds 38-48 were synthesized using the general procedure that was used to prepare compound 15.

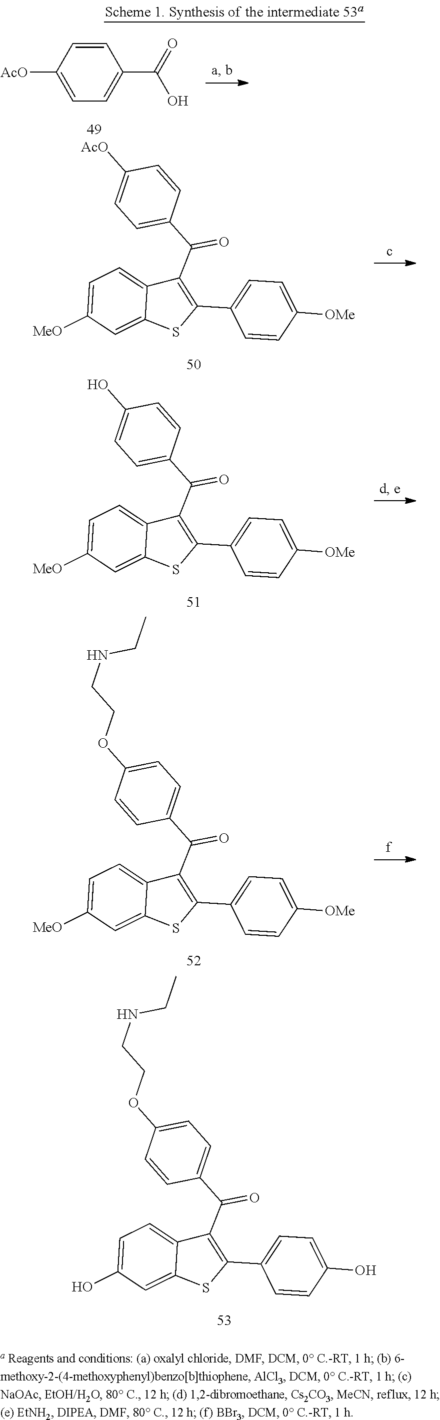

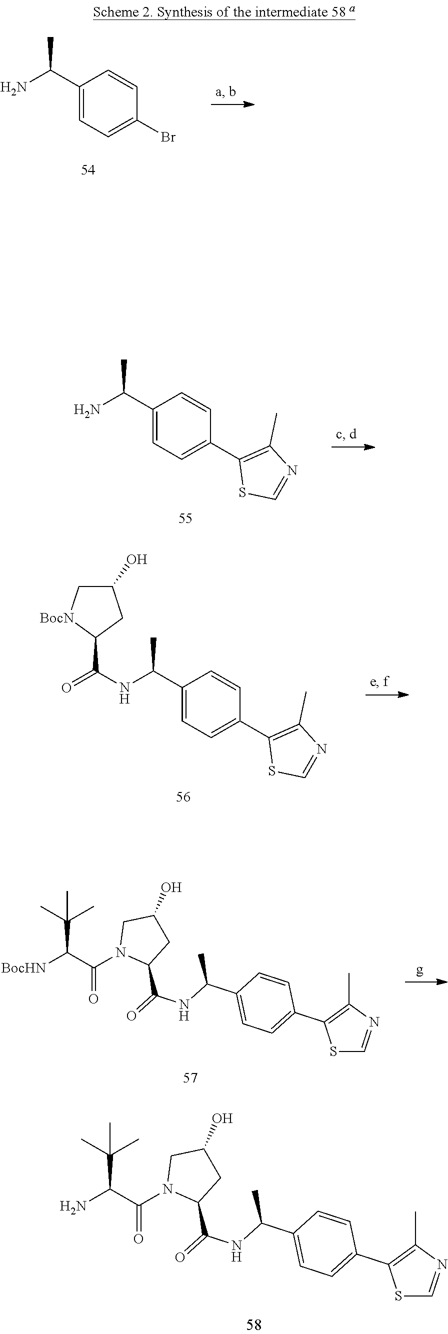

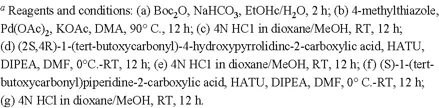

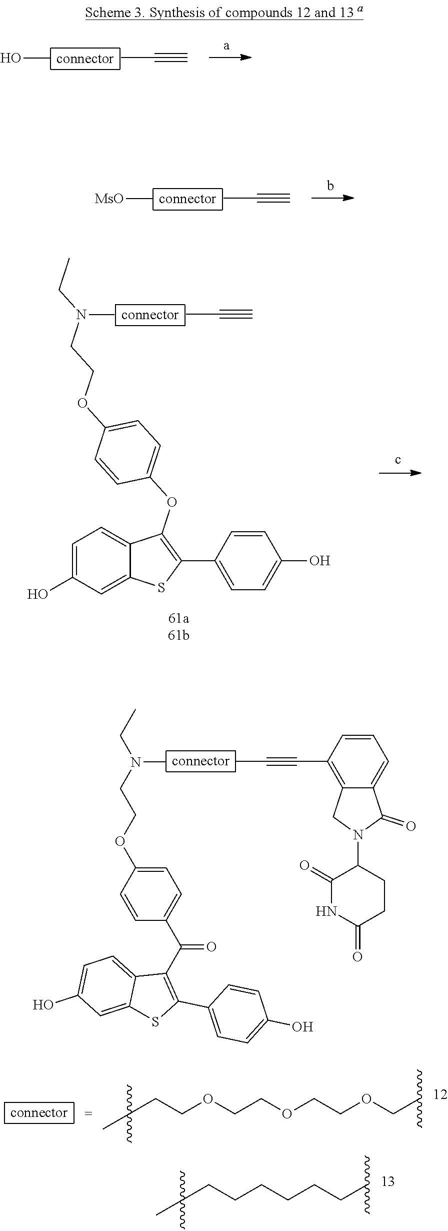

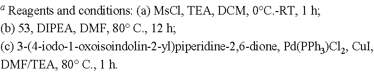

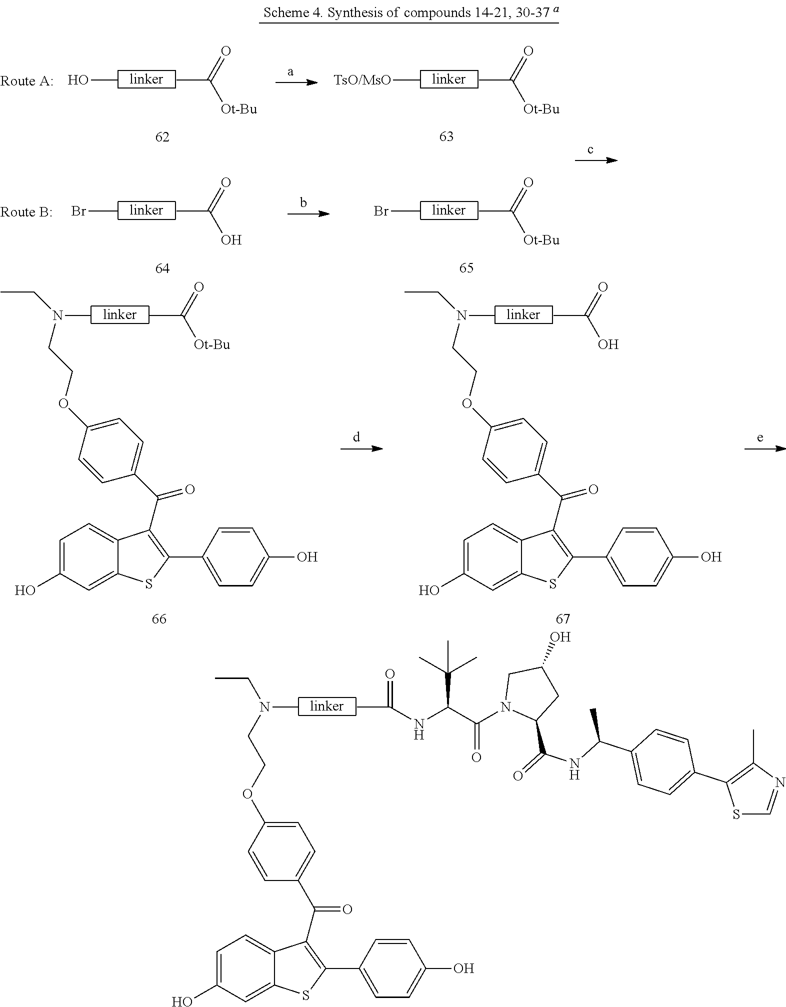

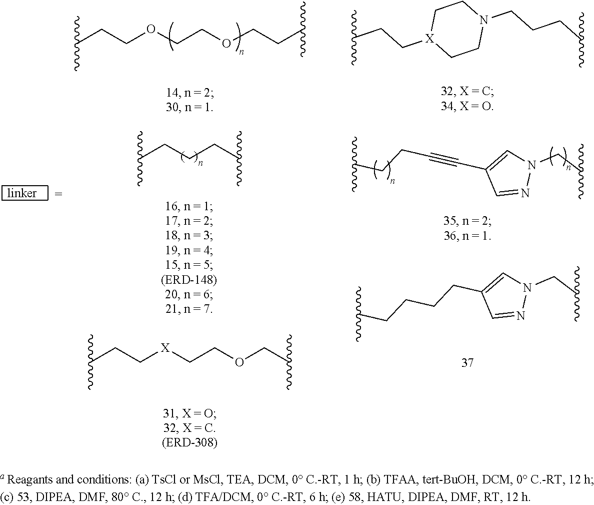

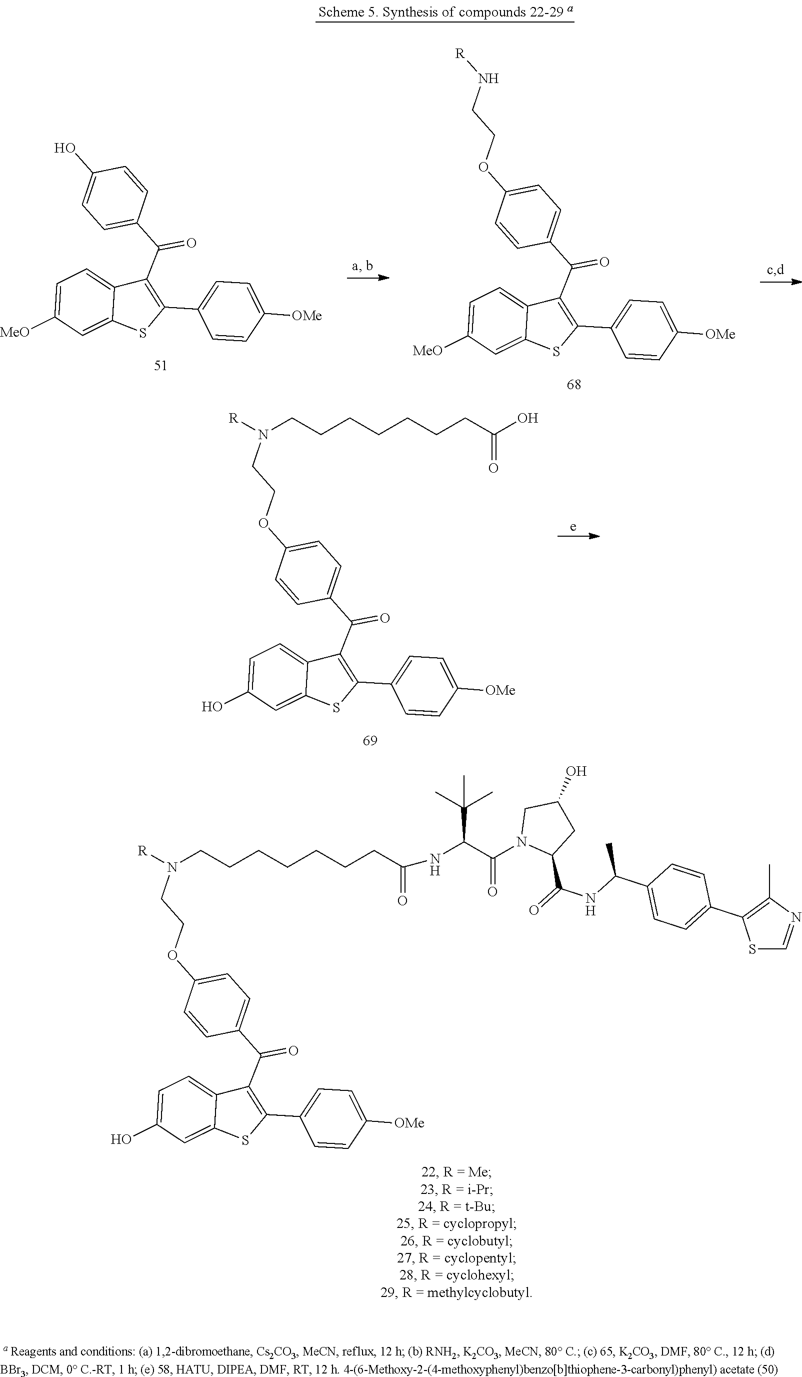

Oxalyl chloride (9.70 mL, 120 mmol, 3.0 eq) was added dropwise under N₂ to a solution of 4-acetoxybenzoic acid (49) (7.206 g, 40 mmol, 1.0 eq) in anhydrous DCM (80 mL) at 0° C. Then several drops of DMF were added. The solution was warmed to rt and stirred for 1 h. The solution was concentrated and dried to obtain the acyl chloride as a white solid. This intermediate was dissolved in anhydrous DCM (150 mL), then 6-methoxy-2-(4-methoxyphenyl)-benzo[b]thiophene (8.65 g, 32 mmol, 0.8 eq) was added followed by addition of AlCl₃ (8.00 g, 60 mmol, 1.5 eq) in three portions over a period of 5 min with vigorous stirring at 0° C. under N₂. The mixture was warmed to rt and stirred for 1 h. The reaction was quenched by slow addition of ice-H₂O followed by 1N HCl (aq). The layers were separated and the aqueous layer was extracted twice with DCM. The combined organic layer was dried over anhydrous Na₂SO₄. After filtration and concentration, the residue was purified on a silica gel flash column with hexane:DCM (100:1-1:100) to afford the intermediate (50) as a yellow solid (5.517 g, 40% yield). ¹H NMR (CDCl₃, 400 MHz) δ (ppm) 7.81 (d, J=8.8 Hz, 2H), 7.61 (d, J=8.8 Hz, 1H), 7.32-7.29 (m, 3H), 7.02-6.99 (m, 3H), 6.74 (d, J=8.8 Hz, 2H), 3.86 (s, 3H), 3.73 (s, 3H), 2.25 (s, 3H); ¹³C NMR (CDCl₃, 100 MHz) δ (ppm) 193.15, 168.63, 159.99, 157.78, 154.38, 144.16, 140.10, 135.03, 133.76, 131.52, 130.48, 130.02, 125.76, 124.16, 121.54, 114.99, 114.13, 104.54, 55.65, 55.28, 21.16; UPLC-MS (ESP) calc. for C₂₅H₂₁O₅S [M+1]⁺: 433.11, found 433.37.

### (4-Hydroxyphenyl)(6-methoxy-2-(4-methoxyphenyl)benzo[b]thiophen-3-yl)methanone (51)

Compound 50 (5.517 g, 12.76 mmol, 1.0 eq) was dissolved in EtOH (70 mL) and H₂O (30 mL). Then NaOAc (5.23 g, 63.8 mmol, 5.0 eq) was added. The solution was stirred at 90-100° C. for 12 h. The solution was then cooled to rt and concentrated. The residue was diluted in EtOAc and H₂O. The organic layer was separated and the aqueous layer was extracted twice with EtOAc. The combined organic layer was dried over anhydrous Na₂SO₄. After filtration and concentration, the residue was purified by silica gel flash column chromatography with hexane:EtOAc (5:1-2:1) to afford intermediate 51 as yellow oil (4.7 g, 95% yield). ¹H NMR (CD₃OD, 400 MHz) δ (ppm) 7.64 (d, J=9.2 Hz, 2H), 7.43 (d, J=8.8 Hz, 1H), 7.30 (d, J=2.4 Hz, 1H), 7.24 (d, J=8.8 Hz, 2H), 6.89 (dd, J=8.8 Hz, J=2.4 Hz, 1H), 6.69-6.64 (m, 4H), 3.73 (s, 3H), 3.59 (s, 3H); ¹³C NMR (CD₃OD, 100 MHz) δ (ppm) 193.95, 162.85, 159.92, 157.81, 142.33, 140.08, 133.78, 132.55, 130.34, 129.91, 129.06, 125.78, 123.43, 115.04, 114.63, 113.79, 104.34, 54.78, 54.39; UPLC-MS (ESI⁺) calc. for C₂₃H₁₉O₄S [M+1]⁺: 391.10, found 391.42.

### (4-(2-(Ethylamino)ethoxy)phenyl)(6-methoxy-2-(4-methoxyphenyl)benzo[b]thiophen-3-yl)methanone (52)

1,2-dibromoethane (2.0 mL, 24.0 mmol, 2.0 eq) and Cs₂CO₃ (5.86 g, 18.0 mmol, 1.5 eq) were added sequentially to a solution of compound 51 (4.7 g, 12.0 mmol, 1.0 eq) in MeCN (200 mL). The solution was heated to reflux for 12 h. The solution was filtered and the precipitate was washed with MeCN. The concentrated residue was used in the next step without further column purification. EtNH₂ (2.0 M in THF) (60 mL, 120 mmol, 10.0 eq) was added to a solution of the residue in DMF. The solution was heated to 80° C. and stirred for 12 h. After cooling to rt, the reaction mixture was diluted in EtOAc and saturated brine. The aqueous layer was extracted with EtOAc twice. The combined organic layer was dried and concentrated. The residue was purified by silica gel flash column chromatography with DCM:MeOH (10:1) to afford compound 52 as a yellow solid (4.43 g, 80% yield). ¹H NMR (CD₃OD, 400 MHz) δ (ppm) 7.63 (d, J=8.8 Hz, 2H), 7.42 (d, J=8.8 Hz, 1H), 7.32 (d, J=2.4 Hz, 1H), 7.20 (d, J=8.8 Hz, 2H), 6.88 (dd, J=8.8 Hz, J=2.4 Hz, 1H), 6.71 (d, J=8.8 Hz, 2H), 6.64 (d, J=8.8 Hz, 2H), 3.93 (t, J=4.2 Hz, 2H), 3.75 (s, 3H), 3.59 (s, 3H), 2.83 (t, J=5.2 Hz, 2H), 2.59 (q, J=7.2 Hz, 2H), 1.06 (t, J=7.2 Hz, 3H); ¹³C NMR (CD₃OD, 100 MHz) δ (ppm) 194.88, 164.48, 161.27, 159.20, 143.95, 141.41, 135.02, 133.39, 131.61, 131.48, 131.26, 127.01, 124.76, 115.97, 115.30, 115.10, 105.67, 68.13, 56.10, 55.70, 49.65, 44.52, 14.68; UPLC-MS (ESI⁺) calc. for C₂₇H₂₈NO₄S [M+1]⁺: 462.17, found 462.27.

### (4-(2-(Ethylamino)ethoxy)phenyl)(6-hydroxy-2-(4-hydroxyphenyl)benzo[b]thiophen-3-yl)methanone (53)

8.0 mL of a solution of BBr₃ (1.0 M in DCM) (8.0 mmol, 4.0 eq) was slowly added under N₂ to a solution of 52 (923 mg, 2.0 mmol, 1.0 eq) in anhydrous DCM (30 mL) at 0° C. The dark-red solution was stirred at rt for 2 h, then MeOH (1.0 mL) was added dropwise to quench the reaction. The solution was concentrated and the residue was dissolved in EtOAc (50 mL), then aqueous saturated NaHCO₃ (50 mL) and EtOH (5 mL) were added. The organic layer was separated and dried over anhydrous Na₂SO₄. After filtration, the solution was concentrated and the residue was purified by silica gel flash column chromatography with DCM:MeOH (10:1-5:1) to afford the intermediate (53) as a yellow solid (520 mg, 60% yield). ¹H NMR (CD₃OD, 400 MHz) δ (ppm) 7.72 (d, J=8.8 Hz, 2H), 7.43 (d, J=8.8 Hz, 1H), 7.27 (d, J=2.0 Hz, 1H), 7.17 (d, J=8.8 Hz, 2H), 6.91 (d, J=8.8 Hz, 2H), 6.87 (dd, J=8.8 Hz, J=2.4 Hz, 1H), 6.61 (d, J=8.8 Hz, 2H), 4.27 (t, J=4.8 Hz, 2H), 3.42 (t, J=4.8 Hz, 2H), 3.14 (q, J=7.2 Hz, 2H), 1.33 (t, J=7.2 Hz, 3H); ¹³C NMR (CD₃OD, 100 MHz) δ (ppm) 195.41, 163.41, 159.22, 156.80, 144.30, 141.45, 134.21, 133.45, 132.55, 131.42, 131.00, 125.99, 124.68, 116.43, 116.08, 115.39, 107.90, 64.68, 47.41, 44.36, 11.40; UPLC-MS (ESP) calc. for C₂₅H₂₄NO₄S [M+1]⁺: 434.14, found 434.11.

### tert-Butyl (2S,4R)-4-hydroxy-2-(((S)-1-(4-(4-methylthiazol-5-yl)phenyl)ethyl)carbamoyl)-pyrrolidine-1-carboxylate (56)

Compound 55, synthesized using a reported procedure⁴⁷ was dissolved in 4N HCl in dioxane (25 mL, 100 mmol) and MeOH (25 mL) and the mixture was stirred at ambient temperature for 12 h. The mixture was concentrated and the residue was dried under vacuum to afford the intermediate, which was used in next step without further purification.

HATU (14.51 g, 38.2 mmol, 1.2 eq) was added to a solution of the intermediate (55) obtained as described above (6.95 g, 31.8 mmol, 1.0 eq), (2S,4R)-1-(tert-butoxycarbonyl)-4-hydroxy-pyrrolidine-2-carboxylic acid (7.36 g, 31.8 mmol, 1.0 eq), and DIPEA (11.08 mL, 63.6 mmol, 2.0 eq) in DMF (36 mL) at 0° C. under N₂. The mixture was stirred at ambient temperature for 12 h when TLC showed that the reaction was complete. The reaction mixture was quenched with H₂O (200 mL) and extracted with EtOAc (150 mL×2). The combined organic layer was washed with brine (200 mL) and dried over Na₂SO₄. The organic solution was filtered and concentrated and the residue was purified by silica gel flash column chromatography with hexane:EtOAc (100:1-1:100), then DCM:MeOH (10:1) to afford the intermediate (56) as white solid (10.98 g, 80% yield). ¹H NMR (CD₃OD, 400 MHz) δ (ppm) 8.84 (s, 1H), 7.43-7.37 (m, 4H), 5.11-5.07 (m, 1H), 4.44-4.37 (m, 2H), 3.60-3.46 (m, 2H), 2.44 (s, 3H), 2.27-2.22 (m, 1H), 1.98-1.91 (m, 1H), 1.50 (d, J=7.2 Hz, 3H), 1.46 (s, 9H); UPLC-MS (ESI⁺) calc. for C₂₂H₃₀N₃O₄S [M+1]⁺: 432.20, found 432.20.

### tert-Butyl ((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamate (57)

This solid (56), obtained as described above was dissolved in 4N HCl in dioxane (25 mL, 100 mmol) and MeOH (25 mL) and the mixture was stirred at ambient temperature for 12 h. The mixture was then concentrated and the residue was dried under vacuum to afford an intermediate, which was used in next step without further purification. UPLC-MS (ESI⁺) calc. for C₁₇H₂₂N₃O₂S [M+1]⁺: 332.14, found 332.11. HATU (1.37 g, 3.6 mmol, 1.2 eq) was added to a solution of this intermediate (994 mg, 3.0 mmol, 1.0 eq), (S)-2-((tert-butoxycarbonyl)amino)-3,3-dimethylbutanoic acid (694 mg, 3.0 mmol, 1.0 eq), and DIPEA (1.57 mL, 9.0 mmol, 3.0 eq) in DMF (10 mL) at 0° C. under N₂. The mixture was stirred at ambient temperature for 12 h when TLC showed that the reaction was complete. The reaction mixture was quenched with H₂O (100 mL) and extracted with EtOAc (75 mL×2). The combined organic layer was washed with brine (100 mL) and dried over Na₂SO₄. The organic solution was filtered and concentrated. The residue was purified by silica gel flash column chromatography with hexane:EtOAc then DCM:MeOH to afford the desired compound (57) as a white solid (1.31 g, 80% yield). ¹H NMR (CDCl₃, 400 MHz) δ (ppm) 8.65 (s, 1H), 7.70 (d, J=8.0 Hz, 1H), 7.35-7.31 (m, 4H), 5.29 (d, J=9.2 Hz, 1H), 5.06-5.01 (m, 1H), 4.67 (t, J=8.0 Hz, 1H), 4.46-4.44 (m, 1H), 4.22-4.19 (m, 1H), 3.91 (d, J=17.2 Hz, 1H), 3.61-3.58 (m, 1H), 2.46 (s, 3H), 2.37-2.30 (m, 1H), 2.04-1.99 (m, 1H), 1.44 (d, J=7.2 Hz, 3H), 1.35 (s, 9H), 0.96 (s, 9H); ¹³C NMR (CDCl₃, 100 MHz) δ (ppm) 172.22, 170.13, 156.15, 150.56, 148.21, 143.43, 131.74, 130.59, 129.49, 126.46, 80.18, 69.91, 58.86, 56.58, 48.74, 38.60, 36.02, 35.48, 28.34, 26.39, 22.17, 15.95; UPLC-MS (ESI⁺) calc. for C₂₈H₄₁N₄O₅S [M+1]⁺: 545.28, found 545.35.

### (2S,4R)-1-((S)-2-Amino-3,3-dimethylbutanoyl)-4-hydroxy-N—((S)-1-(4-(4-methylthiazol-5-yl)phenyl)ethyl)pyrrolidine-2-carboxamide (58)

The solid material (57) obtained as described above was dissolved in 4N HCl in dioxane (4 mL, 16 mmol) and MeOH (4.0 mL) and the mixture was stirred at ambient temperature for 12 h. The mixture was then concentrated and the residue was dried under vacuum to afford the crude product, which was purified by reversed-phase preparative HPLC to afford the pure final compound (58) as an off-white solid. UPLC-MS (ESI⁺) calc. for C₂₃H₃₃N₄O₃S [M+1]⁺: 445.23, found 445.44.

### (2S,4R)-1-((S)-2-Acetamido-3,3-dimethylbutanoyl)-4-hydroxy-N—((S)-1-(4-(4-methylthiazol-5-yl)phenyl)ethyl)pyrrolidine-2-carboxamide (11)

HATU (21 mg, 0.055 mmol, 1.1 eq) was added to a mixture of compound 65 (23 mg, 0.05 mmol, 1.0 eq), AcOH (4 μL, 0.06 mmol, 1.2 eq), and DIPEA (26 μL, 0.15 mmol, 3.0 eq) in DMF (2 mL) at 0° C. under N₂. The mixture was stirred at ambient temperature for 1 h, then the crude mixture was purified by reversed-phase preparative HPLC to afford the title compound as a white solid (19 mg, 80% yield). ¹H NMR (CD₃OD, 400 MHz) δ (ppm) 9.02 (s, 1H), 7.47-7.42 (m, 4H), 5.04-4.98 (m, 1H), 4.62-4.55 (m, 2H), 4.43-4.41 (m, 1H), 3.88 (d, J=1.08 Hz, 1H), 3.74 (dd, J=10.8 Hz, J=4.0 Hz, 1H), 2.50 (s, 3H), 2.22-2.16 (m, 1H), 2.00 (s, 3H), 1.98-1.91 (m, 1H), 1.51 (d, J=6.8 Hz, 3H), 1.05 (s, 9H); ¹³C NMR (CD₃OD, 100 MHz) δ (ppm) 173.26, 173.11, 172.28, 153.34, 148.20, 146.01, 133.91, 131.04, 130.51, 127.69, 127.52, 70.97, 60.55, 59.22, 57.97, 50.14, 38.77, 36.41, 26.99, 22.38, 22.29, 15.41; UPLC-MS (ESI⁺) calc. for C₂₅H₃₅N₄O₄S [M+1]⁺: 487.24, found 487.43; Purity 98.5% (HPLC).

### 3-(4-(3-Ethyl-1-(4-(6-hydroxy-2-(4-hydroxyphenyl)benzo[b]thiophene-3-carbonyl)phenoxy)-6,9,12-trioxa-3-azapentadec-14-yn-15-yl)-1-oxoisoindolin-2-yl)piperidine-2,6-dione (12)

Methanesulfonyl chloride (0.35 mL, 4.5 mmol, 1.5 eq) and Et₃N (0.84 mL, 6.0 mmol, 2.0 eq) were added sequentially to a solution of the commercial compound 2-(2-(2-(prop-2-yn-1-yloxy)ethoxy)ethoxy)ethan-1-ol (59a) (565 mg, 3.0 mmol, 1.0 eq) in DCM (10 mL) at 0° C. The mixture was warmed to rt and stirred for 1 h. After concentration, the residue was purified by silica gel flash column chromatography with hexane:EtOAc (2:1-1:2) to afford the title compound (60a) as a colorless oil (710 mg, 89% yield). ¹H NMR (CDCl₃, 400 MHz) δ (ppm) 4.21-4.18 (m, 2H), 4.01 (d, J=2.4 Hz, 2H), 3.61-3.58 (m, 2H), 3.51-3.46 (m, 8H), 2.92 (s, 3H), 2.41 (t, J=2.4 Hz, 1H); ¹³C NMR (CDCl₃, 100 MHz) δ (ppm) 79.71, 74.91, 70.33, 70.25, 70.09, 69.51, 68.90, 68.77, 58.09, 37.43.

DIPEA (0.09 mL, 0.5 mmol, 5.0 eq) was added to a solution of compound 53 (44 mg, 0.1 mmol, 1.0 eq) and 60a (40 mg, 0.15 mmol, 1.5 eq) in DMF (3.0 mL). The solution was stirred at 100° C. for 12 h. After cooling to rt, the residue was purified by reversed-phase preparative HPLC to afford the title compound (61a) as a white solid (30 mg, 50% yield). UPLC-MS (ESI⁺) calc. for C₃₄H₃₈NO₇S [M+1]⁺: 604.24, found 604.30.

3-(4-Iodo-1-oxoisoindolin-2-yl)piperidine-2,6-dione (19 mg, 0.05 mmol, 1.0 eq) was added to a solution of compound 61a (30 mg, 0.05 mmol, 1.0 eq) in DMF (2.0 mL). The solution was purged and refilled with N₂ three times with sonication then Pd(PPh₃)₂Cl₂ (3.5 mg, 0.005 mmol, 0.1 eq), CuI (2.0 mg, 0.01 mmol, 0.2 eq) and Me₃N (2.0 mL) were added sequentially. The solution was purged and refilled with N₂. The solution was stirred at 80° C. for 1 h and was then cooled to rt. EtOAc and H₂O were added and the aqueous layer was extracted with EtOAc twice. The combined organic layer was dried over anhydrous Na₂SO₄. After filtration and concentration, the residue was purified by reversed-phase preparative HPLC to afford the title compound (12) as a yellow solid (18 mg, 43% yield). ¹H NMR (CD₃OD, 400 MHz) δ (ppm) 7.76-7.71 (m, 3H), 7.58 (d, J=7.6 Hz, 1H), 7.46-7.40 (m, 2H), 7.25 (d, J=2.0 Hz, 1H), 7.16 (d, J=8.8 Hz, 2H), 6.89 (d, J=8.8 Hz, 2H), 6.85 (dd, J=8.8 Hz, J=2.0 Hz, 1H), 6.60 (d, J=8.8 Hz, 2H), 5.12 (dd, J=13.2 Hz, J=5.2 Hz, 1H), 4.42-4.34 (m, 6H), 3.81-3.78 (m, 2H), 3.70-3.59 (m, 10H), 3.43 (t, J=4.8 Hz, 2H), 3.35 (q, J=7.6 Hz, 2H), 2.90-2.81 (m, 1H), 2.74-2.68 (m, 1H), 2.37-2.30 (m, 1H), 2.11-2.05 (m, 1H), 1.32 (t, J=7.6 Hz, 3H); ¹³C NMR (CD₃OD, 100 MHz) δ (ppm) 195.30, 174.53, 172.14, 170.68, 163.12, 159.25, 156.85, 145.48, 144.12, 141.47, 135.99, 134.20, 133.51, 133.10, 132.62, 131.44, 130.98, 129.77, 126.01, 124.75, 124.71, 119.34, 116.50, 116.12, 115.49, 107.94, 91.94, 82.61, 71.34, 71.24, 70.43, 65.49, 63.61, 59.69, 54.01, 53.58, 53.13, 51.37, 32.30, 24.02, 9.07; UPLC-MS (ESI⁺) calc. for C₄₇H₄₈N₃O₁₀S [M+1]⁺: 846.31, found 846.52; Purity 99.1% (HPLC).

### 3-(4-(8-(Ethyl(2-(4-(6-hydroxy-2-(4-hydroxyphenyl)benzo[b]thiophene-3-carbonyl)-phenoxy)ethyl)amino)oct-1-yn-1-yl)-1-oxoisoindolin-2-yl)piperidine-2,6-dione (13)

This compound was prepared from 59b using a three-step procedure similar to that used for compound 12. ¹H NMR (CD₃OD, 400 MHz) δ (ppm) 7.73-7.71 (m, 3H), 7.56 (d, J=7.2 Hz, 1H), 7.47-7.42 (m, 2H), 7.26 (d, J=2.0 Hz, 1H), 7.16 (d, J=8.4 Hz, 2H), 6.91-6.86 (m, 3H), 6.60 (d, J=8.8 Hz, 2H), 5.16 (dd, J=13.6 Hz, J=5.2 Hz, 1H), 4.50 (d, J=17.2 Hz, 1H), 4.44 (d, J=17.2 Hz, 1H), 4.35 (t, J=4.4 Hz, 2H), 3.58 (t, J=4.4 Hz, 2H), 3.32-3.30 (m, 2H), 3.21 (q, J=7.6 Hz, 2H), 2.93-2.84 (m, 1H), 2.77-2.73 (m, 1H), 2.50-2.44 (m, 3H), 2.17-2.12 (m, 1H), 1.79-1.72 (m, 2H), 1.68-1.61 (m, 2H), 1.58-1.51 (m, 2H), 1.47-1.42 (m, 2H), 1.33 (t, J=7.2 Hz, 3H); ¹³C NMR (CD₃OD, 100 MHz) δ (ppm) 195.28, 174.58, 172.22, 170.98, 163.03, 159.27, 156.87, 145.24, 144.38, 141.48, 135.77, 134.20, 133.51, 132.92, 132.75, 131.47, 130.99, 129.62, 126.02, 124.72, 123.74, 120.90, 116.46, 116.12, 115.38, 107.92, 97.08, 77.41, 63.59, 54.42, 53.66, 52.53, 50.31, 32.33, 29.39, 27.05, 24.68, 24.08, 19.94, 9.10; UPLC-MS (ESI⁺) calc. for C₄₆H₄₆N₃O₇S [M+1]⁺: 784.31, found 784.27; Purity 98.9% (HPLC).

General Procedure for Preparation of ER PROTACs as described in Scheme 4

Route A: exemplified by compound 32 (ERD-308).

### (2S,4R)-1-((S)-2-(2-((5-(Ethyl(2-(4-(6-hydroxy-2-(4-hydroxyphenyl)benzo[b]thiophene-3-carbonyl)phenoxy)ethyl)amino)pentyl)oxy)acetamido)-3,3-dimethylbutanoyl)-4-hydroxy-N—((S)-1-(4-(4-methylthiazol-5-yl)phenyl)ethyl)pyrrolidine-2-carboxamide (32, ERD-308)

NaOH (4.0 g, 100.0 mmol, 10.0 eq) and tetrabutyl ammonium chloride (2.78 g, 10.0 mmol, 1.0 eq) were added sequentially to a solution of 5-(benzyloxy)pentan-1-ol (1.94 g, 10.0 mmol, 1.0 eq) and tert-butyl 2-bromoacetate (3.90 g, 20.0 mmol, 2.0 eq) in H₂O (20 mL) and DCM (20 mL). The solution was stirred vigorously at rt overnight until TLC showed that the reaction was complete. The mixture was partitioned between DCM (100 mL) and H₂O (100 mL) and the organic layer was collected, washed with brine (100 mL), dried over anhydrous Na₂SO₄, and concentrated under reduced pressure to give a residue that was purified by silica gel flash column chromatography with hexane:EtOAc (10:1-5:1) to afford tert-butyl 2-((5-(benzyloxy)pentyl)-oxy)acetate as a colorless oil (987 mg, 32% yield).

A mixture of ten-butyl 2-((5-(benzyloxy)pentyl)oxy)acetate (770 mg, 2.5 mmol, 1.0 eq) and 10 wt % palladium on carbon (100 mg) in MeOH (20 mL) was stirred at rt overnight under an H₂ atmosphere. When TLC showed that the reaction was complete, the solution was filtered through celite and washed with MeOH. The combined filtrate was concentrated under reduced pressure. The residue was purified by silica gel flash column chromatography with hexane:EtOAc (2:1-1:1) to afford tert-butyl 2-((5-hydroxypentyl)oxy)acetate (62) as a colorless oil (671 mg, 95% yield). ¹H NMR (CDCl₃, 400 MHz) δ (ppm) 3.58 (s, 2H), 3.21 (t, J=6.8 Hz, 2H), 3.15 (t, J=6.8 Hz, 2H), 1.31-1.14 (m, 4H), 1.12-1.05 (m, 12H); ¹³C NMR (CDCl₃, 100 MHz) δ (ppm) 169.51, 80.94, 71.18, 68.20, 61.54, 31.96, 28.97, 27.67, 21.95.

4-Toluenesulfonyl chloride (879 mg, 4.6 mmol, 1.5 eq) and Et₃N (0.86 mL, 6.14 mmol, 2.0 eq) were added sequentially to a solution of tert-butyl 2-((5-hydroxypentyl)oxy)acetate (62) (671 mg, 3.07 mmol, 1.0 eq) in DCM (10 mL) at 0° C. The mixture was warmed to rt and stirred for 1 h. After concentration, the residue was purified by silica gel flash column chromatography with hexane:EtOAc (5:1-2:1) to afford the intermediate tert-butyl 2-((5-(tosyloxy)pentyl)oxy)acetate (63) as a colorless oil (1.02 g, 89% yield). ¹H NMR (CDCl₃, 400 MHz) δ (ppm) 7.75 (d, J=8.0 Hz, 2H), 7.32 (d, J=8.0 Hz, 2H), 3.99 (t, J=6.4 Hz, 2H), 3.88 (s, 2H), 3.43 (t, J=6.4 Hz, 2H), 2.42 (s, 3H), 1.68-1.61 (m, 2H), 1.57-1.50 (m, 2H), 1.44 (s, 9H), 1.42-1.36 (m, 2H); ¹³C NMR (CDCl₃, 100 MHz) δ (ppm) 169.76, 144.75, 133.16, 129.89, 127.92, 81.54, 71.19, 70.55, 68.76, 28.99, 28.65, 28.15, 22.07, 21.67; UPLC-MS (ESI⁺) calc. for C₁₈H₂₈NaO₆S [M+23]⁺: 395.15, found 395.36.

DIPEA (0.18 mL, 1.0 mmol, 5.0 eq) was added to a solution of compound 53 (87 mg, 0.2 mmol, 1.0 eq) and tert-butyl 2-((5-(tosyloxy)pentyl)oxy)acetate 63 (223 mg, 0.6 mmol, 3.0 eq) in DMF (3.0 mL). The solution was stirred at 80° C. for 12 h. After cooling to rt, the solution was diluted with EtOAc and H₂O. The organic layer was separated and dried over anhydrous Na₂SO₄. After filtration and concentration, the residue was purified by silica gel flash column chromatography with DCM:MeOH (10:1) to afford the intermediate (66) as a colorless oil (114 mg, 90% yield). ¹H NMR (CDCl₃, 400 MHz) δ (ppm) 7.65 (d, J=8.8 Hz, 2H), 7.42 (d, J=7.2 Hz, 1H), 7.19 (s, 1H), 7.08 (d, J=8.0 Hz, 2H), 6.81 (d, J=9.2 Hz, 1H), 6.59-6.54 (m, 4H), 3.99-3.95 (m, 2H), 3.92 (s, 2H), 3.48-3.40 (m, 4H), 2.86-2.82 (m, 2H), 2.64 (q, J=6.8 Hz, 2H), 2.55-2.51 (m, 2H), 1.59-1.52 (m, 2H), 1.44 (s, 9H), 1.31-1.25 (m, 2H), 1.01 (t, J=6.8 Hz, 3H); ¹³C NMR (CDCl₃, 100 MHz) δ (ppm) 194.16, 170.28, 162.73, 157.39, 154.83, 143.60, 140.09, 133.37, 132.59, 130.47, 129.90, 125.01, 124.06, 116.02, 115.57, 114.20, 107.67, 82.05, 71.67, 68.75, 53.54, 53.32, 51.72, 47.86, 29.78, 29.38, 28.18, 24.01, 10.64; UPLC-MS (ESI⁺) calc. for C₃₆H₄₄NO₇S [M+23]⁺: 634.28, found 634.18.

Trifluoroacetic acid (5.0 mL) was added to a solution of intermediate 66 (114 mg, 0.18 mmol) in DCM (10 mL) at 0° C. The solution was stirred at rt for 6 h. After concentration, the residue was purified by reversed-phase preparative HPLC to afford the title compound (67) as a slightly yellow solid (81 mg, 78% yield). UPLC-MS (ESI⁺) calc. for C₃₂H₃₆NO₇S [M+23]⁺: 578.22, found 578.06.

HATU (53 mg, 0.14 mmol, 1.0 eq) was added to a solution of intermediate 67 (81 mg, 0.14 mmol, 1.0 eq), compound 58 (67 mg, 0.15 mmol, 1.1 eq), and DIPEA (0.12 mL, 0.70 mmol, 5.0 eq) in DMF (2 mL). The mixture was stirred at rt for 1 h then was purified by reversed-phase preparative HPLC to afford the title compound 32 (ERD-308) as a yellow solid (56 mg, 40% yield). ¹H NMR (CD₃OD, 400 MHz) δ (ppm) 8.86 (s, 1H), 7.74 (d, J=9.2 Hz, 2H), 7.43-7.35 (m, 5H), 7.26 (d, J=2.0 Hz, 1H), 7.18 (d, J=8.4 Hz, 2H), 6.91-6.85 (m, 3H), 6.62 (d, J=8.4 Hz, 2H), 4.98-4.95 (m, 1H), 4.89 (s, 2H), 4.69-4.64 (m, 1H), 4.59-4.53 (m, 1H), 4.45-4.41 (m, 1H), 4.31 (t, J=4.8 Hz, 2H), 4.02-3.92 (m, 2H), 3.84 (d, J=11.2 Hz, 1H), 3.74 (dd, J=10.8 Hz, J=3.6 Hz, 1H), 3.56 (t, J=6.8 Hz, 2H), 3.45 (t, J=4.8 Hz, 2H), 3.11-3.07 (m, 2H), 2.47 (s, 3H), 2.22-2.19 (m, 1H), 1.98-1.92 (m, 1H), 1.76-1.66 (m, 4H), 1.57-1.46 (m, 5H), 1.02 (s, 9H); ¹³C NMR (CD₃OD, 100 MHz) δ (ppm) 195.36, 173.13, 173.05, 171.91, 171.80, 163.11, 159.29, 156.87, 152.90, 149.01, 145.60, 144.31, 141.47, 134.22, 133.55, 132.72, 131.45, 131.02, 130.49, 127.61, 127.38, 126.00, 124.72, 116.50, 116.13, 115.45, 107.93, 72.40, 70.90, 70.73, 69.08, 63.61, 60.68, 58.14, 54.33, 52.59, 50.26, 38.90, 37.80, 37.13, 29.90, 26.93, 24.56, 24.40, 22.43, 15.79, 9.18; UPLC-MS (ESI⁺) calc. for C₅₅H₆₆N₅O₉S₂ [M+1]⁺: 1004.43, found 1004.11; Purity 97.4% (HPLC).

Route B: exemplified by compound 15 (ERD-148).

### (2S,4R)-1-((S)-2-(8-(Ethyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (15, ERD-148)

Trifluoroacetic anhydride (3.80 mL, 27.34 mmol, 2.0 eq) was added at 0° C. to a solution of commercial 8-bromooctanoic acid (64, 3.05 g, 13.67 mmol, 1.0 eq) in 50 mL of DCM. The solution was stirred at rt for 2 h. Then tert-butanol (3.92 mL, 41.01 mmol, 3.0 eq) was added and the solution was stirred at rt for 12 h. Saturated aqueous NaHCO₃ was then added and the organic layer was separated and dried over anhydrous Na₂SO₄. After filtration and concentration, the residue was purified by silica gel flash column chromatography with hexane:EtOAc (20:1-5:1) to afford tert-butyl 8-bromooctanoate (65) as a colorless oil (2.48 g, 65% yield). ¹H NMR (CDCl₃, 400 MHz) δ (ppm) 3.34 (t, J=6.8 Hz, 2H), 2.14 (t, J=7.6 Hz, 2H), 1.83-1.75 (m, 2H), 1.54-1.49 (m, 2H), 1.40-1.36 (m, 11H), 1.29-1.25 (m, 4H); ¹³C NMR (CDCl₃, 100 MHz) δ (ppm) 173.08, 79.88, 35.47, 33.81, 32.73, 28.86, 28.44, 28.12, 27.99, 24.95;

Compound 15 (ERD-148) was prepared using a procedure similar to that used for compound 32 with intermediate 65 instead of compound 63 as the starting material. ¹H NMR (CD₃OD, 400 MHz) δ (ppm) 9.09 (s, 1H), 7.75 (d, J=8.8 Hz, 2H), 7.47-7.41 (m, 5H), 7.27 (d, J=2.4 Hz, 1H), 7.19-7.15 (m, 2H), 6.92 (d, J=8.8 Hz, 2H), 6.87 (dd, J=8.8 Hz, J=2.4 Hz, 1H), 6.63-6.59 (m, 2H), 5.02-4.90 (m, 1H), 4.64-4.54 (m, 2H), 4.43-4.41 (m, 1H), 4.35 (t, J=4.4 Hz, 2H), 3.88 (d, J=11.2 Hz, 1H), 3.74 (dd, J=11.2 Hz, J=4.0 Hz, 1H), 3.60 (t, J=4.8 Hz, 2H), 3.31-3.17 (m, 4H), 2.50 (s, 3H), 2.32-2.17 (m, 3H), 1.98-1.91 (m, 1H), 1.75-1.65 (m, 2H), 1.65-1.55 (m, 2H), 1.50 (d, J=6.8 Hz, 3H), 1.43-1.29 (m, 9H), 1.03 (s, 9H); ¹³C NMR (CD₃OD, 100 MHz) δ (ppm) 193.96, 174.53, 171.84, 170.93, 161.69, 157.88, 155.47, 152.13, 144.63, 142.99, 140.09, 132.82, 132.14, 131.39, 130.07, 129.62, 129.12, 126.33, 124.64, 123.31, 115.08, 114.74, 114.01, 106.51, 69.57, 62.17, 59.21, 57.61, 56.61, 53.15, 51.19, 48.85, 48.76, 37.41, 35.10, 28.54, 28.41, 25.93, 25.65, 25.33, 23.31, 20.96, 13.89, 7.67; UPLC-MS (ESI⁺) calc. for C₅₆H₆₈N₅O₈S₂ [M+1]⁺: 1002.45, found 1002.51; Purity 97.5% (HPLC).

### (2S,4R)-1-((S)-17-(tert-Butyl)-3-ethyl-1-(4-(6-hydroxy-2-(4-hydroxyphenyl)benzo[b]thiophene-3-carbonyl)phenoxy)-15-oxo-6,9,12-trioxa-3,16-diazaoctadecan-18-oyl)-4-hydroxy-N—((S)-1-(4-(4-methylthiazol-5-yl)phenyl)ethyl)pyrrolidine-2-carboxamide (14)

This compound was prepared using a procedure similar to that used for compound 32. ¹H NMR (CD₃OD, 400 MHz) δ (ppm) 8.87 (s, 1H), 7.72 (d, J=8.8 Hz, 2H), 7.44-7.39 (m, 5H), 7.26 (d, J=2.0 Hz, 1H), 7.19 (d, J=8.4 Hz, 2H), 6.89-6.85 (m, 3H), 6.63 (d, J=8.4 Hz, 2H), 5.02-4.96 (m, 1H), 4.64-4.54 (m, 2H), 4.43-4.41 (m, 1H), 4.20 (t, J=5.2 Hz, 2H), 3.85 (d, J=11.2 Hz, 1H), 3.75-3.64 (m, 5H), 3.60-3.55 (m, 8H), 3.20-2.93 (m, 6H), 2.54-2.46 (m, 5H), 2.20-2.17 (m, 1H), 1.98-1.92 (m, 1H), 1.49 (d, J=7.2 Hz, 3H), 1.67 (t, J=7.2 Hz, 3H), 1.02 (s, 9H); UPLC-MS (ESI⁺) calc. for C₅₇H₇₀N₅O₁₁S₂ [M+1]⁺: 1064.45, found 1064.74; Purity 96.4% (HPLC).

### (2S,4R)-1-((S)-2-(4-(Ethyl(2-(4-(6-hydroxy-2-(4-hydroxyphenyl)benzo[b]thiophene-3-carbonyl)phenoxy)ethyl)amino)butanamido)-3,3-dimethylbutanoyl)-4-hydroxy-N—((S)-1-(4-(4-methylthiazol-5-yl)phenyl)ethyl)pyrrolidine-2-carboxamide (16)

This compound was prepared using a procedure similar to that used for compound 15. ¹H NMR (CD₃OD, 400 MHz) δ (ppm) 8.87 (s, 1H), 7.73 (d, J=8.8 Hz, 2H), 7.45-7.39 (m, 5H), 7.26 (d, J=2.0 Hz, 1H), 7.18 (d, J=8.8 Hz, 1H), 6.90 (d, J=8.8 Hz, 2H), 6.86 (dd, J=8.8 Hz, J=2.4 Hz, 1H), 6.62 (d, J=8.8 Hz, 2H), 5.00-4.95 (m, 1H), 4.56-4.50 (m, 2H), 4.38-4.36 (m, 1H), 4.26 (t, J=4.8 Hz, 2H), 3.83 (d, J=11.2 Hz, 1H), 3.66 (dd, J=10.8 Hz, J=4.0 Hz, 1H), 3.06-2.99 (m, 4H), 2.47 (s, 3H), 2.43 (t, J=6.4 Hz, 2H), 2.20-2.15 (m, 1H), 1.97-1.89 (m, 3H), 1.49 (d, J=6.8 Hz, 3H), 1.23 (t, J=7.2 Hz, 3H), 1.00 (s, 9H); UPLC-MS (ESI⁺) calc. for C₅₂H₆₀N₅O₈S₂ [M+1]⁺: 946.39, found 946.41; Purity 97.4% (HPLC);

### (2S,4R)-1-((S)-2-(5-(Ethyl(2-(4-(6-hydroxy-2-(4-hydroxyphenyl)benzo[b]thiophene-3 carbonyl)phenoxy)ethyl)amino)pentanamido)-3,3-dimethylbutanoyl)-4-hydroxy-N—((S)-1-(4-(4-methylthiazol-5-yl)phenyl)ethyl)pyrrolidine-2-carboxamide (17)

This compound was prepared using a procedure similar to that used for compound 15. ¹H NMR (CD₃OD, 400 MHz) δ (ppm) 8.87 (s, 1H), 7.76-7.73 (m, 1H), 7.45-7.38 (m, 5H), 7.27 (d, J=2.0 Hz, 1H), 7.20-7.14 (m, 2H), 6.94-6.92 (m, 2H), 6.89-6.85 (m, 1H), 6.62-6.59 (m, 2H), 5.01-4.97 (m, 1H), 4.59-4.52 (m, 2H), 4.43-4.41 (m, 1H), 4.36 (t, J=4.8 Hz, 2H), 3.86 (d, J=11.2 Hz, 1H), 3.72 (dd, J=10.8 Hz, 0.7=4.0 Hz, 1H), 3.59 (t, J=4.8 Hz, 2H), 3.31-3.21 (m, 4H), 2.47 (s, 3H), 2.37 (t, J=6.8 Hz, 2H), 2.21-2.16 (m, 1H), 1.98-1.91 (m, 1H), 1.79-1.68 (m, 4H), 1.48 (d, J=7.2 Hz, 3H), 1.34 (t, J=7.2 Hz, 3H), 1.04 (s, 9H); UPLC-MS (ESI⁺) calc. for C₅₃H₆₂N₅O₈S₂ [M+1]⁺: 960.40, found 960.84; Purity 96.9% (HPLC).

### (2S,4R)-1-((S)-2-(6-(Ethyl(2-(4-(6-hydroxy-2-(4-hydroxyphenyl)benzo[b]thiophene-3-carbonyl)phenoxy)ethyl)amino)hexanamido)-3,3-dimethylbutanoyl)-4-hydroxy-N—((S)-1-(4-(4-methylthiazol-5-yl)phenyl)ethyl)pyrrolidine-2-carboxamide (18)

This compound was prepared using a procedure similar to that used for compound 15. ¹H NMR (CD₃OD, 400 MHz) δ (ppm) 8.87 (s, 1H), 7.73 (d, J=9.2 Hz, 2H), 7.44-7.38 (m, 5H), 7.26 (d, J=2.4 Hz, 1H), 7.16 (d, J=8.8 Hz, 2H), 6.90 (d, J=9.2 Hz, 2H), 6.87 (dd, J=8.8 Hz, J=2.0 Hz, 1H), 6.61 (d, J=8.8 Hz, 2H), 5.03-4.96 (m, 1H), 4.61-4.54 (m, 2H), 4.41-4.39 (m, 1H), 4.35 (t, J=4.8 Hz, 2H), 3.87-3.84 (m, 1H), 3.73-3.71 (m, 1H), 3.58 (t, J=4.4 Hz, 2H), 3.31-3.17 (m, 4H), 2.47 (s, 3H), 2.37-2.26 (m, 2H), 2.21-2.16 (m, 1H), 1.97-1.91 (m, 1H), 1.77-1.62 (m, 4H), 1.49 (d, J=7.2 Hz, 3H), 1.45-1.30 (m, 5H), 1.02 (s, 9H); UPLC-MS (ESI⁺) calc. for C₅₄H₆₄N₅O₈S₂ [M+1]⁺: 974.42, found 974.63; Purity 99.6% (HPLC).

### (2S,4R)-1-((S)-2-(7-(Ethyl(2-(4-(6-hydroxy-2-(4-hydroxyphenyl)benzo[b]thiophene-3-carbonyl)phenoxy)ethyl)amino)heptanamido)-3,3-dimethylbutanoyl)-4-hydroxy-N—((S)-1-(4-(4-methylthiazol-5-yl)phenyl)ethyl)pyrrolidine-2-carboxamide (19)

This compound was prepared using a procedure similar to that used for compound 15. ¹H NMR (CD₃OD, 400 MHz) δ (ppm) 8.86 (s, 1H), 7.71 (d, J=8.8 Hz, 2H), 7.44-7.38 (m, 5H), 7.26 (d, J=2.0 Hz, 1H), 7.17 (d, J=8.8 Hz, 2H), 6.89-6.80 (m, 3H), 6.62 (d, J=8.8 Hz, 2H), 5.02-4.97 (m, 1H), 4.63-4.55 (m, 2H), 4.43-4.41 (m, 1H), 4.24 (t, J=4.8 Hz, 2H), 3.87 (d, J=10.8 Hz, 1H), 3.74 (dd, J=10.8 Hz, J=4.0 Hz, 1H), 3.32-3.30 (m, 2H), 3.04-2.90 (m, 4H), 2.47 (s, 3H), 2.34-2.16 (m, 3H), 1.98-1.91 (m, 1H), 1.63-1.60 (m, 4H), 1.49 (d, J=7.2 Hz, 3H), 1.37-1.35 (m, 4H), 1.25-1.18 (m, 3H), 1.01 (s, 9H); UPLC-MS (ESI⁺) calc. for C₅₅H₆₆N₅O₈S₂ [M+1]⁺: 988.44, found 988.60; Purity 96.2% (HPLC).

### (2S,4R)-1-((S)-2-(9-(Ethyl(2-(4-(6-hydroxy-2-(4-hydroxyphenyl)benzo[b]thiophene-3-carbonyl)phenoxy)ethyl)amino)nonanamido)-3,3-dimethylbutanoyl)-4-hydroxy-N—((S)-1-(4-(4-methylthiazol-5-yl)phenyl)ethyl)pyrrolidine-2-carboxamide (20)

This compound was prepared using a procedure similar to that for compound 15. ¹H NMR (CD₃OD, 400 MHz) δ (ppm) 8.87 (s, 1H), 7.75-7.70 (m, 2H), 7.44-7.39 (m, 5H), 7.26 (d, J=2.4 Hz, 1H), 7.18-7.15 (m, 2H), 6.90 (d, J=8.8 Hz, 2H), 6.87 (dd, J=8.8 Hz, J=2.4 Hz, 1H), 6.61 (d, J=8.8 Hz, 2H), 5.04-4.88 (m, 1H), 4.64-4.55 (m, 2H), 4.45-4.40 (m, 1H), 4.35 (t, J=4.8 Hz, 2H), 3.89-3.86 (m, 1H), 3.74 (dd, J=10.8 Hz, J=4.0 Hz, 1H), 3.58 (t, J=4.8 Hz, 2H), 3.31-3.28 (m, 2H), 3.21-3.15 (m, 2H), 2.47 (s, 3H), 2.31-2.17 (m, 3H), 1.98-1.92 (m, 1H), 1.75-1.55 (m, 4H), 1.50 (d, J=7.2 Hz, 3H), 1.36-1.31 (m, 11H), 1.03 (s, 9H); UPLC-MS (ESI⁺) calc. for C₅₇H₇₀N₅O₈S₂ [M+1]⁺: 1016.47, found 1016.53; Purity 95.7% (HPLC).

### (2S,4R)-1-((S)-2-(10-(Ethyl(2-(4-(6-hydroxy-2-(4-hydroxyphenyl)benzo[b]thiophene-3-carbonyl)phenoxy)ethyl)amino)decanamido)-3,3-dimethylbutanoyl)-4-hydroxy-N—((S)-1-(4-(4-methylthiazol-5-yl)phenyl)ethyl)pyrrolidine-2-carboxamide (21)

This compound was prepared using a procedure similar to that used for compound 15. ¹H NMR (CD₃OD, 400 MHz) δ (ppm) 8.86 (s, 1H), 7.72 (d, J=8.8 Hz, 2H), 7.43-7.36 (m, 5H), 7.26 (d, J=2.0 Hz, 1H), 7.17 (d, 8.8 Hz, 2H), 6.89-6.84 (m, 3H), 6.61 (d, J=8.8 Hz, 2H), 5.02-4.97 (m, 1H), 4.64-4.55 (m, 2H), 4.43-4.41 (m, 1H), 4.27 (t, J=4.8 Hz, 2H), 3.88 (d, J=11.2 Hz, 1H), 3.74 (dd, J=11.2 Hz, J=4.0 Hz, 1H), 3.39 (t, J=4.4 Hz, 2H), 3.15-3.09 (m, 2H), 3.03-2.98 (m, 2H), 2.46 (s, 3H), 2.33-2.17 (m, 3H), 1.98-1.92 (m, 1H), 1.69-1.50 (m, 4H), 1.49 (d, J=7.2 Hz, 3H), 1.33-1.23 (m, 13H), 1.03 (s, 9H); UPLC-MS (ESI⁺) calc. for C₅₈H₇₂N₅O₈S₂ [M+1]⁺: 1030.48, found 1030.46; Purity 96.4% (HPLC).

### (2S,4R)-4-Hydroxy-1-((S)-2-(8-((2-(4-(6-hydroxy-2-(4-hydroxyphenyl)benzo[b]thiophene-3-carbonyl)phenoxy)ethyl)(methyl)amino)octanamido)-3,3-dimethylbutanoyl)-N—((S)-1-(4-(4-methylthiazol-5-yl)phenyl)ethyl)pyrrolidine-2-carboxamide (22)

This compound was prepared using a procedure similar to that used for compound 15. ¹H NMR (CD₃OD, 400 MHz) δ (ppm) 8.95 (s, 1H), 7.74 (d, J=9.2 Hz, 2H), 7.45-7.42 (m, 5H), 7.27 (d, J=2.0 Hz, 1H), 7.17 (d, J=8.4 Hz, 2H), 6.92 (d, J=8.8 Hz, 2H), 6.87 (dd, J=8.8 Hz, J=2.4 Hz, 1H), 6.61 (d, J=8.8 Hz, 2H), 5.02-4.97 (m, 1H), 4.64-4.54 (m, 2H), 4.42-4.37 (m, 3H), 3.87 (d, J=11.2 Hz, 1H), 3.74 (dd, J=11.2 Hz, J=4.4 Hz, 1H), 3.66-3.48 (m, 2H), 3.23-3.13 (m, 2H), 2.93 (s, 3H), 2.48 (s, 3H), 2.34-2.17 (m, 3H), 1.98-1.91 (m, 1H), 1.80-1.70 (m, 2H), 1.63-1.55 (m, 2H), 1.50 (d, J=7.2 Hz, 3H), 1.45-1.35 (m, 6H), 1.03 (s, 9H); UPLC-MS (ESI⁴) calc. for C₅₅H₆₆N₅O₈S₂ [M+1]⁺: 988.44, found 988.54; Purity 95.0% (HPLC).

### (2S,4R)-4-Hydroxy-1-((S)-2-(8-((2-(4-(6-hydroxy-2-(4-hydroxyphenyl)benzo[b]thiophene-3-carbonyl)phenoxy)ethyl)(isopropyl)amino)octanamido)-3,3-dimethylbutanoyl)-N—((S)-1-(4-(4-methylthiazol-5-yl)phenyl)ethyl)pyrrolidine-2-carboxamide (23)

This compound was prepared using a procedure similar to that used for compound 15. ¹H NMR (CD₃OD, 400 MHz) δ (ppm) 8.86 (s, 1H), 7.72 (d, J=8.8 Hz, 2H), 7.43-7.38 (m, 5H), 7.26 (d, J=2.0 Hz, 1H), 7.17 (d, J=8.8 Hz, 2H), 6.88-6.84 (m, 3H), 6.62 (d, J=8.8 Hz, 2H), 5.02-4.97 (m, 1H), 4.63-4.55 (m, 2H), 4.43-4.41 (m, 1H), 4.16 (t, J=5.2 Hz, 2H), 3.87 (d, J=11.2 Hz, 1H), 3.74 (dd, J=10.8 Hz, J=4.0 Hz, 1H), 3.37-3.35 (m, 1H), 2.86-2.82 (m, 2H), 2.46 (s, 3H), 2.31-2.16 (m, 3H), 1.98-1.92 (m, 1H), 1.59-1.56 (m, 4H), 1.49 (d, J=6.8 Hz, 3H), 1.33-1.27 (m, 10H), 1.18 (d, J=6.8 Hz, 6H), 1.03 (s, 9H); UPLC-MS (ESI⁺) calc. for C₅₇H₇₀N₅O₈S₂ [M+1]⁺: 1016.47, found 1016.55; Purity 96.0% (HPLC).

### (2S,4R)-1-((S)-2-(8-(tert-Butyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (ERD-107-WMA, 24)

This compound was prepared using a procedure similar to that used for compound 15. ¹H NMR (CD₃OD, 400 MHz) δ (ppm) 8.97 (s, 1H), 7.76 (d, J=8.8 Hz, 2H), 7.47-7.40 (m, 5H), 7.26 (d, J=2.4 Hz, 1H), 7.18 (d, J=8.8 Hz, 2H), 6.92-6.86 (m, 3H), 6.62-6.60 (m, 2H), 5.03-4.97 (m, 1H), 4.62 (s, 1H), 4.59-4.55 (m, 1H), 4.45-4.41 (m, 1H), 4.32 (t, J=4.4 Hz, 2H), 3.91-3.83 (m, 2H), 3.74 (dd, J=11.2 Hz, J=4.0 Hz, 1H), 3.49-3.38 (m, 2H), 3.14-3.10 (m, 1H), 2.48 (s, 3H), 2.33-2.17 (m, 3H), 1.99-1.92 (m, 1H), 1.83-1.73 (m, 1H), 1.70-1.55 (m, 4H), 1.51-1.47 (m, 3H), 1.47-1.45 (m, 9H), 1.39-1.29 (m, 6H), 1.03 (s, 9H); ¹³C NMR (CD₃OD, 100 MHz) δ (ppm) 195.34, 175.92, 173.23, 172.33, 163.09, 159.30, 156.89, 153.17, 148.54, 145.80, 144.45, 141.51, 134.22, 133.59, 132.77, 131.50, 131.25, 130.98, 130.51, 127.67, 127.45, 126.04, 124.72, 116.47, 116.15, 115.30, 107.91, 70.97, 67.43, 65.32, 60.63, 59.02, 58.00, 53.38, 51.70, 50.16, 49.71, 38.82, 36.50, 29.99, 29.86, 27.63, 27.53, 27.06, 26.76, 24.94, 22.37, 15.57; UPLC-MS (ESE) calc. for C₅₈H₇₂N₅O₈S₂ [M+1]⁺: 1030.48, found 1030.52.

### (2S,4R)-1-((S)-2-(8-(Cyclopropyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (25)

This compound was prepared using a procedure similar to that used for compound 15. ¹H NMR (CD₃OD, 400 MHz) δ (ppm) 9.04 (s, 1H), 7.73 (d, J=9.2 Hz, 2H), 7.46-7.41 (m, 5H), 7.26 (d, J=2.0 Hz, 1H), 7.16 (d, J=8.8 Hz, 2H), 6.91-6.86 (m, 3H), 6.61 (d, J=8.8 Hz, 2H), 5.03-4.97 (m, 1H), 4.64-4.61 (m, 1H), 4.57 (t, J=8.4 Hz, 1H), 4.42-4.29 (m, 3H), 3.87 (d, J=11.2 Hz, 1H), 3.76-3.65 (m, 3H), 3.32-3.30 (m, 1H), 2.89-2.84 (m, 1H), 2.49 (s, 3H), 2.33-2.17 (m, 3H), 1.99-1.92 (m, 1H), 1.85-1.75 (m, 2H), 1.63-1.55 (m, 2H), 1.50 (d, J=7.2 Hz, 3H), 1.45-1.35 (m, 6H), 1.03 (s, 9H), 1.00-0.90 (m, 4H); ¹³C NMR (CD₃OD, 100 MHz) δ (ppm) 195.30, 175.92, 173.23, 172.33, 162.98, 159.26, 156.84, 153.43, 145.96, 144.57, 141.46, 134.21, 133.53, 132.77, 131.50, 131.02, 130.49, 127.70, 126.01, 124.75, 116.45, 116.14, 115.38, 107.92, 70.95, 63.22, 60.61, 59.01, 57.98, 57.85, 55.33, 50.14, 38.78, 38.54, 36.47, 29.94, 29.81, 27.42, 27.04, 26.72, 24.72, 22.34, 15.31; UPLC-MS (ESI⁺) calc. for C₅₇H₆₈N₅O₈S₂ [M+1]⁺: 1014.45, found 1014.61; Purity 96.1% (HPLC).

### (2S,4R)-1-((S)-2-(8-(Cyclobutyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (26)

This compound was prepared using a procedure similar to that used for compound 15. ¹H NMR (CD₃OD, 400 MHz) δ (ppm) 9.04 (s, 1H), 7.74 (d, J=8.8 Hz, 2H), 7.44-7.42 (m, 5H), 7.26 (d, J=2.4 Hz, 1H), 7.17 (d, J=8.8 Hz, 2H), 6.91-6.85 (m, 3H), 6.62 (d, J=8.8 Hz, 2H), 5.03-4.97 (m, 1H), 4.64-4.55 (m, 2H), 4.45-4.41 (m, 1H), 4.32-4.29 (m, 2H), 3.91-3.86 (m, 2H), 3.74 (dd, J=11.2 Hz, J=4.0 Hz, 1H), 3.49 (t, J=4.8 Hz, 2H), 3.09 (t, J=8.8 Hz, 2H), 2.48 (s, 3H), 2.35-2.15 (m, 7H), 1.99-1.92 (m, 1H), 1.87-1.55 (m, 6H), 1.50 (d, J=7.2 Hz, 3H), 1.40-1.30 (m, 6H), 1.03 (s, 9H); ¹³C NMR (CD₃OD, 100 MHz) δ (ppm) 195.32, 175.91, 173.22, 172.33, 163.01, 159.27, 156.85, 153.42, 145.95, 144.39, 141.46, 134.22, 133.54, 132.73, 131.45, 131.01, 130.49, 127.70, 125.99, 124.73, 116.49, 116.15, 115.37, 107.94, 70.95, 63.46, 60.60, 59.61, 59.01, 57.98, 52.11, 50.49, 50.14, 38.78, 36.49, 36.47, 29.94, 29.79, 27.40, 27.05, 26.71, 24.30, 22.35, 15.33, 14.18; UPLC-MS (ESI⁺) calc. for C₅₈H₇₀N₅O₈S₂ [M+1]⁺: 1028.47, found 1029.18; Purity 97.7% (HPLC).

### (2S,4R)-1-((S)-2-(8-(Cyclopentyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (27)

This compound was prepared using a procedure similar to that used for compound 15. ¹H NMR (CD₃OD, 400 MHz) δ (ppm) 8.93 (s, 1H), 7.75 (d, J=8.8 Hz, 2H), 7.45-7.41 (m, 5H), 7.26 (d, J=2.4 Hz, 1H), 7.18 (d, J=8.8 Hz, 2H), 6.92-6.85 (m, 3H), 6.61 (d, J=8.8 Hz, 2H), 5.02-4.98 (m, 1H), 4.64-4.55 (m, 2H), 4.43-4.41 (m, 1H), 4.37-4.33 (m, 2H), 3.89-3.73 (m, 3H), 3.65-3.55 (m, 2H), 3.22 (t, J=8.4 Hz, 2H), 2.48 (s, 3H), 2.30-2.15 (m, 5H), 2.03-1.94 (m, 1H), 1.84-1.57 (m, 10H), 1.50 (d, J=7.2 Hz, 3H), 1.45-1.30 (m, 6H), 1.03 (s, 9H); ¹³C NMR (CD₃OD, 100 MHz) δ (ppm) 195.34, 175.89, 173.21, 172.34, 163.06, 159.29, 156.87, 153.06, 148.67, 145.73, 144.37, 141.49, 134.22, 133.55, 132.78, 131.46, 131.32, 131.01, 130.49, 127.65, 127.42, 126.03, 124.71, 116.48, 116.14, 115.36, 107.92, 70.96, 67.31, 63.94, 60.62, 59.00, 57.99, 54.01, 52.09, 38.80, 36.50, 29.97, 29.84, 29.20, 29.14, 27.39, 27.05, 26.72, 24.85, 24.81, 24.66, 22.34, 15.61; UPLC-MS (ESI⁴) calc. for C₅₉H₇₂N₅O₈S₂ [M+1]⁺: 1042.48, found 1042.39; Purity >99.5% (HPLC).

### (2S,4R)-1-((S)-2-(8-(Cyclohexyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (28, ERD-045-WMA)

This compound was prepared using a procedure similar to that used for compound 15. ¹H NMR (CD₃OD, 400 MHz) δ (ppm) 8.87 (s, 1H), 7.75 (d, J=8.8 Hz, 2H), 7.45-7.39 (m, 5H), 7.26 (d, J=2.0 Hz, 1H), 7.18 (d, J=8.4 Hz, 2H), 6.91-6.84 (m, 3H), 6.62-6.60 (m, 2H), 5.02-4.97 (m, 1H), 4.64-4.54 (m, 2H), 4.45-4.41 (m, 1H), 4.32 (t, J=4.4 Hz, 2H), 3.89-3.85 (m, 1H), 3.74 (dd, J=11.2 Hz, J=4.0 Hz, 1H), 3.59-3.45 (m, 2H), 3.18-3.08 (m, 2H), 2.47 (s, 3H), 2.34-2.16 (m, 3H), 2.03-1.90 (m, 5H), 1.74-1.31 (m, 20H), 1.03 (s, 9H); ¹³C NMR (CD₃OD, 100 MHz) δ (ppm) 195.37, 175.94, 173.22, 172.33, 163.29, 159.29, 156.87, 152.87, 149.07, 145.61, 144.35, 141.49, 134.23, 133.56, 133.34, 131.53, 131.47, 131.03, 130.50, 127.62, 127.41, 126.04, 116.47, 116.13, 115.36, 107.91, 70.96, 60.62, 59.01, 58.00, 53.41, 51.06, 50.15, 49.28, 38.81, 37.63, 36.50, 30.00, 29.89, 27.58, 27.05, 26.20, 26.19, 26.18, 26.17, 26.15, 26.14, 22.36, 15.79; UPLC-MS (ESE) calc. for C₆₀H₇₄N₅O₈S₂ [M+1]⁺: 1056.50, found 1056.54.

### (2S,4R)-1-((S)-2-(8-((Cyclobutylmethyl)(2-(4-(6-hydroxy-2-(4-hydroxyphenyl)benzo-[b]thiophene-3-carbonyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (29)

This compound was prepared using a procedure similar to that used for compound 15. ¹H NMR (CD₃OD, 400 MHz) δ (ppm) 8.98 (s, 1H), 7.73 (d, J=8.8 Hz, 2H), 7.44-7.36 (m, 5H), 7.26 (d, J=2.0 Hz, 1H), 7.17 (d, J=8.4 Hz, 2H), 6.90-6.85 (m, 3H), 6.61 (d, J=8.4 Hz, 2H), 5.02-4.97 (m, 1H), 4.64-4.55 (m, 2H), 4.45-4.41 (m, 1H), 4.33 (t, 0.7=4.4 Hz, 2H), 3.87 (d, J=11.2 Hz, 1H), 3.74 (dd, J=11.2 Hz, 0.7=4.0 Hz, 1H), 3.54-3.52 (m, 2H), 3.28-3.25 (m, 2H), 3.16-3.11 (m, 2H), 2.80-2.73 (m, 1H), 2.48 (s, 3H), 2.32-2.14 (m, 5H), 2.04-1.84 (m, 5H), 1.75-1.65 (m, 2H), 1.65-1.55 (m, 2H), 1.49 (d, J=7.2 Hz, 3H), 1.42-1.30 (m, 6H), 1.03 (s, 9H); ¹³C NMR (CD₃OD, 100 MHz) δ (ppm) 195.33, 175.89, 173.20, 172.32, 163.05, 159.27, 156.84, 153.25, 148.24, 145.84, 144.30, 141.45, 134.22, 133.52, 132.72, 131.43, 131.07, 131.01, 130.48, 127.67, 125.97, 124.72, 116.50, 116.14, 115.39, 107.95, 70.94, 63.50, 60.60, 60.11, 59.00, 57.98, 55.21, 51.06, 50.13, 38.78, 36.48, 31.78, 29.94, 29.81, 28.20, 28.09, 27.35, 27.05, 26.71, 24.52, 22.34, 19.42, 15.46; UPLC-MS (ESI⁺) calc. for C₅₉H₇₂N₅O₈S₂ [M+1]⁺: 1042.48, found 1042.82; Purity >99.5% (HPLC).

### (2S,4R)-1-((S)-14-(tert-Butyl)-3-ethyl-1-(4-(6-hydroxy-2-(4-hydroxyphenyl)benzo[b]-thiophene-3-carbonyl)phenoxy)-12-oxo-6,9-dioxa-3,13-diazapentadecan-15-oyl)-4-hydroxy-N—((S)-1-(4-(4-methylthiazol-5-yl)phenyl)ethyl)pyrrolidine-2-carboxamide (30)

This compound was prepared using a procedure similar to that used for compound 32. ¹H NMR (CD₃OD, 400 MHz) δ (ppm) 8.86 (s, 1H), 7.74 (d, J=8.8 Hz, 2H), 7.44-7.37 (m, 5H), 7.27 (d, J=2.4 Hz, 1H), 7.18 (d, J=8.8 Hz, 2H), 6.93 (d, J=8.8 Hz, 2H), 6.87 (dd, J=8.8 Hz, J=2.4 Hz, 1H), 6.62 (d, J=8.4 Hz, 2H), 5.02-4.95 (m, 1H), 4.57-4.53 (m, 2H), 4.40-4.37 (m, 3H), 3.85-3.80 (m, 3H), 3.73-3.58 (m, 9H), 3.45 (t, J=8.8 Hz, 2H), 3.37 (q, J=7.2 Hz, 2H), 2.55-2.44 (m, 5H), 2.22-2.17 (m, 1H), 1.97-1.91 (m, 1H), 1.48 (d, J=7.2 Hz, 3H), 1.35 (t, J=7.2 Hz, 3H), 1.01 (s, 9H); UPLC-MS (ESI⁺) calc. for C₅₅H₆₆N₅O₁₀S₂ [M+1]⁺: 1020.43, found 1020.77; Purity 97.2% (HPLC).

### (2S,4R)-1-((S)-2-(tert-Butyl)-12-ethyl-14-(4-(6-hydroxy-2-(4-hydroxyphenyl)benzo[b]-thiophene-3-carbonyl)phenoxy)-4-oxo-6,9-dioxa-3,12-diazatetradecanoyl)-4-hydroxy-N—((S)-1-(4-(4-methylthiazol-5-yl)phenyl)ethyl)pyrrolidine-2-carboxamide (31)

This compound was prepared using a procedure similar to that used for compound 32. ¹H NMR (CD₃OD, 400 MHz) δ (ppm) 9.11 (s, 1H), 7.73 (d, J=8.4 Hz, 2H), 7.44-7.37 (m, 5H), 7.26 (d, J=2.0 Hz, 1H), 7.17 (d, J=8.8 Hz, 2H), 6.92-6.84 (m, 3H), 6.61 (d, J=8.4 Hz, 2H), 4.97-4.91 (m, 1H), 4.71-4.68 (m, 1H), 4.57-4.54 (m, 1H), 4.41-4.38 (m, 3H), 4.02-3.40 (m, 16H), 2.48 (s, 3H), 2.36-2.20 (m, 1H), 1.95-1.89 (m, 1H), 1.45 (d, J=7.2 Hz, 2H), 1.37 (t, J=7.6 Hz, 3H), 1.01 (s, 9H); UPLC-MS (ESI⁺) calc. for C₅₄H₆₄N₅O₁₀S₂ [M+1]⁺: 1006.41, found 1006.66; Purity 95.1% (HPLC).

### (2S,4R)-1-((S)-2-(4-(4-(2-(Ethyl(2-(4-(6-hydroxy-2-(4-hydroxyphenyl)benzo[b]thiophene-3-carbonyl)phenoxy)ethyl)amino)ethyl)piperidin-1-yl)butanamido)-3,3-dimethylbutanoyl)-4-hydroxy-N—((S)-1-(4-(4-methylthiazol-5-yl)phenyl)ethyl)pyrrolidine-2-carboxamide (33)

This compound was prepared using a procedure similar to that used for compound 32. ¹H NMR (CD₃OD, 400 MHz) δ (ppm) 8.95 (s, 1H), 7.74 (d, J=8.8 Hz, 2H), 7.45-7.40 (m, 5H), 7.27 (d, J=2.4 Hz, 1H), 7.17 (d, J=8.4 Hz, 2H), 6.93 (d, J=9.2 Hz, 2H), 6.87 (dd, J=8.8 Hz, J=2.4 Hz, 1H), 6.60 (d, J=8.4 Hz, 2H), 5.03-4.90 (m, 1H), 4.59-4.53 (m, 2H), 4.43-4.41 (m, 1H), 4.37 (t, J=4.8 Hz, 2H), 3.90 (d, J=10.8 Hz, 1H), 3.74 (dd, J=10.8 Hz, J=4.0 Hz, 1H), 3.61-3.56 (m, 4H), 3.35-3.27 (m, 4H), 3.10 (t, J=6.8 Hz, 2H), 2.94-2.86 (m, 2H), 2.51 (t, J=6.4 Hz, 2H), 2.48 (s, 3H), 2.22-2.17 (m, 1H), 2.01-1.95 (m, 5H), 1.78-1.68 (m, 3H), 1.59-1.57 (m, 2H), 1.50 (d, J=7.2 Hz, 3H), 1.34 (t, J=7.6 Hz, 3H), 1.06 (s, 9H); UPLC-MS (ESI⁺) calc. for C₅₉H₇₃N₆O₈S₂ [M+1]⁺: 1057.49, found 1057.90; Purity 99.1% (HPLC).

### (2S,4R)-1-((S)-2-(4-(4-(2-(Ethyl(2-(4-(6-hydroxy-2-(4-hydroxyphenyl)benzo[b]thiophene-3-carbonyl)phenoxy)ethyl)amino)ethyl)piperazin-1-yl)butanamido)-3,3-dimethylbutanoyl)-4-hydroxy-N—((S)-1-(4-(4-methylthiazol-5-yl)phenyl)ethyl)pyrrolidine-2-carboxamide (34)

This compound was prepared using a procedure similar to that used for compound 32. ¹H NMR (CD₃OD, 400 MHz) δ (ppm) 8.94 (s, 1H), 7.75 (d, J=8.8 Hz, 2H), 7.45-7.40 (m, 5H), 7.27 (d, J=2.4 Hz, 1H), 7.18 (d, J=8.4 Hz, 2H), 6.93 (d, J=9.2 Hz, 2H), 6.87 (dd, J=8.8 Hz, J=2.4 Hz, 1H), 6.61 (d, J=8.4 Hz, 2H), 5.03-4.99 (m, 1H), 4.58-4.54 (m, 2H), 4.44-4.39 (m, 3H), 3.90 (d, J=10.8 Hz, 1H), 3.76-3.70 (m, 3H), 3.51-3.48 (m, 2H), 3.42-3.37 (m, 2H), 3.14-3.12 (m, 2H), 2.85 (t, J=6.4 Hz, 2H), 2.53 (t, J=6.4 Hz, 2H), 2.48 (s, 3H), 2.23-2.18 (m, 1H), 2.01-1.92 (m, 3H), 1.50 (d, J=6.8 Hz, 3H), 1.36 (t, J=7.2 Hz, 3H), 1.06 (s, 9H); UPLC-MS (ESI⁺) calc. for C₅₈H₇₂N₇O₈S₂ [M+1]⁺: 1058.49, found 1058.72; Purity 99.3% (HPLC).

### (2S,4R)-1-((S)-2-(3-(4-(5-(Ethyl(2-(4-(6-hydroxy-2-(4-hydroxyphenyl)benzo[b]thiophene-3-carbonyl)phenoxy)ethyl)amino)pent-1-yn-1-yl)-1H-pyrazol-1-yl)propanamido)-3,3-dimethylbutanoyl)-4-hydroxy-N—((S)-1-(4-(4-methylthiazol-5-yl)phenyl)ethyl)pyrrolidine-2-carboxamide (35)

This compound was prepared using a procedure similar to that used for compound 32. ¹H NMR (CD₃OD, 400 MHz) δ (ppm) 9.13 (s, 1H), 7.69 (d, J=8.8 Hz, 2H), 7.62 (s, 1H), 7.46-7.41 (m, 6H), 7.27 (d, J=2.0 Hz, 1H), 7.17 (d, J=8.4 Hz, 2H), 6.90-6.86 (m, 3H), 6.61 (d, J=8.8 Hz, 2H), 5.01-4.96 (m, 1H), 4.59-4.55 (m, 2H), 4.42-4.26 (m, 5H), 3.86 (d, J=10.8 Hz, 1H), 3.72 (dd, J=10.8 Hz, J=4.0 Hz, 1H), 3.62-3.57 (m, 2H), 3.41-3.30 (m, 4H), 2.92-2.85 (m, 1H), 2.75-2.70 (m, 1H), 2.54 (t, J=6.8 Hz, 2H), 2.50 (s, 3H), 2.22-2.17 (m, 1H), 2.01-1.91 (m, 3H), 1.50 (d, J=6.8 Hz, 3H), 1.36 (t, J=7.2 Hz, 3H), 0.95 (s, 9H); ¹³C NMR (CD₃OD, 100 MHz) δ (ppm) 195.31, 173.25, 172.24, 172.10, 163.01, 159.26, 156.86, 153.68, 146.13, 144.43, 142.84, 141.47, 134.21, 133.48, 132.76, 131.49, 131.00, 130.51, 127.74, 126.02, 124.77, 116.48, 116.15, 115.42, 107.92, 104.32, 88.60, 74.07, 70.96, 63.58, 60.60, 59.18, 57.88, 53.41, 52.83, 50.53, 50.45, 38.77, 36.74, 36.42, 26.98, 23.92, 22.36, 17.27, 15.15, 9.16; UPLC-MS (ESI⁺) calc. for C₅₉H₆₆N₇O₈S₂ [M+1]⁺: 1064.44, found 1064.89; Purity 95.1% (HPLC).

### (2S,4R)-1-((S)-2-(2-(4-(4-(Ethyl(2-(4-(6-hydroxy-2-(4-hydroxyphenyl)benzo[b]thiophene-3-carbonyl)phenoxy)ethyl)amino)but-1-yn-1-yl)-1H-pyrazol-1-yl)acetamido)-3,3-dimethyl-butanoyl)-4-hydroxy-N—((S)-1-(4-(4-methylthiazol-5-yl)phenyl)ethyl)pyrrolidine-2-carboxamide (36)

This compound was prepared using a procedure similar to that used for compound 32. ¹H NMR (CD₃OD, 400 MHz) δ (ppm) 8.87 (s, 1H), 7.74-7.70 (m, 3H), 7.52 (s, 1H), 7.44-7.41 (m, 5H), 7.26 (d, J=2.0 Hz, 1H), 7.18 (d, J=8.4 Hz, 2H), 6.88-6.85 (m, 3H), 6.63 (d, J=8.8 Hz, 2H), 5.01-4.98 (m, 1H), 4.62-4.52 (m, 2H), 4.42-4.39 (m, 3H), 3.82 (d, J=11.2 Hz, 1H), 3.76-3.64 (m, 3H), 3.50-3.39 (m, 6H), 2.96 (t, J=7.2 Hz, 2H), 2.47 (s, 3H), 2.26-2.15 (m, 1H), 1.96-1.90 (m, 1H), 1.49 (d, J=7.2 Hz, 3H), 1.38 (t, J=7.2 Hz, 3H), 1.02 (s, 9H); UPLC-MS (ESI⁺) calc. for C₅₇H₆₂N₇O₈S₂ [M+1]⁺: 1036.41, found 1035.92; Purity 98.8% (HPLC).

### (2S,4R)-1-((S)-2-(2-(4-(4-(Ethyl(2-(4-(6-hydroxy-2-(4-hydroxyphenyl)benzo[b]thiophene-3-carbonyl)phenoxy)ethyl)amino)butyl)-1H-pyrazol-1-yl)acetamido)-3,3-dimethylbutanoyl)-4-hydroxy-N—((S)-1-(4-(4-methylthiazol-5-yl)phenyl)ethyl)pyrrolidine-2-carboxamide (37)

This compound was prepared using a procedure similar to that used for compound 32. ¹H NMR (CD₃OD, 400 MHz) δ (ppm) 9.23 (s, 1H), 7.70 (d, J=8.8 Hz, 2H), 7.49 (s, 1H), 7.44-7.37 (m, 6H), 7.25 (d, J=2.4 Hz, 1H), 7.14 (d, J=8.8 Hz, 2H), 6.87-6.83 (m, 3H), 6.59 (d, J=8.4 Hz, 2H), 4.99-4.83 (m, 3H), 4.61-4.52 (m, 2H), 4.40-4.37 (m, 1H), 4.30 (t, J=4.4 Hz, 2H), 3.81 (d, J=11.2 Hz, 1H), 3.69 (dd, J=11.2 Hz, J=4.0 Hz, 1H), 3.54 (d, J=4.0 Hz, 2H), 3.28-3.16 (m, 4H), 2.54-2.46 (m, 5H), 2.22-2.17 (m, 1H), 1.96-1.89 (m, 1H), 1.73-1.60 (m, 4H), 1.47 (d, J=7.2 Hz, 3H), 1.30 (t, J=7.2 Hz, 3H), 1.00 (s, 9H); ¹³C NMR (CD₃OD, 100 MHz) δ (ppm) 195.28, 173.08, 171.67, 169.31, 163.05, 159.21, 156.78, 153.34, 147.93, 145.88, 144.26, 141.40, 140.79, 134.19, 133.92, 133.49, 132.59, 131.61, 131.41, 130.96, 130.89, 130.45, 127.65, 125.90, 124.74, 122.64, 116.52, 116.16, 115.42, 108.00, 70.90, 63.51, 60.56, 59.13, 57.96, 54.70, 54.16, 52.44, 50.14, 38.80, 36.70, 28.55, 26.93, 26.82, 24.24, 24.05, 22.38, 15.39, 9.07; UPLC-MS (ESI⁺) calc. for C₅₇H₆₆N₇O₈S₂ [M+1]⁺: 1040.44, found 1040.17; Purity 98.7% (HPLC).

### (2S,4R)-1-((S)-2-(8-(Ethyl(2-(4-((Z)-1-(4-hydroxyphenyl)-2-phenylbut-1-en-1-yl)-phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (38)

This compound was prepared using a procedure similar to that used for compound 15. ¹H NMR (CD₃OD, 400 MHz) δ (ppm) 8.98 (s, 1H), 7.47-7.36 (m, 4H), 7.13-7.01 (m, 7H), 6.84-6.75 (m, 3H), 6.66-6.60 (m, 2H), 5.03-4.98 (m, 1H), 4.64-4.62 (m, 1H), 4.56 (t, J=8.4 Hz, 1H), 4.45-4.41 (m, 1H), 4.22-4.18 (m, 2H), 3.89-3.86 (m, 1H), 3.74 (dd, J=11.2 Hz, J=4.0 Hz, 1H), 3.55-3.52 (m, 2H), 3.21-3.15 (m, 2H), 2.52-2.45 (m, 5H), 2.32-2.17 (m, 3H), 1.99-1.92 (m, 1H), 1.78-1.56 (m, 5H), 1.52-1.49 (m, 3H), 1.45-1.30 (m, 9H), 1.04 (s, 9H), 0.91 (t, J=7.2 Hz, 3H); ¹³C NMR (CD₃OD, 100 MHz) δ (ppm) 175.90, 173.22, 172.31, 157.41, 156.91, 153.20, 145.85, 144.03, 142.58, 139.49, 138.76, 136.07, 133.23, 131.55, 130.91, 130.51, 128.88, 127.67, 127.04, 115.92, 114.46, 70.96, 63.11, 60.60, 58.99, 58.00, 54.48, 52.76, 50.15, 38.82, 37.64, 36.51, 33.74, 29.95, 29.93, 29.86, 29.81, 27.36, 27.05, 26.73, 24.71, 22.37, 15.54, 13.86, 9.04; UPLC-MS (ESI⁺) calc. for C₅₇H₇₄N₅O₆S [M+1]⁺: 956.54, found 956.51.

### (2S,4R)-1-((S)-2-(8-((2-(4-(1,2-Diphenylbut-1-en-1-yl)phenoxy)ethyl)(ethyl)amino)-octanamido)-3,3-dimethylbutanoyl)-4-hydroxy-N—((S)-1-(4-(4-methylthiazol-5-yl)phenyl)ethyl)pyrrolidine-2-carboxamide (39)

This compound was prepared using a procedure similar to that used for compound 15. ¹H NMR (CD₃OD, 400 MHz) δ (ppm) 9.08 (s, 1H), 7.47-7.42 (m, 4H), 7.36-7.32 (m, 1H), 7.28-7.22 (m, 0.5H), 7.21-7.07 (m, 7H), 7.03-6.95 (m, 2.5H), 6.86-6.82 (m, 2H), 6.66-6.64 (m, 1H), 5.03-4.98 (m, 1H), 4.64-4.62 (m, 1H), 4.57 (t, J=8.4 Hz, 1H), 4.43-4.41 (m, 1H), 4.38 (t, J=4.8 Hz, 1H), 4.21 (t, J=4.8 Hz, 1H), 3.88 (d, J=10.8 Hz, 1H), 3.74 (dd, J=10.8 Hz, J=4.0 Hz, 1H), 3.64 (t, J=4.8 Hz, 1H), 3.53 (t, J=4.8 Hz, 1H), 3.38-3.34 (m, 1H), 3.31-3.17 (m, 3H), 2.49-2.42 (m, 5H), 2.30-2.19 (m, 3H), 1.99-1.95 (m, 1H), 1.70-1.56 (m, 4H), 1.50 (d, J=7.2 Hz, 3H), 1.40-1.30 (m, 9H), 1.04 (s, 9H), 0.94-0.88 (m, 3H); UPLC-MS (ESI⁺) calc. for C₅₇H₇₄N₅O₅S [M+1]⁺: 940.54, found 940.82; Purity 97.0% (HPLC).

### (2S,4R)-1-((S)-2-(8-(Ethyl(2-(4-((5-hydroxy-2-(4-hydroxyphenyl)-3-methyl-1H-indol-1-yl)methyl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (40)

This compound was prepared using a procedure similar to that used for compound 15. ¹H NMR (CD₃OD, 400 MHz) δ (ppm) 9.08 (s, 1H), 7.45-7.40 (m, 4H), 7.12 (d, J=8.4 Hz, 2H), 6.99 (d, J=8.8 Hz, 1H), 6.91 (d, J=2.4 Hz, 1H), 6.84-6.75 (m, 6H), 6.63 (dd, J=8.8 Hz, J=2.4 Hz, 1H), 5.11 (s, 2H), 5.02-4.97 (m, 1H), 4.64-4.62 (m, 1H), 4.56 (t, J=8.4 Hz, 1H), 4.43-4.41 (m, 1H), 4.24 (t, J=4.8 Hz, 1H), 3.87 (d, J=11.2 Hz, 1H), 3.73 (dd, J=11.2 Hz, J=4.0 Hz, 1H), 3.54 (t, J=4.8 Hz, 1H), 2.49 (s, 3H), 2.31-2.15 (m, 6H), 1.98-1.92 (m, 1H), 1.73-1.65 (m, 2H), 1.59-1.55 (m, 2H), 1.50 (d, J=7.2 Hz, 3H), 1.40-1.29 (m, 9H), 1.03 (s, 9H); ¹³C NMR (CD₃OD, 100 MHz) δ (ppm) 175.91, 173.21, 172.33, 158.52, 157.92, 153.66, 151.65, 146.06, 139.86, 133.87, 133.08, 132.79, 131.04, 130.50, 128.68, 127.72, 124.47, 116.23, 115.54, 112.17, 111.59, 108.66, 103.92, 70.94, 63.27, 60.60, 59.00, 57.98, 54.43, 52.72, 50.15, 47.58, 38.78, 36.47, 29.90, 29.80, 29.76, 27.04, 24.69, 22.35, 15.21, 9.62, 9.05; UPLC-MS (ESI⁺) calc. for C₅₇H₇₃N₆O₇S [M+1]⁺: 985.53, found 985.82; Purity >99.5% (HPLC).

### (2S,4R)-1-((S)-2-(8-(Ethyl(2-(4-((1R,2S)-6-hydroxy-2-phenyl-1,2,3,4-tetrahydronaphthalen-1-yl)phenoxy)ethyl)amino)octanamido)-3,3-dimethylbutanoyl)-4-hydroxy-N—((S)-1-(4-(4-methylthiazol-5-yl)phenyl)ethyl)pyrrolidine-2-carboxamide (41)

This compound was prepared using a procedure similar to that used for compound 15. ¹H NMR (CD₃OD, 400 MHz) δ (ppm) 8.94 (s, 1H), 7.46-7.35 (m, 4H), 7.13-7.09 (m, 3H), 6.83-6.80 (m, 2H), 6.68-6.61 (m, 4H), 6.52 (dd, J=8.4 Hz, J=4.0 Hz 1H), 6.38 (d, J=8.4 Hz, 2H), 5.03-4.98 (m, 1H), 4.64-4.54 (m, 2H), 4.43-4.41 (m, 1H), 4.25-4.20 (m, 3H), 3.89-3.86 (m, 1H), 3.75 (dd, J=11.2 Hz, J=4.0 Hz, 1H), 3.56-3.53 (m, 2H), 3.37-3.35 (m, 1H), 3.23-3.16 (m, 2H), 3.06-2.99 (m, 2H), 2.49 (s, 3H), 2.34-2.14 (m, 4H), 1.99-1.92 (m, 1H), 1.79-1.50 (m, 8H), 1.38-1.29 (m, 9H), 1.03 (s, 9H); ¹³C NMR (CD₃OD, 100 MHz) δ (ppm) 175.89, 173.23, 172.31, 157.05, 156.64, 153.10, 148.68, 145.79, 138.82, 137.76, 132.43, 132.06, 131.33, 130.51, 129.21, 128.72, 127.66, 127.66, 127.44, 126.97, 126.63, 115.50, 114.68, 113.98, 70.96, 63.16, 61.04, 60.61, 58.98, 58.01, 54.48, 52.81, 52.24, 51.57, 50.15, 46.73, 38.82, 36.52, 36.49, 30.97, 29.97, 28.83, 27.38, 27.05, 26.74, 25.59, 24.73, 24.44, 23.24, 22.37, 15.62, 9.06; UPLC-MS (ESI⁺) calc. for C₅₇H₇₄N₅O₆S [M+1]⁺: 956.54, found 956.48.

### (2S,4R)-1-((S)-2-(8-(Ethyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N-(4-(4-methylthiazol-5-yl)benzyl)pyrrolidine-2-carboxamide (42)

This compound was prepared using a procedure similar to that used for compound 15. ¹H NMR (CD₃OD, 400 MHz) δ (ppm) 8.86 (s, 1H), 7.74 (d, J=9.2 Hz, 2H), 7.46-7.39 (m, 5H), 7.27 (d, J=2.4 Hz, 1H), 7.18 (d, J=8.8 Hz, 2H), 6.91 (d, J=8.8 Hz, 2H), 6.87 (dd, J=8.8 Hz, J=2.0 Hz, 1H), 6.61 (d, J=8.8 Hz, 2H), 4.64 (d, J=8.8 Hz, 1H), 4.58-4.49 (m, 3H), 4.38-4.33 (m, 3H), 3.90 (d, J=11.2 Hz, 1H), 3.80 (dd, J=10.8 Hz, J=4.0 Hz, 1H), 3.59 (t, J=4.8 Hz, 2H), 3.21-3.17 (m, 2H), 2.46 (s, 3H), 2.32-2.19 (m, 3H), 2.11-2.03 (m, 1H), 1.73-1.71 (m, 2H), 1.62-1.59 (m, 2H), 1.38-1.29 (m, 9H), 1.02 (s, 9H); UPLC-MS (ESI⁺) calc. for C₅₅H₆₆N₅O₈S₂ [M+1]⁺: 988.44, found 988.98; Purity 97.8% (HPLC).

### (2S,4R)—N—((S)-1-(4-Chlorophenyl)ethyl)-1-((S)-2-(8-(ethyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxypyrrolidine-2-carboxamide (43)

This compound was prepared using a procedure similar to that used for compound 15. ¹H NMR (CD₃OD, 400 MHz) δ (ppm) 7.75 (d, J=8.8 Hz, 2H), 7.44 (d, J=8.8 Hz, 1H), 7.32-7.27 (m, 5H), 7.21-7.15 (m, 2H), 6.95-6.86 (m, 3H), 6.65-6.60 (m, 2H), 4.95-4.87 (m, 1H), 4.63-4.61 (m, 1H), 4.53 (t, J=8.4 Hz, 2H), 3.87-3.84 (m, 1H), 3.73 (dd, J=11.2 Hz, J=4.0 Hz, 1H), 3.64-3.54 (m, 3H), 3.26-3.16 (m, 3H), 3.07-2.96 (m, 1H), 2.33-2.13 (m, 3H), 1.95-1.88 (m, 1H), 1.78-1.58 (m, 5H), 1.51 (d, J=7.2 Hz, 2H), 1.41-1.29 (m, 11H), 1.02 (s, 9H); ¹³C NMR (CD₃OD, 100 MHz) δ (ppm) 195.37, 175.91, 173.18, 172.29, 163.09, 159.28, 156.88, 144.38, 144.13, 141.49, 134.22, 133.71, 133.53, 132.77, 131.65, 131.47, 131.02, 129.56, 128.70, 128.70, 128.47, 126.04, 124.71, 116.59, 124.71, 116.59, 116.47, 116.13, 115.41, 107.91, 70.94, 63.57, 60.57, 58.99, 57.98, 54.55, 52.59, 50.24, 49.82, 38.78, 36.49, 34.62, 29.95, 29.81, 27.33, 27.03, 26.73, 25.75, 24.71, 22.28, 9.06; UPLC-MS (ESI⁺) calc. for C₅₂H₆₄ClN₄O₈S [M+1]⁺: 939.41, found 939.45.

### (2S,4R)—N—((S)-1-(4-Chlorophenyl)ethyl)-1-((S)-2-(8-(ethyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxypyrrolidine-2-carboxamide (44)

This compound was prepared using a procedure similar to that used for compound 15. ¹H NMR (CD₃OD, 400 MHz) δ (ppm) 7.75 (d, J=8.8 Hz, 2H), 7.68 (d, J=8.8 Hz, 2H), 7.49-7.40 (m, 3H), 7.27 (d, J=2.4 Hz, 1H), 7.18 (d, J=8.8 Hz, 2H), 6.94-6.86 (m, 3H), 6.64-6.60 (m, 2H), 5.01-4.96 (m, 1H), 4.63-4.61 (m, 1H), 4.47-4.38 (m, 1H), 4.35 (t, J=4.8 Hz, 2H), 3.88-3.85 (m, 1H), 3.72 (dd, J=11.2 Hz, J=4.0 Hz, 1H), 3.60-3.58 (m, 2H), 3.23-3.17 (m, 2H), 2.32-2.16 (m, 3H), 1.94-1.87 (m, 1H), 1.73-1.60 (m, 5H), 1.54-1.46 (m, 3H), 1.38-1.29 (m, 10H), 1.02 (s, 9H); ¹³C NMR (CD₃OD, 100 MHz) δ (ppm) 195.36, 175.91, 173.38, 172.30, 163.09, 159.28, 156.88, 151.22, 144.39, 141.49, 134.22, 131.47, 131.02, 128.06, 127.85, 126.04, 124.71, 119.67, 116.48, 116.13, 115.40, 111.78, 107.91, 70.95, 63.56, 60.51, 58.99, 57.99, 54.54, 52.58, 50.33, 50.22, 38.81, 36.49, 29.96, 29.82, 27.34, 27.02, 26.73, 24.72, 22.09, 9.06; UPLC-MS (ESI⁺) calc. for C₅₃H₆₄N₅O₈S [M+1]⁺: 930.45, found 930.48.

### (2S,4R)-1-((S)-2-(8-(Ethyl(2-(4-(6-hydroxy-2-(4-hydroxyphenyl)benzo[b]thiophene-3-carbonyl)phenoxy)ethyl)amino)octanamido)-3,3-dimethylbutanoyl)-N—((S)-1-(4-ethynylphenyl)ethyl)-4-hydroxypyrrolidine-2-carboxamide (45)

This compound was prepared using a procedure similar to that used for compound 15. ¹H NMR (CD₃OD, 400 MHz) δ (ppm) 7.75 (d, J=8.8 Hz, 2H), 7.46-7.38 (m, 3H), 7.29-7.22 (m, 3H), 7.19-7.15 (m, 2H), 6.93-6.86 (m, 3H), 6.61 (d, J=8.8 Hz, 2H), 4.97-4.92 (m, 1H), 4.63-4.61 (m, 1H), 4.54 (t, J=8.4 Hz, 2H), 4.42-4.39 (m, 1H), 4.36 (t, J=4.8 Hz, 2H), 3.88-3.85 (m, 1H), 3.75-3.71 (m, 2H), 3.63-3.58 (m, 3H), 3.44 (s, 1H), 3.26-3.17 (m, 3H), 2.33-2.14 (m, 4H), 1.95-1.89 (m, 1H), 1.80-1.65 (m, 3H), 1.67-1.54 (m, 3H), 1.46-1.30 (m, 10H), 1.02 (s, 9H); ¹³C NMR (CD₃OD, 100 MHz) δ (ppm) 195.38, 175.91, 173.20, 172.29, 163.10, 161.00, 159.28, 156.88, 155.70, 146.12, 144.39, 141.49, 134.22, 133.53, 133.18, 132.77, 131.47, 131.02, 130.05, 129.93, 127.14, 126.04, 124.71, 122.43, 116.47, 116.12, 115.41, 107.90, 84.19, 78.50, 70.95, 64.27, 60.58, 59.62, 58.99, 57.98, 55.93, 55.83, 55.65, 54.56, 53.65, 52.59, 50.17, 36.49, 29.94, 29.81, 27.03, 26.73, 24.71, 22.23, 18.70, 17.26, 13.17, 9.07; UPLC-MS (ESP) calc. for C₅₃H₆₄N₅O₈S [M+1]⁺: 929.45, found 929.49.

### (2S,4R)—N—((S)-1-(4-Cyclopropylphenyl)ethyl)-1-((S)-2-(8-(ethyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxypyrrolidine-2-carboxamide (46)

This compound was prepared using a procedure similar to that used for compound 15. ¹H NMR (CD₃OD, 400 MHz) δ (ppm) 7.76 (d, J=8.8 Hz, 2H), 7.45 (d, J=8.8 Hz, 1H), 7.27 (d, J=2.0 Hz, 2H), 7.20-7.10 (m, 4H), 7.02-6.98 (m, 2H), 6.94-6.86 (m, 3H), 6.63-6.59 (m, 2H), 4.95-4.91 (m, 1H), 4.63-4.61 (m, 1H), 4.54 (t, J=8.4 Hz, 1H), 4.43-4.39 (m, 1H), 4.36-4.34 (m, 2H), 3.87-3.85 (m, 1H), 3.75-3.71 (m, 1H), 3.63-3.58 (m, 2H), 3.23-3.17 (m, 2H), 2.31-2.23 (m, 2H), 2.18-2.11 (m, 1H), 1.96-1.83 (m, 2H), 1.72-1.59 (m, 4H), 1.51-1.28 (m, 12H), 1.02 (s, 9H), 1.00-0.90 (m, 2H), 0.64-0.60 (m, 2H); ¹³C NMR (CD₃OD, 100 MHz) δ (ppm) 195.36, 175.88, 172.98, 172.30, 163.07, 159.29, 156.90, 144.39, 144.17, 142.11, 141.50, 134.22, 133.54, 131.48, 131.02, 126.95, 126.72, 126.06, 124.70, 116.47, 116.13, 115.40, 107.90, 70.94, 63.55, 60.64, 58.98, 57.98, 55.90, 54.57, 53.58, 52.62, 51.85, 51.29, 38.76, 36.51, 29.93, 29.83, 27.36, 27.04, 26.72, 24.72, 15.77, 9.51, 9.06; UPLC-MS (ESP) calc. for C₅₅H₆₉N₄O₈S [M+1]⁺: 945.48, found 945.51.

### (2S,4R)-1-((S)-2-(8-(Ethyl(2-(4-(6-hydroxy-2-(4-hydroxyphenyl)benzo[b]thiophene-3-carbonyl)phenoxy)ethyl)amino)octanamido)-3,3-dimethylbutanoyl)-4-hydroxy-N—((S)-1-(4-isopropylphenyl)ethyl)pyrrolidine-2-carboxamide (47)

This compound was prepared using a procedure similar to that used for compound 15. ¹H NMR (CD₃OD, 400 MHz) δ (ppm) 7.75 (d, J=8.8 Hz, 2H), 7.44 (d, J=8.8 Hz, 2H), 7.27 (d, J=2.0 Hz, 1H), 7.22-7.15 (m, 6H), 6.93-6.86 (m, 3H), 6.63-6.59 (m, 2H), 4.96-4.91 (m, 1H), 4.62 (d, J=8.8 Hz, 1H), 4.53 (d, J=8.4 Hz, 1H), 4.43-4.36 (m, 1H), 4.35-4.29 (m, 2H), 3.87-3.84 (m, 1H), 3.75-3.71 (m, 1H), 3.60-3.53 (m, 2H), 3.24-3.15 (m, 2H), 2.89-2.81 (m, 1H), 2.34-2.21 (m, 2H), 2.18-2.12 (m, 1H), 1.98-1.91 (m, 1H), 1.73-1.70 (m, 2H), 1.63-1.58 (m, 2H), 1.52-1.29 (m, 12H), 1.22 (d, J=7.2 Hz, 6H), 1.02 (s, 9H); ¹³C NMR (CD₃OD, 100 MHz) δ (ppm) 195.35, 175.89, 173.00, 172.30, 163.08, 159.29, 156.88, 148.92, 144.37, 142.57, 141.49, 134.22, 133.53, 132.79, 131.47, 131.01, 127.50, 126.76, 126.03, 124.71, 116.13, 115.40, 107.91, 70.94, 63.55, 60.63, 58.98, 57.98, 54.53, 52.57, 50.20, 50.10, 38.76, 36.51, 35.06, 29.94, 29.81, 27.34, 27.04, 26.72, 24.70, 24.45, 22.49, 9.06; UPLC-MS (ESI⁺) calc. for C₅₅H₇₁N₄O₈S [M+1]⁺: 947.50, found 947.53.

### (2S,4R)—N—((S)-1-(4-(tert-Butyl)phenyl)ethyl)-1-((S)-2-(8-(ethyl(2-(4-(6-hydroxy-2-(4-hydroxyphenyl)benzo[b]thiophene-3-carbonyl)phenoxy)ethyl)amino) octanamido)-3,3-dimethylbutanoyl)-4-hydroxypyrrolidine-2-carboxamide (48)

This compound was prepared using a procedure similar to that used for compound 15. ¹H NMR (CD₃OD, 400 MHz) δ (ppm) 7.75 (d, J=8.8 Hz, 2H), 7.45 (d, J=8.8 Hz, 1H), 7.34 (d, J=8.4 Hz, 2H), 7.27 (d, J=2.0 Hz, 1H), 7.22-7.16 (m, 4H), 6.93-6.86 (m, 3H), 6.63-6.59 (m, 2H), 4.95-4.91 (m, 1H), 4.65-4.60 (m, 1H), 4.53 (t, J=8.4 Hz, 1H), 4.42-4.40 (m, 1H), 4.35 (t, J=4.4 Hz, 2H), 3.87-3.84 (m, 1H), 3.75-3.71 (m, 1H), 3.60-3.56 (m, 2H), 3.25-3.16 (m, 2H), 2.34-2.14 (m, 3H), 1.98-1.91 (m, 1H), 1.74-1.71 (m, 2H), 1.64-1.55 (m, 2H), 1.45-1.32 (m, 1H), 1.29 (s, 9H), 1.02 (s, 9H); ¹³C NMR (CD₃OD, 100 MHz) δ (ppm) 193.36, 173.88, 171.01, 170.31, 161.08, 157.29, 154.88, 149.06, 142.37, 140.13, 139.49, 132.22, 131.53, 130.80, 129.47, 129.02, 124.71, 124.48, 124.39, 124.04, 122.70, 114.47, 114.13, 113.40, 105.91, 68.95, 61.56, 58.64, 56.98, 55.98, 52.54, 50.58, 48.21, 48.02, 36.77, 34.51, 33.26, 29.78, 27.94, 27.81, 25.34, 25.04, 24.72, 22.70, 20.45, 7.06; UPLC-MS (ESI⁺) calc. for C₅₆H₇₃N₄O₈S [M+1]⁺: 961.51, found 961.55.

### Example 2

### Biological Assays

Cell Culture. Human breast cancer cell lines MCF-7 (ATCC® HTB-22™) and T47D (ATCC® HTB-133™) were purchased from the American Type Culture Collection (ATCC), Manassas, Va., and maintained and cultured in Dulbecco's Modified Eagle's medium (DMEM) containing 10% fetal bovine serum, 1 unit/ml of penicillin and 1 μg/ml of streptomycin. Cells with 3-8 passages after purchase were used in experiments as indicated.

Western Blot Analysis. Western blot analysis was performed essentially as described previously (Hu et al, 2015, PMID: 26358219). Cells treated with indicated compounds were lysed in Radioimmunoprecipitation Assay Protein Lysis and Extraction Buffer (25 mmol/L Tris.HCl, pH 7.6, 150 mmol/L NaCl, 1% Nonidet P-40, 1% sodium deoxycholate, and 0.1% sodium dodecyl sulfate) containing proteinase inhibitor cocktail (Roche Diagnostics, Mannheim, Germany). After determination of protein concentration by BCA assay (Fisher Scientific, Pittsburgh, Pa.), equal amounts of total protein were electrophoresed through 10% SDS-polyacrylamide gels. The separated protein bands were transferred onto PVDF membranes (GE Healthcare Life Sciences, Marlborough, Mass.) and blotted against different antibodies, as indicated. The human estrogen receptor a antibodies (AB16460) were purchased from Abeam, Inc., Cambridge, Mass. The membranes were reblotted with horseradish peroxidase-conjugated anti-glyceraldehyde-3-phosphate dehydrogenase antibody (G9295) from Sigma-Aldrich Corporation, St. Louis, Mo. The blots were scanned and the band intensities were quantified using GelQuant.NET software as described in biochemlabsolutions.com. The relative mean intensity of target proteins was expressed after normalization to the intensity of glyceraldehyde-3-phosphate dehydrogenase bands from individual repeats.

Cell Growth Assay. Cells were seeded at 1500/well in 96 well plates overnight. One day after seeding, they were treated with indicated doses of compounds. The growth of the cells was evaluated by colorimetric WST-8 assay 4 days after the compound treatment following the instructions of the manufacturer, Cayman Chemical, Ann Arbor, Mich.

Molecular Modeling. The binding pose of the N,N-diethylamino analogue of raloxifene in a complex with ER was modelled with the structure (PDB:1ERR)⁴⁹ co-crystallized with raloxifene using the MOE program. If atoms were missing, residues were rebuilt based on the amber 10 library in MOE and protons were added using the “protonate 3D” module considering by setting pH at 7, the temperature at 300 K and the salt concentration at 0.1 mol/L. Docking simulations were then performed using raloxifene to define the binding site with crystallized H₂O molecules preserved. The ligand was placed by “Triangle matcher” and evaluated by London dG scoring. DGVI/WSA dG scoring was then applied to rank the poses, and the top ranked pose was selected. Figures appeared in this paper were prepared using the PyMOL program available on the world wide web at pymol.org.

Cloning and Purification of VHL-ElonginBC complex. The DNA sequence of VHL (coding for residues 54-213) was constructed by PCR and inserted into a His-TEV expression vector⁵⁸ using ligation-independent cloning. The DNA sequences of Elongin B (encoding residues 1-118) and Elongin C (encoding residues 1-96) were constmcted by PCR and inserted into pCDFDuet 1 using Gibson assembly.⁵⁹ BL21(DE3) cells were transformed simultaneously with both plasmids and grown in Terrific Broth at 37° C. until an OD600 of 1.2. The cells were induced overnight with 0.4 mM IPTG at 24° C. Pelleted cells were freeze-thawed then resuspended in 20 mM Tris HCl pH7.0, 200 mM NaCl and 0.1% β-mercaptoethanol (bME) containing protease inhibitors. The cell suspension was lysed by sonication and debris removed via centrifugation. The supernatant was incubated at 4° C. for 1 hr with Ni-NTA (Qiagen) pre-washed in 20 mM Tris-HCl pH 7.0, 200 mM NaCl and 10 mM Imidazole. The protein complex was eluted in 20 mM Tris-HCl pH 7.0, 200 mM NaCl and 300 mM Imidazole, dialyzed into 20 mM Tris-HCl pH 7.0, 150 mM NaCl, and 0.01% bME and incubated with TEV protease overnight at 4° C. The protein sample was reapplied to the Ni-NTA column to remove the His-tag. The flow through containing the VHL complex was diluted to 75 mM NaCl and applied to a HiTrap Q column (GE Healthcare). The sample was eluted with a salt gradient (0.075-1 M NaCl), concentrated and further purified on a Superdex S75 column (GE Healthcare) pre-equilibrated with 20 mM Bis-Tris 7.0, 150 mM NaCl and 1 mM DTT. Samples were aliquoted and stored at −80° C.

Binding Affinities of VHL ligands to VHL. A fluorescence-polarization (FP) competitive assay was established using VHL-ElonginBC complex and a fluorescently tagged probe (SI). The IC₅₀ and Kᵢ values of VHL ligands were determined in competitive binding experiments. Mixtures of 5 μL of compounds in DMSO and 95 μL of preincubated protein/tracer complex solution were added into assay plates which were incubated at rt for 60 min with gentle shaking. Final concentrations of VHL-ElonginBC complex and fluorescent probe were both 5 nM. Negative controls containing protein/probe complex only (equivalent to 0% inhibition) and positive controls containing only free probes (equivalent to 100% inhibition) were included in each assay plate. FP values in millipolarization units (mP) were measured using the Infinite M-1000 plate reader (Tecan U.S., Research Triangle Park, N.C.) in Microfluor 1 96-well, black, round-bottom plates (Thermo Scientific, Waltham, Mass.) at an excitation wavelength of 485 nm and an emission wavelength of 530 nm. IC₅₀ values were determined by nonlinear regression fitting of the competition curves. Kᵢ values of competitive inhibitors were obtained directly by nonlinear regression fitting, based upon the K_{D} values of the probe and concentrations of the protein and probe in the competitive assays. All the FP competitive experiments were performed in duplicate in three independent experiments.

### Example 3

### Biological Assay Results

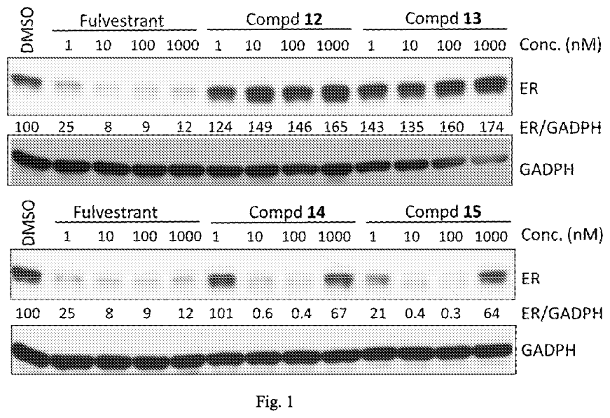

Representative Compounds of the Disclosure were evaluated for their ability to induce ER degradation in the MCF-7 ER+ breast cancer cell line, with fulvestrant used as the control. Western blotting data for compounds 12-15 are shown in FIG. 1.

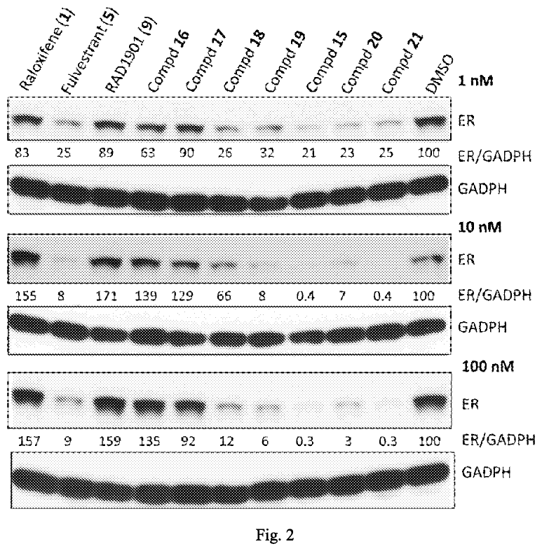

Representative Compounds of the Disclosure with the linker length varying from 3 to 9 atoms were evaluated for their ability to induce ER degradation in MCF-7 cells at concentrations of 1 nM, 10 nM and 100 nM, with compound 15, fulvestrant (5), RAD1901 (9), and raloxifene (1) included as controls. Western blotting data is shown in FIG. 2. Compounds 15, 18, 19, 20, and 21, with linkers containing 6-9 carbon atoms, were surprisingly effective in inducing ER degradation at concentrations as low as 1 nM.

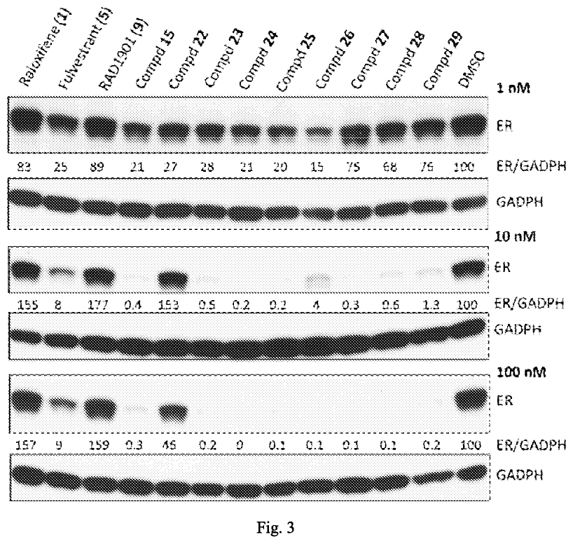

Representative Compounds of the Disclosure with various R³ groups were evaluated for their ability to induce ER degradation in MCF-7 cells at concentrations of 1 nM, 10 nM and 100 nM, with compound 15, fulvestrant (5), RAD1901 (9), and raloxifene (1) included as controls. Western blotting data is shown in FIG. 3.

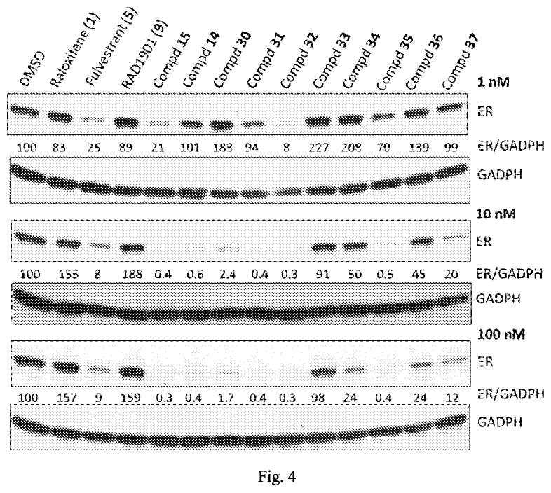

Representative Compounds of the Disclosure with various linkers were evaluated for their ability to induce ER degradation in MCF-7 cells at concentrations of 1 nM, 10 nM and 100 nM, with compound 15, fulvestrant (5), RAD1901 (9), and raloxifene (1) included as controls. Western blotting data is shown in FIG. 4.

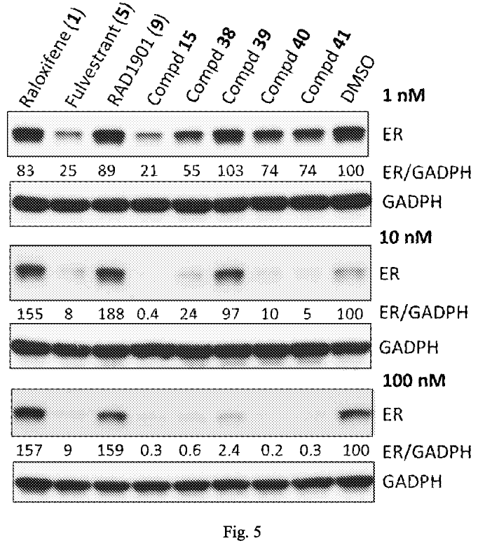

Representative Compounds of the Disclosure with various estrogen receptor modulators were evaluated for their ability to induce ER degradation in MCF-7 cells at concentrations of 1 nM, 10 nM and 100 nM, with compound 15, fulvestrant (5), RAD1901 (9), and raloxifene (1) included as controls. Western blotting data is shown in FIG. 5.

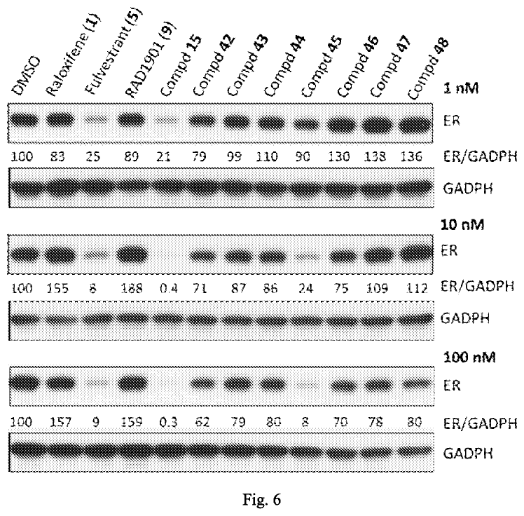

Representative Compounds of the Disclosure, see Table 1, with various E3 ligase ligands were evaluated for their ability to induce ER degradation in MCF-7 cells at concentrations of 1 nM, 10 nM and 100 nM, with compound 15, fulvestrant (5), RAD1901 (9), and raloxifene (1) included as controls. Western blotting data is shown in FIG. 6.

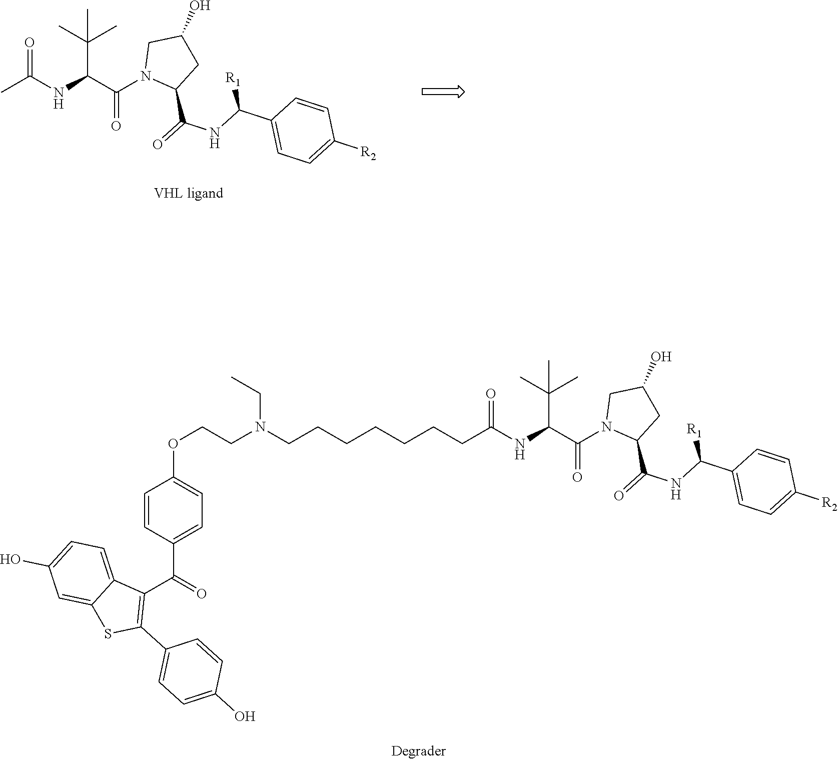

A fluorescence polarization (FP) assay for VHL was used to determine the binding affinities of VHL ligands 11 and 43a-48a, with a previously reported VHL ligand (VH032)⁵⁴ included as a control. These results are presented in Table 1.

TABLE 1

| VHL | FP Binding affinity | | | |
|---|---|---|---|---|
| ligand | IC₅₀ (nM) | Degrader | R₁ | R₂ |
| 11 VH032 | 196 ± 6 454 ± 24 | 15 (ERD-148) 42 | Me H | 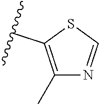 [SMILES: *c1scnc1C] |
| 43a | 7867 ± 642 | 43 | Me | Cl |
| 44a | 4827 ± 418 | 44 | Me | CN |
| 45a | 879 ± 97 | 45 | Me | ethynyl |
| 46a | 6112 ± 820 | 46 | Me | c-Pr |
| 47a | 41001 ± 6665 | 47 | Me | i-pr |
| 48a | 58897 ± 5264 | 48 | Me | t-Bu |

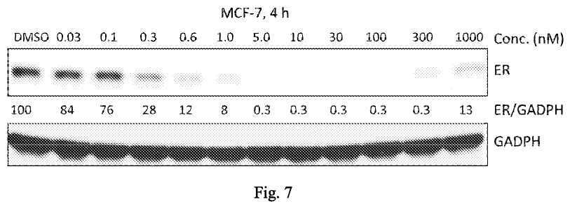

The ER degradation by compound 32 in a wide range of concentrations to determine its DC₅₀ (concentration to achieve 50% of protein degradation) in MCF-7 cells was tested. See FIG. 7 Quantification of the Western blotting data showed that compound 32 achieves a DC₅₀ value of 0.17 nM in the MCF-7 cells with a 4 h treatment time. Compound 32 achieves a maximum ER degradation of >95% based upon quantification at concentrations as low as 5 nM.

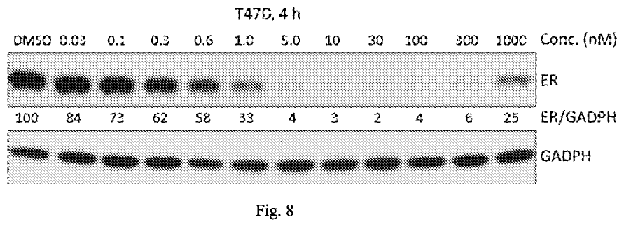

Compound 32 was also evaluated for its ability to induce ER degradation in the T47D ER+ breast cancer cell line. As shown in FIG. 8, compound 32 achieves a DC₅₀ value of 0.43 nM and a maximum degradation of >95% at 5 nM. Compound 32 at 1 μM also demonstrates a hook effect in the T47D cells.

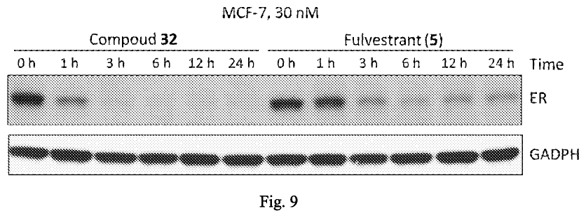

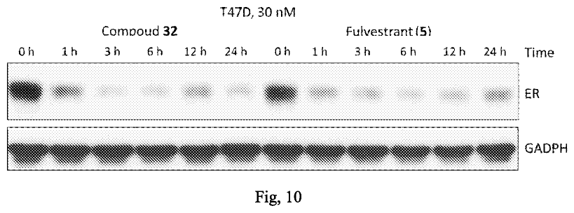

The kinetics of ER degradation induced by compound 32 in MCF-7 cells was examined. As shown in FIG. 9, at a concentration of 30 nM, compound 32 reduces >80% of the ER protein level with a 1 h treatment and achieves essentially complete ER degradation at the 3 h time-point, indicating fast kinetics. In comparison, fulvestrant, has only a modest effect on reduction of the level of ER at 1 h and achieves a maximum of approximately 90% of ER degradation after a 24 h treatment. The kinetic data obtained for 32 and fulvestrant in the T47D cells were similar to those observed in MCF-7 cells See FIG. 10.

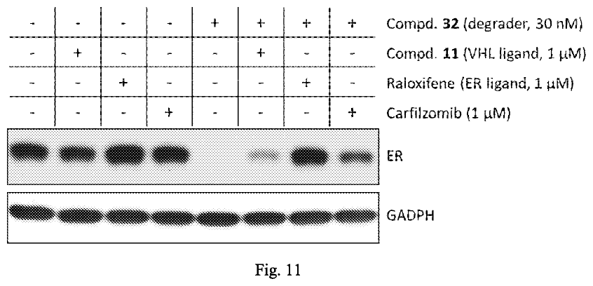

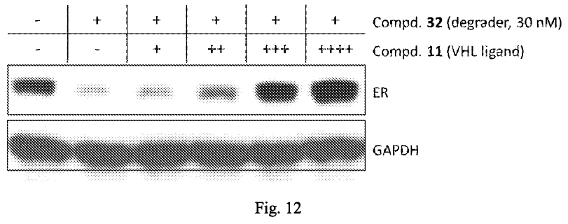

The mechanism of action of ER degradation induced by 32 was investigated. ER degradation induced by compound 32 at a 30 nM concentration is significantly reduced by addition of 1 μM of raloxifene or 1 μM of the proteasome inhibitor carfilzomib, but raloxifene or carfilzomib alone have no effect on the ER protein levels. See FIG. 11. Interestingly, 1 μM of the VHL ligand (11) blocks the degradation by 30 nM of compound 32 only slightly (FIG. 11). To further confirm that the degradation is VHL-dependent, a dose-response experiment with VHL ligand 11 was performed. As shown in FIG. 12, the degradation by compound 32 was completely blocked with 5 μM or 10 μM of 11.

A WST-8 cell proliferation assay was used to evaluate the ability of compound 32 to inhibit cell proliferation in MCF-7 cells, with raloxifene and fulvestrant included as controls (data not shown). Compound 32 is achieves an IC₅₀ value of 0.77 nM and a maximum inhibition (Imax) of 57.5% in MCF-7 cells. Fulvestrant achieves an Imax value of 43.8%. Raloxifene achieves an Imax value of 34.0%. RAD1901, a previously reported SERD molecule¹⁸, achieves an Imax value of 25.7%. Compound 32 does not exhibit the cell proliferation inhibition effects in triple-negative breast cancer cell MDA-MB-231 and primary human mammary epithelial cells.

To visually evaluate the cellular effect, a crystal violet staining experiment was used to test compound 32 at 10 nM, 100 nM and 300 nM with raloxifene and fulvestrant as controls (data not shown). Consistent with the WST-8 cell proliferation assay, treatment of MCF-7 cells with compound 32 reduced cell proliferation more significantly than raloxifene or fulvestrant at all three of the concentrations tested

A quantitative reverse transcription-polymerase chain reaction (qRT-PCR) analysis was used to evaluate the ability of compound 32 to suppress the mRNA levels of pGR and GREB1, two ER-regulated genes in MCF-7 cells (data not shown). The expression of both genes is strongly suppressed by compound 32. Compound 32 is slightly more effective than fulvestrant in suppressing the expression of pGR and GREB1 at both 10 nM and 100 nM. Compound 32 is significantly more effective than raloxifene in suppressing the expression of pGR and GREB1 at both 10 nM and 100 nM.

### Example 4

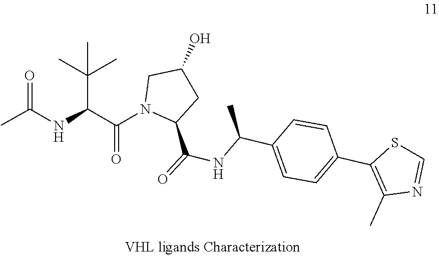 [SMILES: CC(=O)N[C@H](C(=O)N1C[C@H](O)C[C@H]1C(=O)N[C@@H](C)c1ccc(-c2scnc2C)cc1)C(C)(C)C]

(2S,4R)-1-((S)-2-acetamido-3,3-dimethylbutanoyl)-4-hydroxy-N—((S)-1-(4-(4-methylthiazol-5-yl)phenyl)ethyl)pyrrolidine-2-carboxamide (11): ¹H NMR (CD₃OD, 400 MHz) δ (ppm) 9.02 (s, 1H), 7.47-7.42 (m, 4H), 5.04-4.98 (m, 1H), 4.62-4.55 (m, 2H), 4.43-4.41 (m, 1H), 3.88 (d, J=10.8 Hz, 1H), 3.74 (dd, J=10.8 Hz, J=4.0 Hz, 1H), 2.50 (s, 3H), 2.22-2.16 (m, 1H), 2.00 (s, 3H), 1.98-1.91 (m, 1H), 1.51 (d, J=6.8 Hz, 3H), 1.05 (s, 9H); ¹³C NMR (CD₃OD, 100 MHz) δ (ppm) 173.26, 173.11, 172.28, 153.34, 148.20, 146.01, 133.91, 131.04, 130.51, 127.69, 127.52, 70.97, 60.55, 59.22, 57.97, 50.14, 38.77, 36.41, 26.99, 22.38, 22.29, 15.41; UPLC-MS (ESI⁺) calculated for C₂₅H₃₅N₄O₄S [M+1]⁺: 487.24, found 487.43.

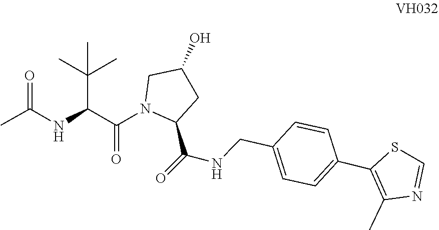 [SMILES: CC(=O)N[C@H](C(=O)N1C[C@H](O)C[C@H]1C(=O)NCc1ccc(-c2scnc2C)cc1)C(C)(C)C]

(2S,4R)-1-((S)-2-acetamido-3,3-dimethylbutanoyl)-4-hydroxy-N-(4-(4-methylthiazol-5-yl)benzyl)pyrrolidine-2-carboxamide (VH032): ¹H NMR (CD₃OD, 400 MHz) δ (ppm) 9.59 (s, 1H), 7.53-7.45 (m, 4H), 4.61-4.51 (m, 4H), 4.38 (d, J=15.6 Hz, 1H), 3.92 (d, J=10.8 Hz, 1H), 3.80 (dd, J=10.8 Hz, J=4.0 Hz, 1H), 2.54 (s, 3H), 2.26-2.21 (m, 1H), 2.12-2.05 (m, 1H), 2.00 (s, 3H), 1.03 (s, 9H); ¹³C NMR (CD₃OD, 100 MHz) δ (ppm) 174.50, 173.12, 172.31, 155.25, 152.15, 141.76, 130.38, 129.89, 129.24, 122.18, 71.06, 60.80, 59.21, 57.97, 43.61, 38.89, 36.42, 26.95, 22.31, 13.84; UPLC-MS (ESI⁺) calculated for C₂₄H₃₃N₄O₄S [M+1]⁺: 473.22, found 473.07.

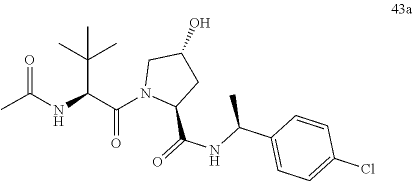 [SMILES: CC(=O)N[C@H](C(=O)N1C[C@H](O)C[C@H]1C(=O)N[C@@H](C)c1ccc(Cl)cc1)C(C)(C)C]

(2S,4R)-1-((S)-2-acetamido-3,3-dimethylbutanoyl)-N—((S)-1-(4-chlorophenyl)ethyl)-4-hydroxypyrrolidine-2-carboxamide (43a): ¹H NMR (CD₃OD, 400 MHz) δ (ppm) 7.30-7.28 (m, 4H), 4.94 (q, J=6.8 Hz, 1H), 4.61 (s, 1H), 4.56-4.51 (m, 1H), 4.43-4.41 (m, 1H), 3.86 (d, J=11.2 Hz, 1H), 3.73 (dd, J=11.2 Hz, 0.7=4.0 Hz, 1H), 2.19-2.13 (m, 1H), 2.00 (s, 3H), 1.95-1.88 (m, 1H), 1.45 (d, J=6.8 Hz, 3H), 1.04 (s, 9H); ¹³C NMR (CD₃OD, 100 MHz) δ (ppm) 173.18, 172.19, 144.17, 133.69, 129.54, 128.68, 70.94, 60.52, 59.26, 57.94, 38.73, 36.41, 26.98, 22.28, 22.23; UPLC-MS (ESI⁺) calculated for C₂₁H₃₁ClN₃O₄ [M+1]⁺: 424.20, found 424.30.

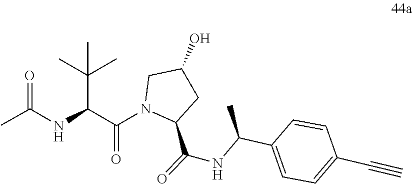 [SMILES: C#Cc1ccc([C@H](C)NC(=O)[C@@H]2C[C@@H](O)CN2C(=O)[C@@H](NC(C)=O)C(C)(C)C)cc1]

(2S,4R)-1-((S)-2-acetamido-3,3-dimethylbutanoyl)-N—((S)-1-(4-ethynylphenyl)ethyl)-4-hydroxypyrrolidine-2-carboxamide (44a): ¹H NMR (CD₃OD, 400 MHz) δ (ppm) 7.41 (d, J=8.4 Hz, 2H), 7.29 (d, J=8.4 Hz, 2H), 4.96 (q, J=6.8 Hz, 1H), 4.61 (s, 1H), 4.56-4.52 (m, 1H), 4.44-4.41 (m, 1H), 3.87 (d, J=10.8 Hz, 1H), 3.73 (dd, J=10.8 Hz, J=4.0 Hz, 1H), 3.43 (s, 1H), 2.20-2.13 (m, 1H), 2.00 (s, 3H), 1.98-1.88 (m, 1H), 1.46 (d, J=6.8 Hz, 3H), 1.04 (s, 9H); ¹³C NMR (CD₃OD, 100 MHz) δ (ppm) 173.20, 173.18, 172.20, 146.14, 133.17, 127.13, 122.41, 84.20, 78.45, 70.95, 60.52, 59.25, 57.94, 38.74, 36.41, 26.98, 22.23; UPLC-MS (ESP) calculated for C₂₃H₃₂N₃O₄ [M+1]⁺: 414.24, found 414.30.

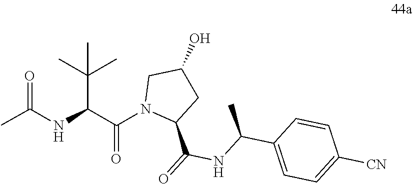 [SMILES: CC(=O)N[C@H](C(=O)N1C[C@H](O)C[C@H]1C(=O)N[C@@H](C)c1ccc(C#N)cc1)C(C)(C)C]

(2S,4R)-1-((S)-2-acetamido-3,3-dimethylbutanoyl)-N—((S)-1-(4-cyanophenyl)ethyl)-4-hydroxypyrrolidine-2-carboxamide (45a): ¹H NMR (CD₃OD, 400 MHz) δ (ppm) 7.68 (d, J=8.4 Hz, 2H), 7.48 (d, J=8.4 Hz, 2H), 4.99 (q, J=7.2 Hz, 1H), 4.60 (s, 1H), 4.55 (t, J=8.4 Hz, 1H), 4.44-4.41 (m, 1H), 3.87 (d, J=10.8 Hz, 1H), 3.73 (dd, J=10.8 Hz, J=4.0 Hz, 1H), 2.21-2.15 (m, 1H), 2.00 (s, 3H), 1.94-1.87 (m, 1H), 1.48 (d, J=7.2 Hz, 3H), 1.03 (s, 9H); ¹³C NMR (CD₃OD, 100 MHz) δ (ppm) 173.40, 173.12, 172.26, 151.27, 135.50, 128.06, 119.68, 111.76, 70.95, 60.46, 59.23, 57.96, 38.76, 36.39, 26.97, 26.94, 22.27, 22.11; UPLC-MS (ESI⁺) calculated for C₂₂H₃₁N₄O₄ [M+1]⁺: 415.23, found 415.40.

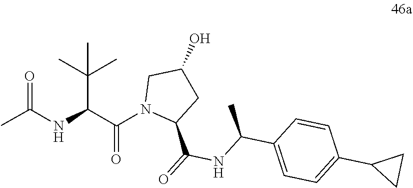 [SMILES: CC(=O)N[C@H](C(=O)N1C[C@H](O)C[C@H]1C(=O)N[C@@H](C)c1ccc(C2CC2)cc1)C(C)(C)C]

(2S,4R)-1-((S)-2-acetamido-3,3-dimethylbutanoyl)-N—((S)-1-(4-cyclopropylphenyl)ethyl)-4-hydroxypyrrolidine-2-carboxamide (46a): ¹H NMR (CD₃OD, 400 MHz) δ (ppm) 7.17 (d, J=8.0 Hz, 2H), 7.01 (d, J=8.0 Hz, 2H), 4.92-4.89 (m, 1H), 4.61 (s, 1H), 4.53 (t, J=8.4 Hz, 1H), 4.42-4.41 (m, 1H), 3.86 (d, J=10.8 Hz, 1H), 3.73 (dd, J=10.8 Hz, J=4.0 Hz, 1H), 2.17-2.11 (m, 1H), 2.00 (s, 3H), 1.97-1.83 (m, 2H), 1.44 (d, J=7.2 Hz, 3H), 1.04 (s, 9H), 0.94-0.90 (m, 2H), 0.64-0.61 (m, 2H); ¹³C NMR (CD₃OD, 100 MHz) δ (ppm) 173.15, 172.96, 172.19, 144.10, 142.13, 126.93, 126.70, 70.93, 60.54, 59.22, 57.92, 50.00, 38.68, 36.41, 26.98, 22.40, 22.25, 15.77, 9.46; UPLC-MS (ESI⁺) calculated for C₂₄H₃₆N₃O₄ [M+1]⁺: 430.27, found 430.49.

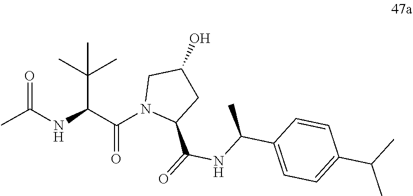 [SMILES: CC(=O)N[C@H](C(=O)N1C[C@H](O)C[C@H]1C(=O)N[C@@H](C)c1ccc(C(C)C)cc1)C(C)(C)C]

(2S,4R)-1-((S)-2-acetamido-3,3-dimethylbutanoyl)-4-hydroxy-N—((S)-1-(4-isopropylphenyl)ethyl)pyrrolidine-2-carboxamide (47a): ¹H NMR (CD₃OD, 400 MHz) δ (ppm) 7.23-7.16 (m, 4H), 4.95-4.91 (m, 1H), 4.61 (s, 1H), 4.54 (t, J=8.4 Hz, 1H), 4.43-4.41 (m, 1H), 3.86 (d, J=11.2 Hz, 1H), 3.74 (dd, J=11.2 Hz, 0.7=4.0 Hz, 1H), 2.88-2.85 (m, 1H), 2.18-2.12 (m, 1H), 2.01-1.91 (m, 4H), 1.45 (d, J=6.8 Hz, 3H), 1.22 (d, J=6.8 Hz, 6H), 1.04 (s, 9H); ¹³C NMR (CD₃OD, 100 MHz) δ (ppm) 173.09, 173.03, 172.27, 148.90, 142.63, 127.49, 127.00, 70.96, 60.57, 59.19, 57.93, 50.00, 38.71, 36.42, 35.06, 26.99, 24.44, 22.48, 22.29; UPLC-MS (ESI⁺) calculated for C₂₄H₃₈N₃O₄ [M+1]⁺: 432.29, found 432.44.

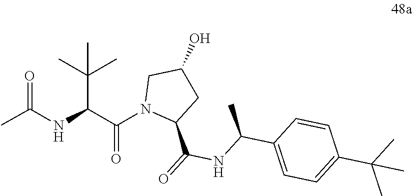 [SMILES: CC(=O)N[C@H](C(=O)N1C[C@H](O)C[C@H]1C(=O)N[C@@H](C)c1ccc(C(C)(C)C)cc1)C(C)(C)C]

(2S,4R)-1-((S)-2-acetamido-3,3-dimethylbutanoyl)-N—((S)-1-(4-(tert-butyl)phenyl)ethyl)-4-hydroxypyrrolidine-2-carboxamide (48a): ¹H NMR (CD₃OD, 400 MHz) δ (ppm) 7.35 (d, J=8.0 Hz, 2H), 7.22 (d, J=8.0 Hz, 2H), 4.93 (q, J=7.2 Hz, 1H), 4.61 (s, 1H), 4.55 (t, J=8.4 Hz, 1H), 4.43-4.41 (m, 1H), 3.87 (d, J=11.2 Hz, 1H), 3.74 (dd, J=11.2 Hz, J=4.0 Hz, 1H), 2.19-2.13 (m, 1H), 2.01-1.92 (m, 4H), 1.45 (d, J=7.2 Hz, 3H), 1.29 (s, 9H), 1.04 (s, 9H); ¹³C NMR (CD₃OD, 100 MHz) δ (ppm) 173.09, 173.02, 172.25, 151.02, 142.17, 126.71, 126.37, 70.95, 60.56, 59.19, 57.93, 50.00, 38.71, 36.42, 35.24, 31.78, 26.99, 22.46, 22.29; UPLC-MS (ESI⁺) calculated for C₂₅H₄₀N₃₀₄ [M+1]⁺: 446.30, found 446.40.

### REFERENCES

(1) Tong, C. W. S.; Wu, M.; Cho, W. C. S.; To, K. K. W. Recent advances in the treatment of breast cancer. Front. Oncol. 2018, 8.
(2) Anderson, W. F.; Katki, H. A.; Rosenberg, P. S. Incidence of breast cancer in the United States: current and future trends. J. Natl. Cancer Inst. 2011, 103, 1397-1402.
(3) Nilsson, S.; Koehler, K. F.; Gustafsson, J. A. Development of subtype-selective oestrogen receptor-based therapeutics. Nat. Rev. Drug Discovery 2011, 10, 778-792.
(4) Jordan, V. C. Tamoxifen: a most unlikely pioneering medicine. Nat. Rev. Drug Discovery 2003, 2, 205-213.
(5) Das, S.; Crockett, J. C. Osteoporosis—a current view of pharmacological prevention and treatment. Drug Des. Devel. Ther. 2013, 7, 435-448.
(6) De Marchi, T.; Foekens, J. A.; Umar, A.; Martens, J. W. Endocrine therapy resistance in estrogen receptor (ER)-positive breast cancer. Drug Discovery Today 2016, 21, 1181-1188.
(7) AlFakeeh, A.; Brezden-Masley, C. Overcoming endocrine resistance in hormone receptor-positive breast cancer. Curr. Oncol. 2018,25, S18-S27.
(8) Martin, L. A.; Ribas, R.; Simigdala, N.; Schuster, E.; Pancholi, S.; Tenev, T.; Gellert, P.; Buluwela, L.; Harrod, A.; Thornhill, A.; Nikitorowicz-Buniak, J.; Bhamra, A.; Turgeon, M. O.; Poulogiannis, G.; Gao, Q.; Martins, V.; Hills, M.; Garcia-Murillas, I.; Fribbens, C.; Patani, N.; Li, Z.; Sikora, M. J.; Turner, N.; Zwart, W.; Oesterreich, S.; Carroll, J.; Ali, S.; Dowsett, M. Discovery of naturally occurring ESR1 mutations in breast cancer cell lines modelling endocrine resistance. Nat. Commun. 2017, 8, 1865.
(9) Nardone, A.; De Angelis, C.; Trivedi, M. V.; Osborne, C. K.; Schifif, R. The changing role of ER in endocrine resistance. Breast 2015,24, S60-S66.
(10) Robertson, J. F.; Harrison, M. Fulvestrant: pharmacokinetics and pharmacology. Br. J. Cancer 2004, 90, S7-S10.
(11) Osborne, C. K.; Wakeling, A.; Nicholson, R. I. Fulvestrant: an oestrogen receptor antagonist with a novel mechanism of action. Br. J. Cancer 2004, 90, S2-S6.
(12) Howell, A.; Sapunar, F. Fulvestrant revisited: efficacy and safety of the 500-mg dose. Clin. Breast Cancer 2011, II, 204-210.
(13) Robertson, J. F.; Lindemann, J.; Garnett, S.; Anderson, E.; Nicholson, R. I.; Kuter, I.; Gee, J. M. A good chug made better: the fulvestrant dose-response story. Clin. Breast Cancer 2014, 14, 381-389.
(14) McDonnell, D. P.; Wardell, S. E.; Norris, J. D. Oral selective estrogen receptor downregulators (SERDs), a breakthrough endocrine therapy for breast cancer. J. Med Chem. 2015, 58, 4883-4887.
(15) Abdel-Magid, A. F. Selective estrogen receptor degraders (SERDs): a promising treatment to overcome resistance to endocrine therapy in ERα-positive breast cancer. ACS Med Chem. Lett. 2017, 8, 1129-1131.
(16) Weir, H. M.; Bradbury, R. H.; Lawson, M.; Rabow, A. A.; Buttar, D.; Callis, R. J.; Curwen, J. O.; de Almeida, C.; Ballard, P.; Hulse, M.; Donald, C. S.; Feron, L. J.; Karoutchi, G.; MacFaul, P.; Moss, T.; Norman, R. A.; Pearson, S. E.; Tonge, M.; Davies, G.; Walker, G. E.; Wilson, Z.; Rowlinson, R.; Powell, S.; Sadler, C.; Richmond, G.; Ladd, B.; Pazolli, E.; Mazzola, A. M.; D'Cruz, C.; De Savi, C. AZD9496: an oral estrogen receptor inhibitor that blocks the growth of ER-positive and ESR1-mutant breast tumors in preclinical models. Cancer Res. 2016, 76, 3307-3318.
(17) Joseph, J. D.; Darimont, B.; Zhou, W.; Arrazate, A.; Young, A.; Ingalla, E.; Walter, K.; Blake, R. A.; Nonomiya, J.; Guan, Z.; Kategaya, L.; Govek, S. P.; Lai, A. G.; Kahraman, M.; Brigham, D.; Sensintafifar, J.; Lu, N.; Shao, G.; Qian, J.; Grillot, K.; Moon, M.; Prudente, R.; Bischoff, E.; Lee, K. J.; Bonnefous, C.; Douglas, K. L.; Julien, J. D.; Nagasawa, J. Y.; Aparicio, A.; Kaufman, J.; Haley, B.; Giltnane, J. M.; Wertz, I. E.; Lackner, M. R.; Nannini, M. A.; Sampath, D.; Schwarz, L.; Manning, H. C.; Tantawy, M. N.; Arteaga, C. L.; Heyman, R. A.; Rix, P. J.; Friedman, L.; Smith, N. D.; Metcalfe, C.; Hager, J. H. The selective estrogen receptor downregulator GDC-0810 is efficacious in diverse models of ER+ breast cancer. eLife 2016, 5, e15828.
(18) Bihani, T.; Patel, H. K.; Arlt, H.; Tao, N.; Jiang, H.; Brown, J. L.; Purandare, D. M.; Hattersley, G.; Gamer, F. Elacestrant (RAD1901), a selective estrogen receptor degrader (SERD), has antitumor activity in multiple ER+ breast cancer patient-derived xenograft models. Clin. Cancer Res. 2017, 23, 4793-4804.
(19) Tria, G. S.; Abrams, T.; Baird, J.; Burks, H. E.; Firestone, B.; Gaither, L. A.; Hamann, L. G.; He, G.; Kirby, C. A.; Kim, S.; Lombardo, F.; Macchi, K. J.; McDonnell, D. P.; Mishina, Y.; Norris, J. D.; Nunez, J.; Springer, C.; Sun, Y.; Thomsen, N. M.; Wang, C.; Wang, J.; Yu, B.; Tiong-Yip, C. L.; Peukert, S. Discovery of LSZ102, a potent, orally bioavailable selective estrogen receptor degrader (SERD) for the treatment of estrogen receptor positive breast cancer. J. Med. Chem. 2018, 61, 2837-2864.
(20) Carlson, R. W. The history and mechanism of action of fulvestrant. Clin. Breast Cancer 2005, 6, S5-S8.
(21) Marsaud, V.; Gougelet, A.; Maillard, S.; Renoir, J. M. Various phosphorylation pathways, depending on agonist and antagonist binding to endogenous estrogen receptor a (ERα), differentially affect ERα extractability, proteasome-mediated stability, and transcriptional activity in human breast cancer cells. Mol. Endocrinol. 2003, 77, 2013-2027.
(22) Wittmann, B. M.; Sherk, A.; McDonnell, D. P. Definition of functionally important mechanistic differences among selective estrogen receptor down-regulators. Cancer Res. 2007, 67, 9549-9560.
(23) Sakamoto, K. M.; Kim, K. B.; Kumagai, A.; Mercurio, F.; Crews, C. M.; Deshaies, R. J. Protacs: chimeric molecules that target proteins to the Skpl-Cullin-F box complex for ubiquitination and degradation. Proc. Natl. Acad. Sci. U.S.A 2001, 98, 8554-8559.
(24) Toure, M.; Crews, C. M. Small-molecule PROTACS: new approaches to protein degradation. Angew. Chem., Int. Ed. 2016, 55, 1966-1973.
(25) Lai, A. C.; Crews, C. M. Induced protein degradation: an emerging chug discovery paradigm. Nat. Rev. Drug Discovery 2017, 16, 101-114.
(26) Burslem, G. M.; Crews, C. M. Small-molecule modulation of protein homeostasis. Chem. Rev. 2017, 117, 11269-11301.
(27) Cromm, P. M.; Crews, C. M. Targeted protein degradation: from chemical biology to drug discovery. Cell Chem. Biol. 2017, 24, 1181-1190.
(28) Ottis, P.; Crews, C. M. Proteolysis-targeting chimeras: induced protein degradation as a therapeutic strategy. ACS Chem. Biol. 2017, 12, 892-898.
(29) Rodriguez-Gonzalez, A.; Cyrus, K.; Salcius, M.; Kim, K.; Crews, C. M.; Deshaies, R. J.; Sakamoto, K. M. Targeting steroid hormone receptors for ubiquitination and degradation in breast and prostate cancer. Oncogene 2008, 27, 7201-7211.
(30) Jiang, Y.; Deng, Q.; Zhao, H.; Xie, M.; Chen, L.; Yin, F.; Qin, X.; Zheng, W.; Zhao, Y.; Li, Z. Development of stabilized peptide-based PROTACs against estrogen receptor a. ACS Chem. Biol. 2018, 13, 628-635.
(31) Winter, G. E. B., D. L.; Paulk, J.; Roberts, J. M.; Souza, A.; Dhe-Paganon, S. B., J. E. Phthalimide conjugation as a strategy for in vivo target protein degradation. Science 2015, 348, 1376-1381.
(32) Lu, J.; Qian, Y.; Altieri, M.; Dong, H.; Wang, J.; Raina, K.; Hines, J.; Winkler, J. D.; Crew, A. P.; Coleman, K.; Crews, C. M. Hijacking the E3 ubiquitin ligase cereblon to efficiently target BRD4. Chem. Biol. 2015, 22, 755-763.
(33) Bondeson, D. P.; Mares, A.; Smith, I. E.; Ko, E.; Campos, S.; Miah, A. H.; Mulholland, K. E.; Routly, N.; Buckley, D. L.; Gustafson, J. L.; Zinn, N.; Grandi, P.; Shimamura, S.; Bergamini, G.; Faelth-Savitski, M.; Bantscheff, M.; Cox, C.; Gordon, D. A.; Willard, R. R.; Flanagan, J. J.; Casillas, L. N.; Votta, B. J.; den Besten, W.; Famm, K.; Kruidenier, L.; Carter, P. S.; Harling, J. D.; Churcher, I.; Crews, C. M. Catalytic in vivo protein knockdown by small-molecule PROTACs. Nat. Chem. Biol. 2015, II, 611-617.
(34) Lai, A. C.; Toure, M.; Hellerschmied, D.; Salami, J.; Jaime-Figueroa, S.; Ko, E.; Hines, J.; Crews, C. M. Modular PROTAC design for the degradation of oncogenic BCR-ABL. Angew. Chem., Int. Ed 2016, 55, 807-810.
(35) Robb, C. M.; Contreras, J. I.; Kour, S.; Taylor, M. A.; Abid, M.; Sonawane, Y. A.; Zahid, M.; Murry, D. J.; Natarajan, A.; Rana, S. Chemically induced degradation of CDK9 by a proteolysis targeting chimera (PROTAC). Chem. Commun. 2017, 53, 7577-7580.
(36) Zhang, C.; Han, X. R.; Yang, X.; Jiang, B.; Liu, J.; Xiong, Y.; Jin, J. Proteolysis targeting chimeras (PROTACs) of anaplastic lymphoma kinase (ALK). Eur. J. Med Chem. 2018, 151, 304-314.
(37) Lu, M.; Liu, T.; Jiao, Q.; Ji, J.; Tao, M.; Liu, Y.; You, Q.; Jiang, Z. Discovery of a Keapl-dependent peptide PROTAC to knockdown Tau by ubiquitination-proteasome degradation pathway. Eur. J. Med Chem. 2018, 146, 251-259.
(38) Burslem, G. M.; Smith, B. E.; Lai, A. C.; Jaime-Figueroa, S.; McQuaid, D. C.; Bondeson, D. P.; Toure, M.; Dong, H.; Qian, Y.; Wang, J.; Crew, A. P.; Hines, J.; Crews, C. M. The advantages of targeted protein degradation over inhibition: an RTK case study. Cell Chem. Biol. 2018, 25, 67-77.
(39) Schiedel, M.; Herp, D.; Hammelmann, S.; Swyter, S.; Lehotzky, A.; Robaa, D.; Olah, J.; Ovadi, J.; Sippl, W.; Jung, M. Chemically induced degradation of sirtuin 2 (sirt2) by a proteolysis targeting chimera (PROTAC) based on sirtuin rearranging ligands (SirReals). J. Med Chem. 2018, 61, 482-491.
(40) Shibata, N.; Nagai, K.; Morita, Y.; Ujikawa, O.; Ohoka, N.; Hattori, T.; Koyama, R.; Sano, O.; Imaeda, Y.; Nara, H.; Cho, N.; Naito, M. Development of protein degradation inducers of androgen receptor by conjugation of androgen receptor ligands and inhibitor of apoptosis protein ligands. J. Med Chem. 2018, 61, 543-575.
(41) Crew, A. P.; Raina, K.; Dong, H.; Qian, Y.; Wang, J.; Vigil, D.; Serebrenik, Y. V.; Hamman, B. D.; Morgan, A.; Ferraro, C.; Siu, K.; Neklesa, T. K.; Winkler, J. D.; Coleman, K. G.; Crews, C. M. Identification and characterization of von Hippel-Lindau-recruiting proteolysis targeting chimeras (PROTACs) of TANK-binding kinase 1. J Med Chem. 2018, 61, 583-598.
(42) Sun, Y.; Zhao, X.; Ding, N.; Gao, H.; Wu, Y.; Yang, Y.; Zhao, M.; Hwang, J.; Song, Y.; Liu, W.; Rao, Y. PROTAC-induced BTK degradation as a novel therapy for mutated BTK C481S induced ibrutinib-resistant B-cell malignancies. Cell Res. 2018, 28, 779-781.
(43) Yang, K.; Song, Y.; Xie, H.; Wu, H.; Wu, Y. T.; Leisten, E. D.; Tang, W. Development of the first small molecule histone deacetylase 6 (HDAC6) degraders. Bioorg. Med Chem. Lett. 2018, 28, 2493-2497.
(44) Ohoka, N.; Okuhira, K.; Ito, M.; Nagai, K.; Shibata, N.; Hattori, T.; Ujikawa, O.; Shimokawa, K.; Sano, O.; Koyama, R.; Fujita, H.; Teratani, M.; Matsumoto, H.; Imaeda, Y.; Nara, H.; Cho, N.; Naito, M. In vivo knockdown of pathogenic proteins via specific and nongenetic inhibitor of apoptosis protein (IAP)-dependent protein erasers (SNIPERs). J. Biol. Chem. 2017, 292, 4556-4570.
(45) Ohoka, N.; Morita, Y.; Nagai, K.; Shimokawa, K.; Ujikawa, O.; Fujimori, I.; Ito, M.; Hayase, Y.; Okuhira, K.; Shibata, N.; Hattori, T.; Sameshima, T.; Sano, O.; Koyama, R.; Imaeda, Y.; Nara, H.; Cho, N.; Naito, M. Derivatization of inhibitor of apoptosis protein (IAP) ligands yields improved inducers of estrogen receptor a degradation. J Biol. Chem. 2018, 293, 6776-6790.
(46) Ottis, P.; Toure, M.; Cromm, P. M.; Ko, E.; Gustafson, J. L.; Crews, C. M. Assessing different E3 ligases for small molecule induced protein ubiquitination and degradation. ACS Chem. Biol. 2017, 12, 2570-2578.
(47) Kanak Rainaa, J. L., Yimin Qiana, Martha Altieria, Deborah Gordona, Ann Marie K. Rossia, Jing Wanga, Xin Chena, Hanqing Donga, Kam Siua, James D. Winklera, Andrew P. Crewa, Craig M. Crews and Kevin G. Colemana. PROTAC-induced BET protein degradation as a therapy for castration-resistant prostate cancer. Proc. Natl. Acad Sci. U.S.A 2016, 113, 7124-7129.
(48) Zhou, B.; Hu, J.; Xu, F.; Chen, Z.; Bai, L.; Femandez-Salas, E.; Lin, M.; Liu, L.; Yang, C. Y.; Zhao, Y.; McEachem, D.; Przybranowski, S.; Wen, B.; Sun, D.; Wang, S. Discovery of a small-molecule degrader of bromodomain and extra-terminal (BET) proteins with picomolar cellular potencies and capable of achieving tumor regression. J. Med Chem. 2018, 61, 462-481.
(49) Andrzej M. Brzozowski, A. C. W. P., Zbigniew Dauter, Roderick E. Hubbard, Tomas Bonn, Owe Engstro, Lars O″ hman, Geoffrey L. Greene, Jan-A° ke Gustafsson, Mats Carlquist. Molecular basis of agonism and antagonism in the oestrogen receptor. Nature 1997, 389, 753-758.
(50) Qin, C.; Hu, Y.; Zhou, B.; Femandez-Salas, E.; Yang, C. Y.; Liu, L.; McEachem, D.; Przybranowski, S.; Wang, M.; Stuckey, J.; Meagher, J.; Bai, L.; Chen, Z.; Lin, M.; Yang, J.; Ziazadeh, D. N.; Xu, F.; Hu, J.; Xiang, W.; Huang, L.; Li, S.; Wen, B.; Sun, D.; Wang, S. Discovery of QCA570 as an exceptionally potent and efficacious proteolysis targeting chimera (PROTAC) degrader of the bromodomain and extra-terminal (BET) proteins capable of inducing complete and durable tumor regression. J. Med Chem. 2018, 61, 6685-6704.
(51) Bai, L.; Zhou, B.; Yang, C. Y.; Ji, J.; McEachem, D.; Przybranowski, S.; Jiang, H.; Hu, J.; Xu, F.; Zhao, Y.; Liu, L.; Femandez-Salas, E.; Xu, J.; Dou, Y.; Wen, B.; Sun, D.; Meagher, J.; Stuckey, J.; Hayes, D. F.; Li, S.; Ellis, M. J.; Wang, S. Targeted degradation of BET proteins in triple-negative breast cancer. Cancer Res. 2017, 77, 2476-2487.
(52) Buckley, D. L.; Van Molle, I.; Gareiss, P. C.; Tae, H. S.; Michel, J.; Noblin, D. J.; Jorgensen, W. L.; Ciulli, A.; Crews, C. M. Targeting the von Hippel-Lindau E3 ubiquitin ligase using small molecules to disrupt the VHL/HIF-1α interaction. J Am. Chem. Soc. 2012, 134, 4465-4468.
(53) Buckley, D. L.; Gustafson, J. L.; Van Molle, I.; Roth, A. G.; Tae, H. S.; Gareiss, P. C.; Jorgensen, W. L.; Ciulli, A.; Crews, C. M. Small-molecule inhibitors of the interaction between the E3 ligase VHL and HIF-1α. Angew. Chem., Int. Ed 2012, 51, 11463-11467.
(54) Galdeano, C.; Gadd, M. S.; Soares, P.; Scaffidi, S.; Van Molle, I.; Birced, I.; Hewitt, S.; Dias, D. M.; Ciulli, A. Structure-guided design and optimization of small molecules targeting the protein-protein interaction between the von Hippel-Lindau (VHL) E3 ubiquitin ligase and the hypoxia inducible factor (HIF) a subunit with in vitro nanomolar affinities. J. Med Chem. 2014, 57, 8657-8663.
(55) Soares, P.; Gadd, M. S.; Frost, J.; Galdeano, C.; Ellis, L.; Epemolu, O.; Rocha, S.; Read, K. D.; Ciulli, A. Group-based optimization of potent and cell-active inhibitors of the von Hippel-Lindau (VHL) E3 ubiquitin ligase: structure-activity relationships leading to the chemical probe (2S,4R)-1-((S)-2-(1-cyanocyclopropanecarboxamido)-3,3-dimethylbutanoyl)-4-hydroxy-N-(4-(4-methylthiazol-5-yl)benzyl)pyrrolidine-2-carboxamide (VH298). J. Med Chem. 2018, 61, 599-618.
(56) Gadd, M. S.; Testa, A.; Lucas, X.; Chan, K. H.; Chen, W.; Lamont, D. J.; Zengerle, M.; Ciulli, A. Structural basis of PROTAC cooperative recognition for selective protein degradation. Nat. Chem. Biol. 2017, 13, 514-521.
(57) Long, M. J.; Poganik, J. R.; Aye, Y. On-demand targeting: investigating biology with proximity-directed chemistry. J. Am. Chem. Soc. 2016, I38, 3610-3622.
(58) Stols, L.; Gu, M.; Dieckman, L.; Rafifen, R.; Collart, F. R.; Donnelly, M. I. A new vector for high-throughput, ligation-independent cloning encoding a tobacco etch virus protease cleavage site. Protein Expr. Purif. 2002, 25, 8-15.
(59) Benoit, R. M.; Ostermeier, C.; Geiser, M.; Li, J. S.; Widmer, H.; Auer, M. Seamless insert-plasmid assembly at high efficiency and low cost. PLoS One 2016, II, e0153158.

It is to be understood that the foregoing embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I:
A-L-B I,
wherein:
A is a radical of an estrogen receptor modulator selected from the group consisting of: 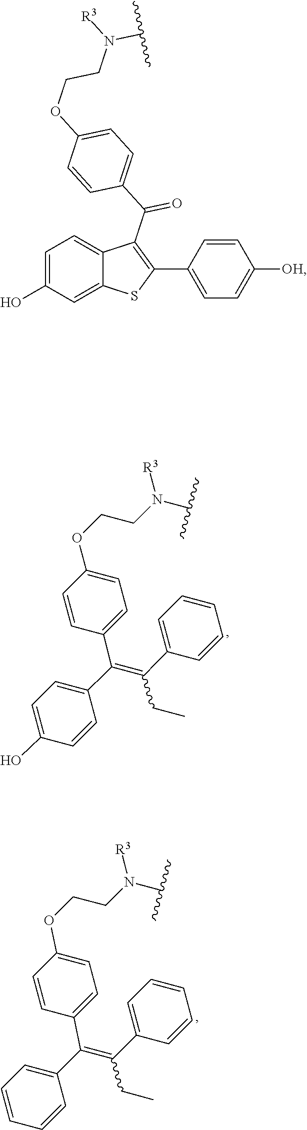 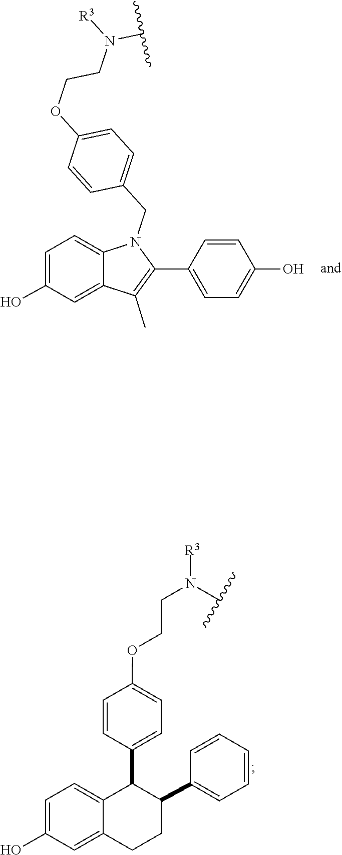
R³ is selected from the group consisting of C₁-C₆ alkyl, C₃-C₈ cycloalkyl, and (C₃-C₈ cycloalkyl)C₁-C₄ alkyl;
L is a linker; and
B is a radical of an E3 ligase ligand selected from the group consisting of: 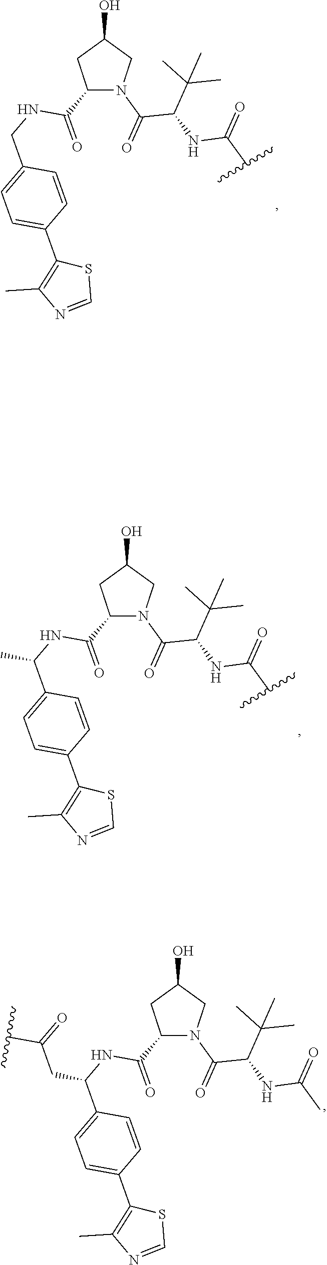 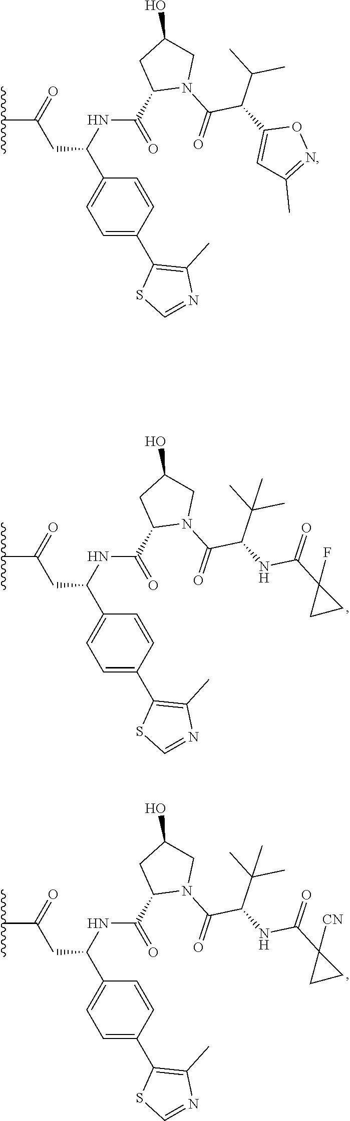 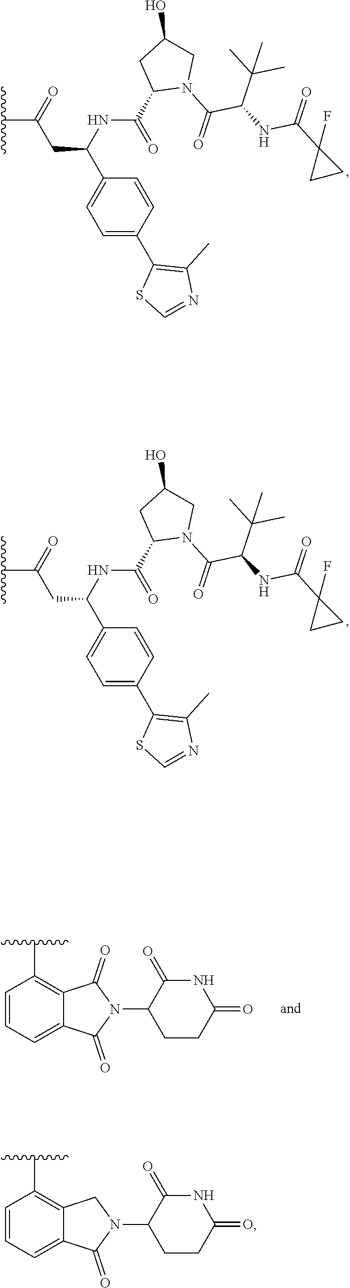
or a pharmaceutically acceptable salt or solvate thereof.

2. The compound of claim 1, wherein A is selected from the group consisting of: 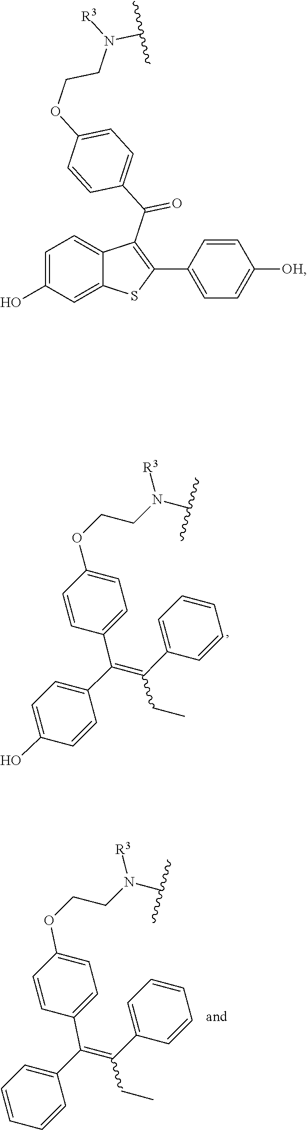 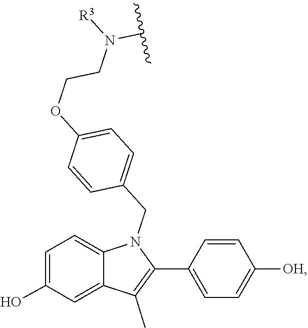
or a pharmaceutically acceptable salt or solvate thereof.

3. The compound of claim 2, wherein B is selected from the group consisting of: 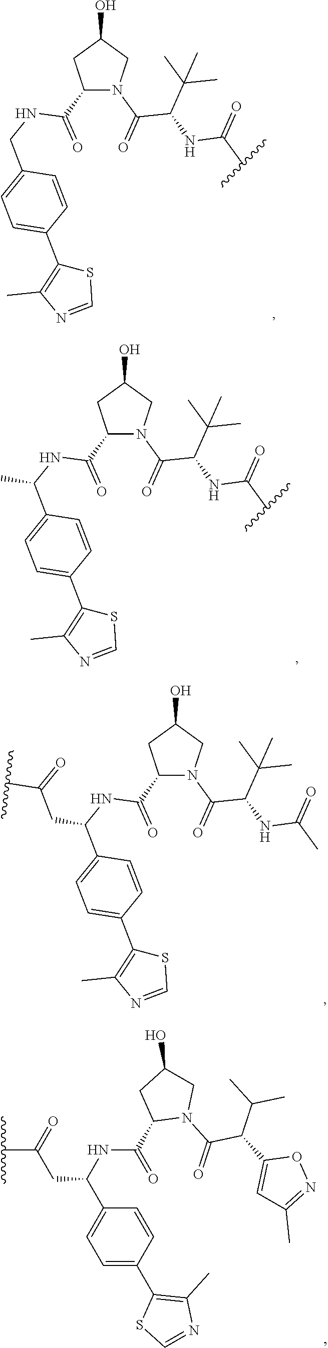 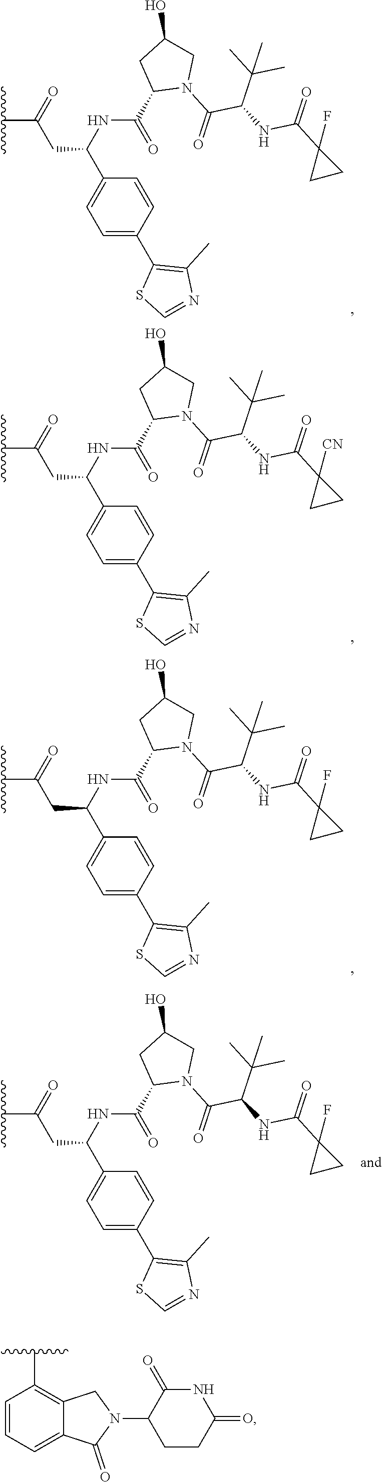
or a pharmaceutically acceptable salt or solvate thereof.

4. The compound of claim 1 having Formula III: 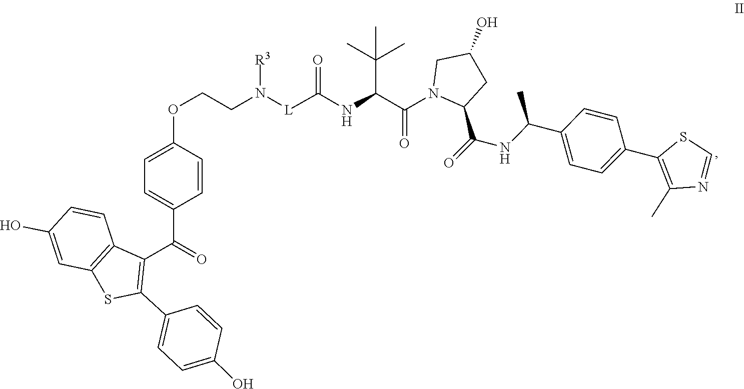
or a pharmaceutically acceptable salt or solvate thereof.

5. The compound of claim 1 having Formula III: 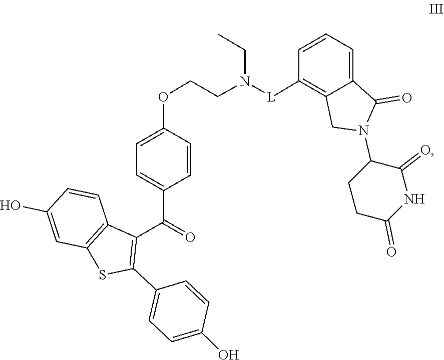
or a pharmaceutically acceptable salt or solvate thereof.

6. The compound of any one of claims 1-5, wherein
L is —X-L¹-Z—;
X is selected from the group consisting of —C≡C—, —O—, —C(═O)N(R^{1a})—, and —N(R^{3a})—; or
X is absent;
Z is selected from the group consisting of —C≡C—, —O—, —C(═O)N(R^{2a})—, and —N(R^{4a})—; or
Z is absent;
L¹ is selected from the group consisting of alkylenyl, heteroalkylenyl, and —W¹—(CH₂)ₘ—W²—(CH₂)ₙ—
W¹ is absent; or
W¹ is selected from the group consisting of phenylenyl, heteroarylenyl, heterocyclenyl, and cycloalkylenyl;
W² is selected from the group consisting of phenylenyl, heteroarylenyl, heterocyclenyl, and cycloalkylenyl;
m is 0, 1, 2, 3, 4, 5, 6, or 7;
n is 0, 1, 2, 3, 4, 5, 6, 7, or 8; and
R^{1a} is selected from the group consisting of hydrogen and C₁₋₄ alkyl;
R^{2a} is selected from the group consisting of hydrogen and C₁₋₄ alkyl;
R^{3a} is selected from the group consisting of hydrogen and C₁₋₄ alkyl; and
R^{4a} is selected from the group consisting of hydrogen and C₁₋₄ alkyl,
or a pharmaceutically acceptable salt or solvate thereof.

7. The compound of claim 9, wherein L is selected from the group consisting of: 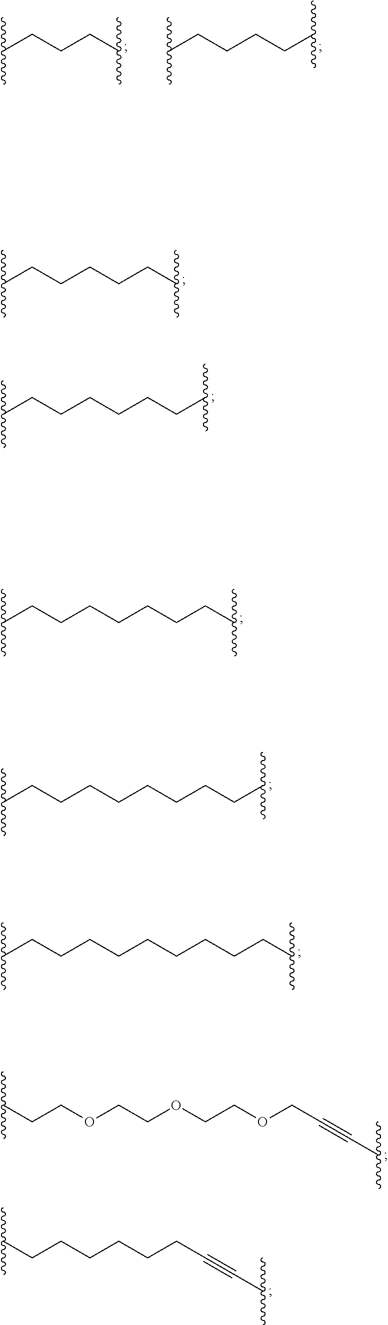 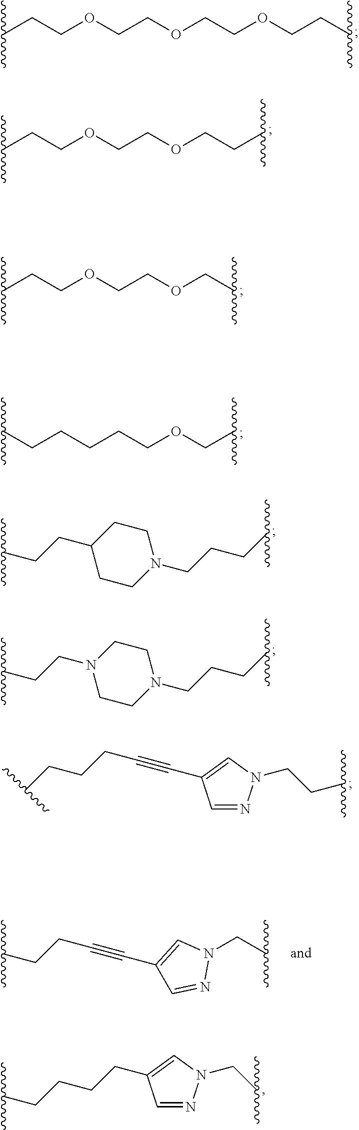
or a pharmaceutically acceptable salt or solvate thereof.

8. The compound of claim 1 or 2 having Formula IV: 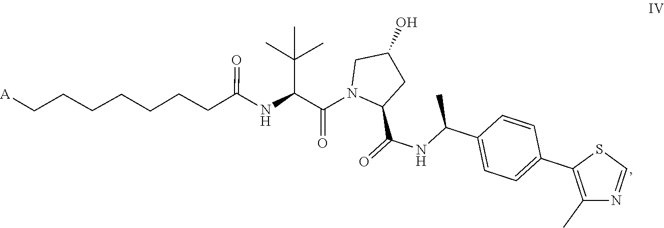
or a pharmaceutically acceptable salt or solvate thereof.

9. A compound having Formula V: 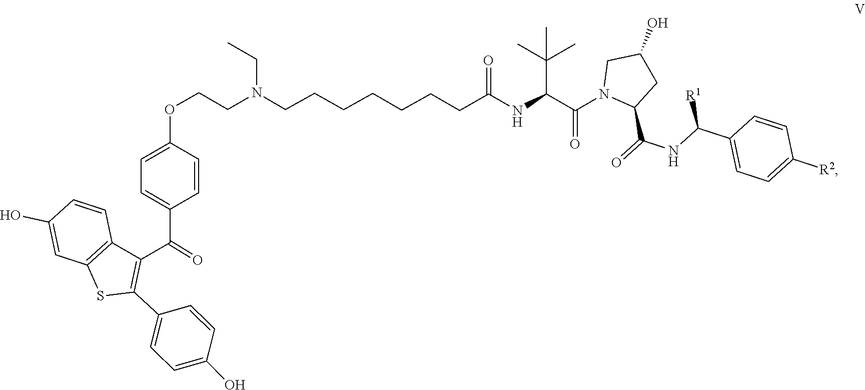
wherein:
R¹ is selected from the group consisting of hydrogen and C₁-C₃ alkyl; and
R² is selected from the group halo, cyano, C₂-C₄ alkynyl, C₁-C₆ alkyl, and C₃-C₆ cycloalkyl
or a pharmaceutically acceptable salt or solvate thereof.

10. A pharmaceutical composition comprising a compound of any one of claims 1-9, or a pharmaceutically acceptable salt or solvate thereof, and a pharmaceutically acceptable excipient.

11. A method of treating cancer in a patient in need thereof, the method comprising administering to the subject a pharmaceutically effective amount of a compound of any one of claims 1-12, or a pharmaceutically acceptable salt or solvate thereof.

12. The method of claim 11, wherein the cancer is breast cancer.

13. The method of claim 11 or 12, wherein the compound is administered in combination with a second anticancer agent.

14. The method of claim 13, wherein the second anticancer agent is selected from the group consisting of abemaciclib, paclitaxel, ado-trastuzumab emtansine, afinitor, anastrozole, pamidronate disodium, exemestane, capecitabine, docetaxel, doxorubicin hydrochloride, epirubicin hydrochloride, eribulin mesylate, exemestane, fluorouracil, toremifene, fulvestrant, letrozole, gemcitabine hydrochloride, goserelin acetate, trastuzumab, palbociclib, ixabepilone, ribociclib, lapatinib ditosylate, olaparib, megestrol acetate, methotrexate, neratinib maleate, palbociclib, pamidronate disodium, pertuzumab, tamoxifen citrate, taxotere, thiotepa, toremifene, trastuzumab, and vinblastine sulfate.
